(19) United States
(12) Patent Application Publication
(10) Pub. No.: US 2015/0079071 A1
(43) Pub. Date: Mar. 19, 2015

(54) NON-CATALYTIC DOMAIN TARGETS IN MATRIX METALLOPROTEASE PROTEINS FOR CANCER THERAPIES

(71) Applicants: Mina J. Bissell, Berkeley, CA (US); Celeste M. Nelson, Princeton, NJ (US); Hidetoshi Mori, Albany, CA (US); Ana Luisa Pinto Correia, Lamego (PT); Emily Chen, Stony Brook, NY (US)

(72) Inventors: Mina J. Bissell, Berkeley, CA (US); Celeste M. Nelson, Princeton, NJ (US); Hidetoshi Mori, Albany, CA (US); Ana Luisa Pinto Correia, Lamego (PT); Emily Chen, Stony Brook, NY (US)

(73) Assignee: THE REGENTS OF THE UNIVERSITY OF CALIFORNIA, Oakland, CA (US)

(21) Appl. No.: 14/094,702

(22) Filed: Dec. 2, 2013

Related U.S. Application Data
(60) Provisional application No. 61/732,231, filed on Nov. 30, 2012.
(60) Provisional application No. 61/862,196, filed on Aug. 5, 2013.

(51) Int. Cl. C07K 16/40; C12N 15/115; C12N 15/113

(52) U.S. Cl. CPC C07K 16/40; C12N 15/1137; C12N 15/115; C12N 2310/11; C12N 2310/16; C07K 2317/76; C07K 2317/24
USPC 424/133.1; 514/44 A; 424/139.1; 435/184

(57) ABSTRACT

The present invention provides compositions and methods for inhibiting MMPs, especially MMP-3, MMP-9 and MMP-14. The invention relates to the field of diagnostic and prognostic methods of human cancers, especially breast cancer and the field of matrix metalloproteases, as targets for diagnostics and therapeutics.

### CROSS-REFERENCE TO RELATED APPLICATIONS

This application is a non-provisional application of and claiming priority to U.S. Provisional Patent Application Nos. 61/732,231 filed on Nov. 30, 2012 and 61/862,196 filed on Aug. 5, 2013, both of which are hereby incorporated by reference in their entireties.

### STATEMENT OF GOVERNMENTAL SUPPORT

The invention was made with government support under Contract No. DE-AC02-05CH1123 awarded by the U.S. Department of Energy, Office of Biological and Environmental Research and under Contract No. W81XWH0810736 awarded by the U.S. Department of Defense, and under Grant Nos. CA064786, CA057621, CA140663, CA112970, CA143233, and CA143836 awarded by the National Cancer Institute of the National Institutes of Health. The government has certain rights in the invention.

### CROSS-REFERENCE TO ATTACHED SEQUENCE LISTING APPENDIX

The informal sequence listing attached as an appendix is hereby incorporated by reference in its entirety.

### BACKGROUND OF THE INVENTION

1. Field of the Invention

The invention relates to the field of diagnostic and prognostic methods of human cancers, especially breast cancer. The invention also relates to the field of matrix metalloproteases, as targets for diagnostics and therapeutics.

2. Related Art

Cancer is characterized by a progressive series of alterations that disrupt cell and tissue homeostasis. Whereas many of these alterations can be induced by specific mutations, faulty signals from the microenvironment also can act as inducers of tumor development and progression (Bissell, M. J. & Radisky, D. Putting tumours in context. Nat Rev Cancer 1, 46-54, 2001). Matrix metalloproteinases (MMPs) are prominent contributors to such microenvironmental signals. MMPs are proteolytic enzymes that degrade structural components of the extracellular matrix (ECM), allowing for tumor invasion and metastasis. Additionally, MMPs can release cell-bound inactive precursor forms of growth factors, degrade cell-cell and cell-ECM adhesion molecules, activate precursor zymogen forms of other MMPs, and inactivate inhibitors of MMPs and other proteases (Egeblad, M. & Werb, Z. New functions for the matrix metalloproteinases in cancer progression. Nat Rev Cancer 2, 161-74, 2002). MMPs have been shown to be a causative factor in number of cancers including, e.g., cancers of the lung, breast, colon, skin, prostate, ovary, pancreas, uroepithelial cells, squamous cells, tongue, mouth, and stomach.

Due to the role of MMP in tumorigenesis and metastasis, compositions and methods for treating and preventing cancer by specifically targeting MMP's have been explored. However, attempts to treat or prevent cancer by directly inhibiting MMP's have not been successful in the clinic. Cancer patients receiving MMP inhibitors experienced a number of deleterious side effects (e.g., inflammation and acute pain) that led to cessation of the clinical trials and/or administration of drastically reduced doses of the MMP inhibitors in subsequent phases of the clinical trials (Coussens et al., Science 295: 2387, 2002).

Additionally, the formation of branched organs involves coordinated invasion of epithelium into the surrounding stroma (Chuong, 1998; Fata et al., 2004; Lu and Werb, 2008; Yamada and Cukierman, 2007). In the mammary gland (MG), both ductal branching and alveologenesis require integrin mediated signaling as well as the activity of matrix metalloproteinases (MMPs) (Fata et al., 2007; Simian et al., 2001; Sympson et al., 1994; Talhouk et al., 1991). These and other studies have demonstrated roles for secreted MMPs in the developing MG (review in Fata et al., 2004). Whereas the primary source of Mmp2 and Mmp3 is the mammary stroma, Mmp14 was shown to be expressed in both mammary stroma and epithelium of terminal end buds (TEBs) (Wiseman et al., 2003), suggesting that MMP14 may be involved in epithelial invasion into the mammary fat pad. However, despite its position on the cell surface and its expression in TEBs, neither a role for MMP14 in mammary branching morphogenesis nor its possible mechanism of action have been explored.

Thus, there is a need in the art for compositions and methods for detecting expression of proteins that play a role in MMP-induced malignant transformation as well as methods and composition for modulating proteins that play a role in MMP-induced malignancy. The present invention satisfies these and other needs.

### BRIEF SUMMARY OF THE INVENTION

The present invention provides for a method for inhibiting matrix metalloproteinase (MMP) proteins comprising contacting a cell with a compound that binds to a non-catalytic MMP domain and inhibits a biological effect. In various embodiments, the matrix metalloproteinase (MMP) is MMP-3, MMP-9, or MMP-14.

In one embodiment, the matrix metalloproteinase (MMP) is MMP-14 and the non-catalytic MMP-14 domain comprises residues 318-582 or any fragments thereof. In some embodiments, the non-catalytic MMP-14 domain comprises residues 318-523 or any fragments thereof. In other embodiments, the non-catalytic MMP domain comprises residues 542-562

The compound may comprise a siRNA, antibody, antisense oligonucleotide or aptamer sequence or other inhibitory compound. In some embodiments, the compound comprises a siRNA that binds a non-catalytic MMP domain. In other embodiments, the compound comprises an antibody that specifically binds to a non-catalytic MMP domain. In specific embodiments, the antibody is a monoclonal antibody and in further embodiments, the antibody is humanized.

The compounds once administered bind to the non-catalytic domains of one of the MMP proteins and modulate or inhibit a biological effect. In some embodiments, the biologic effect is metastasis, cell migration, tumorgenesis, and/or tumor invasion.

In another embodiment, the matrix metalloproteinase (MMP) is MMP-3. In some embodiments, the non-catalytic MMP domain comprises residues 289-477 or any fragments thereof. The inhibited or bound non-catalytic MMP-3 domain may comprise any of the hemopexin domains comprising residues 287-336 Hemopexin 1; residues 337-383 Hemopexin 2; residues 385-433 Hemopexin 3; and residues 434-477 Hemopexin 4 and/or any fragments thereof.

A method for inhibiting cancer progression comprising contacting a cell with a compound that binds to a non-catalytic MMP-14 domain and inhibits metastasis, cell migration, tumorgenesis, or tumor invasion. In some embodiments, the non-catalytic MMP14 domain that is bound or inhibited is the transmembrane/cytoplasmic domain.

In various embodiments, the compound comprises a siRNA, antibody, antisense oligonucleotide or aptamer sequence. In some embodiments, the compound comprises a siRNA that binds a MMP14 non-catalytic domain. In another embodiment, the compound comprises an antibody that specifically binds to a MMP14 domain and/or inhibits a MMP14 domain. In other embodiments, the antibody is a monoclonal antibody and in some embodiments is humanized.

In various embodiments, the invention further provides for antibodies to any non-catalytic domain of MMP-3, MMP-9 or MMP-14. In some embodiments, antibodies to non-catalytic MMP-3 hemopexin domains comprising residues 287-336 Hemopexin 1; residues 337-383 Hemopexin 2; residues 385-433 Hemopexin 3; and residues 434-477 Hemopexin 4 and/or any fragments thereof; or antibodies to any non-catalytic MMP-14 domain comprising residues 318-582, residues 318-523, or residues 542-562 or any fragments thereof.

### BRIEF DESCRIPTION OF THE FIGURES

FIG. 1. Mmp14 expression peaks during puberty, and is highly elevated at the invading front of mammary gland (MG) end buds. (a) Quantitative RT-PCR analysis of Mmp14 expression during development of the mouse MG from: virgin (V: 2-3, 6-8, and 9-14 weeks after birth), early pregnancy (Early-preg; day 4), mid-pregnancy (Mid-preg; days 8-12), late pregnancy (Late-Preg; days 16-18) and lactating (days 1-10), normalized to 18S rRNA. Data are mean+/−S.E.; (**) indicate p<0.01 when compared to V (2-3w). (b) Mmp14 promoter activity in MGs from Mmp14 (+/lacZ) mice. Data are from glands of 5 week old mice. β-gal staining of a whole mount of a MG isolated from a virgin transgenic heterozygote mouse bearing the lacZ gene under the control of the endogenous Mmp14 promoter (Yana et al., 2007) indicates that Mmp14 promoter activity is high in mammary epithelial cells. (i) β-gal stained MG from Mmp14 (+/+) mouse as negative control. (ii) β-gal stained MG from Mmp14 (+/lacZ) mouse. Scale bar: 6.25 mm. (iii) β-gal-stained mammary end buds in Mmp14 (+/lacZ) mouse showed intense promoter activity at the tip of the bud. (iv) β-gal and eosin stain of a MG tissue section from a 5 week old Mmp14 (+/lac Z) transgenic mouse. Scale bars: 200 μm.

FIG. 2. Mmp14 catalytic activity is required for invasion/branching only in dense—but not sparse—collagen gels. (a) Schematic of a 3D organotypic culture model of mammary epithelial cell invasion/branching. Mammary organoids from Balb/c mice or clusters of EpH4 cells were induced to branch by addition of 9 nM FGF2 for 5 days in either 3 mg/ml or 1 mg/ml collagen 1 (CL-1) (b) CL-1 (3 mg/ml): (i, iii) Vehicle control (ctrl; DMSO), (ii, iv) MMP inhibitor GM6001 (40 μM), or pre-treated with: (v) Control shRNA, or (vi) Mmp14-shRNA. (i, ii) Bright field image of primary organoids in 3 mg/ml CL-1 gel. (iii-vi) Live dye-(Calcein AM)-stained EpH4 cell aggregates in 3 mg/ml CL-1 gel. (vii) Invasion/branching of EpH4 cells was scored as positive when displaying three or more branches of a length at least half the diameter of the central cell cluster as described in Material and Methods. Percentages of cell invasion of ctrl (DMSO-treated; red bar) EpH4 cells vs. EpH4 treated with GM6001 or infected with control (ctrl_shRNA; green bar) and shMmp14-containing lentivirus (Mmp14_shRNA). 200 colonies were analyzed for each condition in 3 separate experiments. Data are mean+/−S.E.; (***) indicates p<0.001 compared to ctrl. (c) Invasion/branching of MECs in sparse CL-1 gels (1 mg/ml) in the presence of (i, iii) vehicle control (ctrl; DMSO), (ii, iv) MMP inhibitor GM6001 (40 □M), or pre-treated with (v) control-, or (vi) Mmp14-shRNA. (i, ii) bright field image of primary organoids in CL-1 gel. (iii-vi) Live dye (Calcein AM) stained EpH4 cell aggregates in CL-1 gel. (vii) Percentages of cells invading: EpH4 cells ctrl (DMSO-treated; red bar),) EpH4 treated with GM6001 (blue bar); EpH4 cells infected with control shRNA (ctrl, green bar)- or EpH4 cells infected with Mmp14 shRNA-containing lentivirus. 200 colonies were analyzed for each condition over 3 separate experiments. Data are mean+/−S.E.; (***) indicates p<0.001 when compared to ctrl. Scale bars: 200 μm.

FIG. 3. MAPK activity and cross signaling between Mmp14, Erk and Itgb1 are involved in branching morphogenesis. (a) Silencing Mmp14 reduces Erk activity and the level of Itgb1. (i) Immunoblots of phospho-Erk (pErk) in ctrl- and Mmp14-silenced EpH4 cells. (ii) Quantification of Erk activity in (i). Data are mean+/−S.E.; (*) indicates p<0.05. n=3. (iii) Western blot of Mmp14 and Itgb1 in ctrl- and Mmp14-silenced EpH4 cells. LAM A/C was used as the loading control. (iv) MECs from Mmp14 knockout had reduced level of Itgb1. Immunofluorescence intensity of Itgb1 measured in MG tissues from Mmp14 (+/−, HET) or Mmp14 (−/−, KO) mice (also see Fig. S5). Analysis was performed on luminal epithelial cells (LEP) and myoepithelial cells (MEP). Measurement was performed with IMARIS software (Bitplane). ≧50 cells were analyzed per tissue section, n=3 tissue sections. (****) indicates P<0.0001. (b) Silencing Itgb1 reduced MEC branching, MAPK activity and the Mmp14 levels in sparse CL-1 gels. Branching of (i) Control- or (ii) Itgb1-shRNA treated MECs in CL-1 gels of 1 mg/ml. Scale bar: 200 μm. (iii) Silencing Itgb1 reduced Erk phosphorylation. (iv) Quantification of the ratio between pErk and total Erk in Itgb1-shRNA treated EpH4 cells in sparse CL-1. Data are mean+/−S.E., with (*) indicating p<0.05. (v) Silencing Itgb1 reduced the expression levels of Mmp14 (mean intensity values normalized to Lamin A/C (LAM A/C) calculated via band densitometry from n=3 immunoblots shown below each band). (c) MEK activity is required for cell invasion in CL-1. (i, ii) Control (ctrl; DMSO) and PD98059-treated EpH4 cells in CL-1 gels of 1 mg/ml. (iii) MEK inhibition reduced Mmp14 and Itgb1 levels as determined by immunoblot (normalized mean intensity values calculated as above are given below each band). Scale bar: 200 □m.

FIG. 4. Co-immunoprecipitation and FRET reveal a direct association between Mmp14 and Itgb1. (a) Co-immunoprecipitation of endogenous Mmp14 and Itgb1. Protein complexes containing Mmp14 were immunoprecipitated from EpH4 cells cultured on collagen-1 coated dish and probed for Mmp14 (top row) or Itgb1 (bottom row). (b) FRET analysis of monomeric Cypet-tagged MMP14F (FLAG tagged human MMP14) and monomeric Ypet-tagged ITGB1 exogenously expressed in EpH4 MECs. Ypet emission signal was detected as FRET signal when Cypet was excited.

FIG. 5. Assigning functional activity to the non-catalytic domains of MMP14. (a) Scheme of FLAG tagged full length human MMP14 (MMP14F FL), catalytic domain deleted mutant (MMP14F dCAT) and catalytic/hemopexin domain deleted mutant (MMP14F dCAT/dPEX). (b) MMP14 overexpression rescued the level of Itgb1 in Mmp14 silenced cells. Expression of MMP14F-FL or the mutants was performed on Mmp14 silenced EpH4 cells at the passage 3 after infection with Mmp14 shRNA containing lenti virus. Samples for cell lysate or branching were used at passage 3 or 4 from silencing Mmp14. Immunoblot analysis of MMP14 (with an anti-FLAG antibody), Itgb1 (total and phospho-T788/T789) are indicated. The level of total and phospho-T788/T789 were up modulated when cells overexpressed MMP14F-FL or MMP14F-dCAT/dPEX. LAM A/C is shown as loading control. Numbers below blots indicate the ratio between Itgb1 (or phospho-T788/T789) and LAM A/C. (c) Full-length MMP14 or MMP14F dCAT/dPEX (i.e., only the transmembrane/cytoplasmic domain) mutant rescued invasion/branching in Mmp14-silenced EpH4 cells in sparse CL-1 gels. (i-iv) Mmp14-silenced EpH4 cells were infected with lentivirus containing (i) control lentivirus (mYpet), mYpet tagged- (ii) MMP14F-FL, (iii) MMP14F-dCAT and (iv) MMP14F-dCAT/dPEX, respectively. Cells were cultured in sparse (1 mg/ml) CL-1 gel. Scale bar: 40 □m.

FIG. 6. Schematic presentation of different steps involving Mmp14 and Itgb1 during MEC invasion/branching in a collagen-1 microenvironment. (a) Non-proteolytic activity of MMP14 is involved in mammary epithelial cell sorting in a CL-1 microenvironment (Mori et al., 2009). Schematic presentation of the relationship between Mmp14 expression and MEC sorting. Whereas MECs expressing full-length Mmp14 (FL-Mmp14) or the catalytic domain-deleted mutant (dCAT) sort to the invasive front, the hemopexin domain deleted mutant (dPEX) or MECs with silenced Mmp14 expression do not. (b) Proteolytic activity of Mmp14 is required for MECs to invade/branch in dense CL-1 (Alcaraz et al., 2011). MECs need to degrade collagen-1 to generate a path for invasion/branching in dense collagen. Mmp14 is at the hub of this proteolytic activity for collagen degradation. (c) Schematics of the association between Mmp14 and Itgb1 during MEC invasion/branching in sparse CL-1 microenvironment. Whereas MECs do not need MMP activity for invasion/branching in a sparse CL-1 gels, Mmp14 itself is required. Specifically, Mmp14 association with Itgb1 is necessary for MEC invasion/branching. Expressing FL-MMP14 or dCAT/dPEX in Mmp14-silenced MECs results in restoration of Itgb1 levels and activity to facilitate branching. Expression of the catalytic domain deleted mutant (dCAT) was unable to rescue branching and the activation of Itgb1 in a sparse CL-1 gels when the catalytic domain is absent. These events (from a to c) suggest that cells utilize different functions and domains of Mmp14 in a context-dependent manner during branching in collagenous microenvironments.

FIG. 7. Mammary epithelial cells in vivo are surrounded by type-I collagen. The MG from virgin C57BL/6 mouse at 8 weeks was cryosectioned and stained for type I collagen (upper and lower panels; green), alpha-smooth muscle actin (upper panels; red) and laminin alpha1 (lower panels; red). Type I collagen is present in basement membrane which surrounds the mammary-ducts. Anti-type I collagen (Chemicon), anti-alpha-smooth muscle actin (Sigma) and anti-laminin alpha1 (kind gift from Dr. Srorokin, University of Munster) were used as primary antibodies, and fluorescence conjugated goat anti-mouse IgG or anti-rabbit IgG (Invitrogen) were used for visualizing images. All images were captured by confocal microscopy (Solamere Technology Group). Scale bar: 100 μm.

FIG. 8. Quantitative RT-PCR analysis of Mmp14-silenced EpH4 cells. Quantitative RT-PCR was performed to confirm a loss Mmp14 expression in Mmp14 silenced EpH4 cells. n=3. Data are mean+/−

FIG. 9. Atomic Force Microscopy measurements confirmed the physiological collagen density and stiffness in 3D CL-1 gels. Atomic force microscopy (AFM) analysis of dense (3 mg/ml) and sparse (1 mg/ml) CL-1 gel stiffness for attached and floating CL-1 gels. Data are mean+/−S.E., with (***) indicating p<0.001.

FIG. 10. Metalloproteinase activity is not required for Erk1/2 phosphorylation in collagen-1 gels. EpH4 cells were cultured in collagen for 24 hours with or without GM6001 (40 □M). Ratio between pErk and total Erk is indicated. Values were normalized to Erk activity of ctrl-treated EpH4 cells in sparse collagen, n=3. NS indicated “no significance,” as measured by 2-tailed t-test.

FIG. 11. The mammary glands of Mmp14(−/−) have lower Itgb1 expression levels. MGs from (i) Mmp14 (+/−) or (ii) Mmp14 (−/−) mouse were immunostained with Itgb1 (green) and DAPI (red). See FIG. 3a-iv for quantification of Itgb1 intensity. The mammary gland from Mmp14 (−/−) mice showed fewer lipid droplets in the mammary fat tissue. This phenotype might be due to less adipogenesis as reported previously (Chun et al., 2006).

FIG. 12. Immunoprecipitation of Itgb1 detects Mmp14. Endogenous Itgb1 was immunoprecipitated with hamster anti-Itgb1 (Santa Cruz biotechnology) from the EpH4 cell lysate. Immunoprecipitated material was probed for Itgb1 and Mmp14 using rabbit anti-Itgb1 (Santa Cruz biotechnology) or rabbit anti-Mmp14 (Abcam), respectively. Control hamster IgG was purchased from Santa Cruz biotechnology.

FIG. 13. Overexpressing full-length MMP14 or the dCAT/dPEX mutant in Mmp14-silenced EpH4 cells rescues MEC invasion/branching in sparse CL-1 gels. Mmp14-silenced EpH4 cells were infected with lentivirus containing (i) control, (ii) MMP14F-FL, (iii) MMP14F-dCAT or (iv) MMP14F-dCAT/dPEX, respectively. Cells were cultured in 1 mg/ml CL-1 gel. Scale bar: 100 □m.

FIG. 14. MMP14-FL and MMP14-dCAT/dPEX associate with Itgb1, but MMP14dCAT does not. (a) FRET analysis was performed on Mmp14 silenced EpH4 cells. ITGB1 mCypet was expressed as a FRET donor, and mYpet tagged MMP14 mutants were used as a FRET acceptor. Ypet emission signal was detected when Cypet was excited indicating FRET. (i) ITGB1mCypet/MMP14 FL-mYpet, (ii) ITGB1mCypet/dCAT-mYpet and (iii) ITGB1mCypet/dCAT dPEX-mYpet were shown. The heat map indicator is presented to show the intensity of the FRET signal. (iv) Quantification of FRET signals. ˜200 FRET signals were quantified on each condition. Data are mean+/−S.E., (****) indicates p<0.001. (b) Immunoprecipitation between MMP14 mutants and Itgb1. MMP14-FL, dCAT or dCAT/dPEX expressing EpH4 cells were lysed with lysis buffer (1% Brij98, 25 mM HEPES pH 7.4, 150 mM NaCl, protease inhibitors), and immunoprecipitated with anti-FLAG M2 beads (Sigma). Immuno-complexes are used for IP-WB analysis. Blots show FLAG tagged MMP14 mutants (upper) and Itgb1 (lower).

FIG. 15. Mammary gland branching morphogenesis is reduced in Mmp14 (−/−) mouse. (a) The ductal tree in the mammary gland from (i) wild type and (ii) Mmp14KO are shown. Tissue images were captured with confocal microscopy to quantify 3D parameters (Mori et al., 2012). (b) Branch length (i), Branch interval (ii) and Branch points (iii) were analyzed with IMARIS software (Bitplane). N=5. Scale bar: 200 □m.

FIG. 16. Invasion/branching is inhibited by silencing either Mmp14 or Itgb1 in CL-1 gels. Images show Mmp14- or Itgb1-silenced EpH4 cells in dense (3 mg/ml) or sparse (1 mg/ml) CL-1 gels. Short hairpin sequences for Mmp14 or Itgb1 are indicated on the bottom panel and correspond to SEQ ID NOS:52-59. Total RNA was isolated from shRNA treated MECs, and tested to validate knock-down. Sequences #1 and #2 on the list provided more than an 80% knock-down and were used in experiments. The short hairpin sequences for Mmp14 and Itgb1 are indicated.

FIG. 17. The MMP3 hemopexin domain induces altered morphology and invasion in mammary epithelial cells. (A) Schematic representation of engineered constructs: the full-length MMP3 (FL) and two mutants (EA and dPEX). (B) Overexpression of MMP3 and its mutants in SCp2 cells assessed by Western blotting (WB). CM was isolated from cells transduced with each of the MMP3 constructs and the control vector. Flag epitope tag was detected with anti-Flag antibody. Both latent (lat) and activated (act) forms of MMP3 were recognized. (C) MMP3 proteolytic activity of SCp2 cells overexpressing each construct assayed by casein degradation. CM was incubated with a dye-quenching casein substrate (BODIPY TR-X casein). MMP3-mediated degradation of casein generated fluorescent dye-labeled peptides that were monitored over time. Fluorescence intensity is indicated as arbitrary units (AU). (D) Overexpression of MMP3 containing the hemopexin domain induces scattering in SCp2 cells. Scattering ability was evaluated in cells transduced with each construct upon stimulation with epidermal growth factor (EGF). Bars, 20 μm. (E) The presence of the hemopexin domain of MMP3 is required to disrupt adherens junctions. Immunofluorescence images show E-cadherin distribution (green) in cells expressing each construct. Arrows depict areas of cell-cell contact. Nuclei were stained with DAPI (blue). Bars, 10 μm. (F) The MMP3 hemopexin domain induces reorganization of F-actin. Images show F-actin (magenta) and nuclei (DAPI; blue) in each culture. Bars, 10 μm. (G) Quantification of morphological changes in each culture by calculation of the cellular elliptical factor. This is defined as the ratio of the longest (length) to the shortest (width) axis of the cell. The box plot shows the median and the interquartile range, and the whiskers show the extreme values. n=100 cells for each stable cell line. (***) P<0.0001 by Student's t-test. (H) The MMP3 hemopexin domain is required for invasion. Invasiveness in each condition was assayed in Boyden chambers. Results are indicated as mean±SD from three independent experiments (10 bright-field images in 20× magnification were counted). (***) P<0.0001; (*) P<0.05 by Student's t-test.

FIG. 18. Proteomic screen of MMP3-binding partners reveals an extracellular role for HSP90β, ANXA2, and MARCKS in MMP3-driven invasion via the hemopexin domain. (A) Strategy for screening MMP3-binding partners through the hemopexin domain. (B) Selection of MARCKS, ANXA2, and HSP90β from proteomic analysis. (Left) Venn diagram showing the spectrum of proteins detected in FL and/or dPEX Flag-immunoprecipitated samples. (Right) Heat map illustrating the relative difference in abundance of proteins detected in both FL and dPEX but much higher in FL. Proteins were sorted by the highest ratio between FL and dPEX. (C) Co-IP of each mutant shows the association between MMP3 and the selected targets via the hemopexin domain. Flag-tagged MMP3 FL, EA, and dPEX were immunoprecipitated from CM with an anti-Flag antibody and blotted with antibodies for its binding partners. (D-F) Blots showing shRNA-mediated silencing of HSP90β (D), ANXA2 (E), and MARCKS (F) in SCp2 cells overexpressing each of the MMP3 constructs and the control vector. Knockdowns were reproduced using two other shRNAs for each one of the targets (Supplemental Fig. S5A-C,G-I). Nontargeting shRNA was used as negative control. Knockdowns were verified by Western blotting of whole-cell lysates with antibodies specific for each target protein. α-Tubulin was used as loading control. (G-I) Silencing of HSP90β, ANXA2, and MARCKS reduces MMP3-driven invasion in SCp2 cells when the hemopexin domain of MMP3 is present. SCp2 cells were cotransduced with each of the MMP3 constructs and either nontargeting shRNA or shRNAs selectively targeting HSP90β (G), ANXA2 (H), or MARCKS (I). SCp2 parental cells were treated with CM from each engineered cell line and assayed for invasiveness in Boyden chambers. Parental cells treated with CM from SCp2 cells expressing each of the MMP3 constructs and the control vector (untreated CM) were used as control. Results are expressed as mean±SD from three independent experiments (10 bright-field images in 20× magnification were counted in each experiment). (**) P<0.001; (*) P<0.05 by Student's t-test. The biological effects of shRNA-mediated knockdowns were reproduced with two other shRNAs for each of the three interacting proteins (Supplemental Fig. S5D-F,J-L).

FIG. 19. The hemopexin domain of MMP3 is necessary to direct the epithelial invasion of mammary organoids in 3D Col-1 gels even without the proteolytic activity. (A) Schematic representation of primary mammary organoid preparation and culture in 3D Col-1 gels. (B) Overexpression of MMP3 containing the hemopexin domain enhances the invasion of mammary organoids in Col-1. Images of maximum intensity projection of mammary organoids transduced with each of the MMP3 constructs as well as the control vector and cultured in 3 mg/mL Col-1 gels for 3 d. Organoids invaded and branched only in the presence of the growth factor (TGFα). Structures were stained for F-actin (red) and nuclei (DAPI; blue). Image background was pseudo-colored in gray. Bars, 100 μm. (C) The presence of the hemopexin domain of MMP3 increases the number of extending processes developed from each organoid invading through Col-1 (150 organoids were counted per culture). (***) P<0.0001; (**) P<0.001 by Student's t-test. (D) The size of the spatial network per organoid is increased by overexpression of MMP3 containing the hemopexin domain. The spatial network per organoid is defined as the sum of the length of all the extending processes of an organoid (50 organoids were counted per culture). (***) P<0.0001 by Student's t-test.

FIG. 20. Extracellular HSP90β modulates MMP3 function in invasion and branching of mammary epithelial organoids. (A) Recombinant HSP90β added to the medium increases the invasiveness of mammary organoids expressing MMP3. Images of maximum intensity projection from confocal z-stacks of mammary organoids overexpressing FL-MMP3 or control vector embedded in 3 mg/mL Col-1 gels. Organoids were cultured for 3 d in the presence or absence of a recombinant HSP90β. Structures were stained for F-actin (red) and nuclei (DAPI; blue). Image background was pseudo-colored in gray. Bars, 100 μm. (B) Inhibition of extracellular HSP90β abolishes the branching ability of mammary organoids. Images of maximum intensity projection from confocal z-stacks of mammary organoids overexpressing FL MMP3 or control vector embedded in 3 mg/mL Col-1 gels. Organoids were cultured for 3 d with a function-blocking antibody against HSP90β or a control IgG. Structures were stained for F-actin (red) and nuclei (DAPI; blue). Image background was pseudo-colored in gray. Bars, 100 μm. (C,D) Quantification of invasion and branching by measuring the spatial network per organoid (50 organoids were counted per culture). (***) P<0.0001; (*) P<0.05 by Student's t-test.

FIG. 21. Scheme of the essential role of extracellular HSP90b in the modulation of MMP3-driven invasion and branching in mammary organoids. When organoids from the mammary gland are embedded within 3D gels of Col-1, they undergo invasion and branching morphogenesis upon addition of growth factors. The small endogenous MMP3 activity present in the organoids provides them a baseline of branching to which we could compare the exogenous constructs. The insertion of exogenous MMP3 induces a hyperbranched phenotype only when the hemopexin domain is present. This region mediates the extracellular interaction with HSP90b and is critical for the invasive function of MMP3. Recombinant HSP90b added extracellularly enables the secreted MMP3 to induce the most exuberant branched structures. Conversely, blocking of extracellular HSP90b with inhibitory antibodies added to the medium abolishes branching ability.

FIG. 22. The endogenous and exogenous levels of MMP3 are comparable in all engineered cell lines. (A) Expression of endogenous and exogenous MMP3 in SCp2 cells assessed by Western blotting (WB). Whole cell lysates were isolated from cells transduced with each of the MMP3 constructs and the control vector. Flag epitope tag was detected with anti-Flag antibody, and endogenous MMP3 was detected with an antibody specific for murine MMP3. α-Tubulin was used as loading control. (B) Quantification of the ratio between exogenous and endogenous MMP3 in each culture. Results are indicated as mean±SD from three independent experiments.

FIG. 23. Shedding of E-cadherin is dependent on MMP3 proteolytic activity, and the hemopexin domain of MMP3 is required to induce invasion and elongated morphology also in EpH4 cells. (A) E-cadherin is a substrate for MMP3. Western blotting (WB) showing soluble fragments of E-cadherin (sE-cadherin) detected in CM from SCp2 cells overexpressing distinct MMP3 constructs and control vector. Total E-cadherin and α-tubulin from whole cell lysates were used as loading controls. (B) Overexpression of MMP3 and its mutants in EpH4 cells assessed by WB. CM was isolated from cells transduced with each of the MMP3 constructs as well as the control vector. Flag epitope tag was detected with anti-Flag antibody. (C) MMP3 hemopexin domain directs signaling for invasion. Invasiveness in each condition was assayed in Boyden chambers. Results are indicated as mean±SD from three independent experiments (10 bright field images in 20× magnification were counted). (**) P<0.001 by Student's t-test. (D) MMP3 hemopexin domain induces reorganization of F-actin. Images show F-actin (magenta) and nuclei (DAPI; blue) in each culture. Bars, 10 μm. (E) Quantification of morphological changes in each culture by calculation of the cellular elliptical factor. The box plot shows the median and the interquartile range, and the whiskers show the extreme values. n=100 cells for each stable cell line. (***) P<0.0001 by Student's t-test.

FIG. 24. CM from cells overexpressing each of the MMP3 constructs is sufficient to induce morphological and functional changes in parental SCp2 cells. (A) CM is sufficient to induce reorganization of F-actin in parental SCp2 cells. Images show F-actin (magenta) and nuclei (DAPI; blue) in each culture. Bars, 10 μm. (B) Quantification of morphological changes in each culture by calculation of the cellular elliptical factor. The box shows the median and the interquartile range, and the whiskers show the extreme values. n=100 cells per culture. (***) P<0.0001 by Student's t-test. (C) CM is sufficient to trigger invasion in parental SCp2 cells. Invasiveness in each condition was assayed in Boyden chambers. Results are expressed as mean±SD. from three independent experiments (10 bright field images in 20× magnification were counted). (***) P<0.0001; (*) P<0.05 by Student's t-test.

FIG. 25. Proteins identified by mass spectrometry as interacting with MMP3 in the extracellular milieu. (Left) Heat map showing the relative difference in protein abundance in FL vs. control, dPEX vs. control and FL vs. dPEX Flag-immunoprecipitated samples. Proteins are sorted by the ratio of FL and dPEX. (Right) Complete list of the identified proteins. Targets selected for validation and further studies are highlighted.

FIG. 26. The biological effects of shRNA-mediated knockdowns of HSP90β, ANXA2 and MARCKS were reproduced with two other shRNAs for each of those proteins. Blots showing shRNA-mediated silencing of HSP90β (A,G), ANXA2 (B,H) and MARCKS (C,I) in SCp2 cells overexpressing each of the MMP3 constructs and the control vector. Nontargeting shRNA was used as negative control. Knockdowns were verified by Western blotting (WB) of whole cell lysates with antibodies specific for each target protein. α-Tubulin was used as loading control. (D-F, J-L) Silencing of HSP90β, ANXA2 and MARCKS reduces MMP3-driven invasion in SCp2 cells when the hemopexin domain of MMP3 is present. SCp2 cells were cotransduced with each of the MMP3 constructs and either nontargeting shRNA or shRNAs selectively targeting HSP90β (D,J), ANXA2 (E,K) or MARCKS (F,L). SCp2 parental cells were treated with CM from each engineered cell line and assayed for invasiveness in Boyden chambers. Parental cells treated with CM from SCp2 cells expressing each of the MMP3 constructs and the control vector (untreated CM) were used as control. Results are expressed as mean±SD from three independent experiments (10 bright field images in 20× magnification were counted in each experiment). (**) P<0.001; (*) P<0.05 by Student's t-test.

FIG. 27. Co-IP of HSP90β protein complexes confirms the extracellular association of MMP3 and HSP90β in reverse. HSP90β protein complexes were immunoprecipitated from CM from control SCp2 cells with a rabbit anti-HSP90β antibody, and blotted with antibodies for HSP90β, MMP3, ANXA2 and MARCKS. Control rabbit-IgG and plain protein G sepharose beads were used as controls.

FIG. 28. Extracellular HSP90β regulates MMP3-driven invasion in a dose-dependent manner when the hemopexin domain is present. (A) HSP90β enhances the invasiveness of SCp2 cells overexpressing MMP3 constructs containing the hemopexin domain. Cell invasiveness of FL, EA, dPEX-overexpressing SCp2 and control vector cells cultured in the presence of increasing doses of recombinant HSP90β. Untreated cells were used as a control. (B) Inhibition of HSP90β reduces invasiveness of SCp2 cells overexpressing MMP3 constructs containing the hemopexin domain. Cell invasiveness of FL, EA, dPEX-overexpressing SCp2 and control vector cells cultured in the presence of increasing doses of a cell-permeable HSP90β inhibitor. Cells cultured with vehicle DMSO and untreated cells were used as controls. (C) Blocking of extracellular HSP90β decreases invasiveness of SCp2 cells transduced with MMP3 constructs containing the hemopexin domain. Cell invasiveness of FL, EA, dPEX-overexpressing SCp2 and control vector cells cultured in the presence of a cell-permeable HSP90β inhibitor or a function-blocking antibody against HSP90β. Untreated cells and rabbit IgG were used as controls. Invasiveness from experiments in (A-C) was assayed in Boyden chambers. Results are expressed as the mean±SD from three independent experiments (10 bright field images in 20× magnification were counted). (**) P<0.001; (*) P<0.05 by Student's t-test.

FIG. 29. The hemopexin domain of MMP3 is necessary to direct invasion of clustered EpH4 cells in 3D Col-1 gels even without the proteolytic activity. (A) Schematic representation of cell cluster preparation and culture in 3D Col-1 gels. (B) Images of maximum intensity projection of EpH4 cell clusters transduced with each of the MMP3 constructs as well as the control vector, and cultured in 3 mg/mL Col-1 for 4 days. EpH4 clusters invaded and branched only in the presence of the growth factor (bFGF). Structures were stained for F-actin (red) and nuclei (DAPI; blue). Image background was pseudo-colored in gray. Bars, 100 μm.

FIG. 30. The hemopexin domain of MMP3 is sufficient for epithelial invasion, but the proteolytic activity is still required for the formation of branches. (A) Images of maximum intensity projection of mammary organoids transduced with each of the MMP3 constructs as well as the control vector, and cultured in 3 mg/mL Col-1 gels for 3 d. Organoids were cultured in the presence of the growth factor (TGFα) and increasing doses of a peptide that was shown previously to inhibit MMP3 proteolytic activity specifically and significantly. Structures were stained for F-actin (red) and nuclei (DAPI; blue). Image background was pseudo-colored in gray. Bars, 100 μm. (B) Quantification of invasion and branching by measuring the spatial network per organoid (50 organoids were counted per culture). (***) P<0.0001; (**) P<0.001; (*) P<0.05 by Student's t-test.

FIG. 31. shRNA-mediated knockdown of MMP3 reduces invasion and branching significantly in control organoids. (A) Images of maximum intensity projection of control mammary organoids infected with either nontargeting shRNA or shRNA selectively targeting MMP3, and cultured in 3 mg/mL Col-1 gels for 3 d. Organoids were cultured in the presence of the growth factor (TGFα). Structures were stained for F-actin (red) and nuclei (DAPI; blue). Image background was pseudo-colored in gray. Bars, 100 μm. (B) Blots showing silencing of MMP3 using two distinct shRNAs. Knockdown was verified by western blotting (WB) of whole cell lysates with an antibody specific for murine MMP3. α-Tubulin was used as loading control. (C) Silencing MMP3 decreases the size of the spatial network per organoid. Quantification of invasion and branching was performed by measuring the spatial network per organoid (50 organoids were counted per culture). (***) P<0.0001 by Student's t-test.

### DETAILED DESCRIPTION OF THE INVENTION

Epithelial cell invasion through the extracellular matrix (ECM) is a critical step in branching morphogenesis. The mechanisms by which the mammary epithelium integrates cues from the ECM with intracellular signaling to coordinate invasion through the stroma to make the mammary tree are poorly understood.

In one embodiment, because the cell membrane-bound matrix metalloproteinase 14 (Mmp14) is known to play a key role in cancer cell invasion, we hypothesized that it could also be centrally involved in integrating signals as mammary epithelial cells (MECs) navigate the collagen-1 (CL-1)-rich stroma of the mammary gland (MG). Expression studies in nulliparous mice that carry a NLS-lacZ transgene downstream of the Mmp14 promoter revealed that Mmp14 is expressed in MECs at the tips of the branches. Using both mammary organoids and 3D organotypic cultures, we show that MMP activity is necessary for invasion within a concentration of dense CL-1 (3 mg/ml), but dispensable for MEC branching in sparse CL-1 (1 mg/ml). Surprisingly, however, Mmp14 without its catalytic activity was still necessary for branching. Silencing Mmp14 prevented cell invasion through CL-1 and disrupted branching altogether; it also reduced integrin-β1 (Itgb1) levels and attenuated MAPK signaling, disrupting Itgb1-dependent invasion/branching within CL-1 gels. FRET imaging revealed that Mmp14 associates directly with Itgb1. We identified the hemopexin domain of MMP14, which is required for modulating the levels of Itgb1, MEC signaling and the rate of invasion within CL-1. These results shed light on hitherto undescribed non-proteolytic activities of MMP14 that are necessary for Itgb1-dependent biochemical and mechanical signals that regulate branching in the mammary epithelium.

We describe herein that domains of matrix metalloproteinases, such as MMP-3, MMP-9 and MMP-14, other than its catalytic domain may be targets for controlling cellular invasion in cancer. These non-catalytic domains may be helpful in controlling metastasis and cell migration. Thus, in some embodiments, this disclosure provides inhibitors to and proteins that bind MMPs such as MMP-3, MMP-9 and MMP-14, and methods of identifying and using such inhibitors. For example, inhibitors to and proteins that bind to MMP-14 are herein referred to as “MMP-14 inhibitors”. These inhibitors include but are not limited to antisense and nucleotide inhibitors, polynucleotides, peptides and peptide mimetics, polypeptides, antibodies and antibody fragments (e.g., primate antibodies and Ribs, especially human antibodies and Fabs), and small molecules or drugs, that bind to and or inhibit MMPs, such as MMP-14 (e.g., human MMP-14). The MMP inhibitors can be used in the treatment of diseases, particularly human disease, such as cancer, in which excess or inappropriate activity of the non-catalytic domain of the MMP is observed or featured.

The present application hereby incorporates by reference Devy et al, U.S. Pub No. 20120107231 in its entirety, which describes metalloproteinase binding proteins use of such proteins, including antibody proteins which bind to MMP14 catalytic domains. Also incorporated by reference D'Angelo et al, U.S. Pub. No. 20120088722 in its entirety, and provides compounds for disrupting the binding of a matrix metalloprotease (MMP) protein to a substrate protein at an interaction site other than the protease catalytic site and the compounds are preferably polypeptide fragments of the hemopexin-like domain of the MMP.

MMP-14 is encoded by a gene designated as MMP14, matrix metalloproteinase-14 precursor. Synonyms for MMP-14 include matrix metalloproteinase 14 (membrane-inserted), membrane-type-1 matrix metalloproteinase, membrane-type matrix metalloproteinase 1, MMP-14, MMP-X1, MT1MMP, MT1-MMP, MTMMP1, MT-MMP 1.

Membrane-type MMPs have similar structures, including a signal peptide, a prodomain, a catalytic domain, a hinge region, and a hemopexin domain (Wang, et al., 2004, J Biol Chem 279151148-55). According to SwissProt entry P50281, the signal sequence of MMP-14 precursor includes amino acid residues 1-20. The pro-peptide includes residues 21-111. Cys93 is annotated as a possible cysteine switch. Residues 112 through 582 make up the mature, active protein. The catalytic domain includes residues 112-317. The non-catalytic domain of MMP14 comprised of residues 318-562. The hemopexin domains in the non-catalytic domain includes residues 318-523. The transmembrane segment comprises residues 542 through 562.

An exemplary amino acid sequence of human MMP14 is amino-acid sequence of human MMP14 (SEQ ID NO: 2: Genbank Accession No. CAA83372.1) shown here:

MSPAPRPPRCLLLPLLTLGTALASLGSAQSSSFSPEAWLQQYGYLPPGDL
RTHTQRSPQSLSAAIAAMQKFYGLQVTGKADADTMKAMRRPRCGVPDKFG
AEIKANVRRKRYAIQGLKWQHNEITFCTQNYTPKVGEYATYEAIRKAFRV
WESATPLRFREVPYAYIREGHEKQADIMIFFAEGFHGDSTPFDGEGGFLA
HAYFPGPNIGGDTHFDSAEPWTVRNEDLNGNDIFLVAVHELGHALGLEHS
SDPSAIMAPFYQWMDTENFVLPDDDRRGIQQLYGGESGFPTKMPPQPRTT
SRPSVPDKPKNPTYGPNICDGNFDTVAMLRGEMFVFKERWFWRVRNNQVM
DGYPMPIGQFWRGLPASINTAYERKDGKFVFFKGDKHWVFDEASLEPGYP
KHIKELGRGLPTDKIDAALFWMPNGKTYFFRGNKYYRFNEELRAVDSEYP
KNIKVWEGIPESPRGSFMGSDEVFTYFYKGNKYWKFNNQKLKVEPGYPKS
ALRDWMGCPSGGRPDEGTEEETEVIIIEVDEEGGGAVSAAAVVLPVLLLL
LVLAVGLAVFFFRRHGTPRRLLYCQRSLLDKV

An exemplary amino acid sequence of mouse MMP14 is amino-acid sequence of mouse MMP14 (SEQ ID NO: 4: GenBank Accession No. NP_{—}032634.2.) shown here:

MSPAPRPSRSLLLPLLTLGTALASLGWAQGSNFSPEAWLQQYGYLPPGDL
RTHTQRSPQSLSAAIAAMQKFYGLQVTGKADLATMMAMRRPRCGVPDKFG
TEIKANVRRKRYAIQGLKWQHNEITFCIQNYTPKVGEYATFEAIRKAFRV
WESATPLRFREVPYAYIREGHEKQADIMILFAEGFHGDSTPFDGEGGFLA
HAYFPGPNIGGDTHFDSAEPWTVQNEDLNGNDIFLVAVHELGHALGLEHS
NDPSAIMSPFYQWMDTENFVLPDDDRRGIQQLYGSKSGSPTKMPPQPRTT
SRPSVPDKPKNPAYGPNICDGNFDTVAMLRGEMFVFKERWFWRVRNNQVM
DGYPMPIGQFWRGLPASINTAYERKDGKFVFFKGDKHWVFDEASLEPGYP
KHIKELGRGLPTDKIDAALFWMPNGKTYFFRGNKYYRFNEEFRAVDSEYP
KNIKVWEGIPESPRGSFMGSDEVFTYFYKGNKYWKFNNQKLKVEPGYPKS
ALRDWMGCPSGRRPDEGTEEETEVIIIEVDEEGSGAVSAAAVVLPVLLLL
LVLAVGLAVFFFRRHGTPKRLLYCQRSLLDKV

These exemplary hMMP-14 and mMMP-14 sequences are identical at 558 of 580 positions, about 96.2% identity. Despite a relatively high degree of similarity, their activity toward different substrates, including proMMP-2 and type I collagen, varies (Wang, et al., 2004, J Biol Chem, 279:51148-55).

An exemplary nucleotide sequence of Homo sapiens matrix metallopeptidase 3 (stromelysin 1, progelatinase) (MMP3), mRNA (SEQ ID NO:5; GenBank NM_{—}002422.3 GI:73808272) shown here:

```
      1 ctacaaggag gcaggcaaga cagcaaggca tagagacaac atagagctaa gtaaagccag
    61 tggaaatgaa gagtcttcca atcctactgt tgctgtgcgt ggcagtttgc tcagcctatc
  121 cattggatgg agctgcaagg ggtgaggaca ccagcatgaa ccttgttcag aaatatctag
  181 aaaactacta cgacctcaaa aaagatgtga aacagtttgt taggagaaag gacagtggtc
  241 ctgttgttaa aaaaatccga gaaatgcaga agttccttgg attggaggtg acggggaagc
  301 tggactccga cactctggag gtgatgcgca agcccaggtg tggagttcct gatgttggtc
  361 acttcagaac ctttcctggc atcccgaagt ggaggaaaac ccaccttaca tacaggattg
  421 tgaattatac accagatttg ccaaaagatg ctgttgattc tgctgttgag aaagctctga
  481 aagtctggga agaggtgact ccactcacat tctccaggct gtatgaagga gaggctgata
  541 taatgatctc ttttgcagtt agagaacatg gagactttta cccttttgat ggacctggaa
  601 atgttttggc ccatgcctat gcccctgggc cagggattaa tggagatgcc cactttgatg
  661 atgatgaaca atggacaaag gatacaacag ggaccaattt atttctcgtt gctgctcatg
  721 aaattggcca ctccctgggt ctctttcact cagccaacac tgaagctttg atgtacccac
  781 tctatcactc actcacagac ctgactcggt tccgcctgtc tcaagatgat ataaatggca
  841 ttcagtccct ctatggacct ccccctgact cccctgagac ccccctggta cccacggaac
  901 ctgtccctcc agaacctggg acgccagcca actgtgatcc tgctttgtcc tttgatgctg
  961 tcagcactct gaggggagaa atcctgatct ttaaagacag gcacttttgg cgcaaatccc
1021 tcaggaagct tgaacctgaa ttgcatttga tctcttcatt ttggccatct cttccttcag
1081 gcgtggatgc cgcatatgaa gttactagca aggacctcgt tttcattttt aaaggaaatc
1141 aattctgggc tatcagagga aatgaggtac gagctggata cccaagaggc atccacaccc
1201 taggtttccc tccaaccgtg aggaaaatcg atgcagccat ttctgataag gaaaagaaca
1261 aaacatattt ctttgtagag gacaaatact ggagatttga tgagaagaga aattccatgg
1321 agccaggctt tcccaagcaa atagctgaag actttccagg gattgactca aagattgatg
1381 ctgtttttga agaatttggg ttcttttatt tctttactgg atcttcacag ttggagtttg
1441 acccaaatgc aaagaaagtg acacacactt tgaagagtaa cagctggctt aattgttgaa
1501 agagatatgt agaaggcaca atatgggcac tttaaatgaa gctaataatt cttcacctaa
1561 gtctctgtga attgaaatgt tcgttttctc ctgcctgtgc tgtgactcga gtcacactca
1621 agggaacttg agcgtgaatc tgtatcttgc cggtcatttt tatgttatta cagggcattc
1681 aaatgggctg ctgcttagct tgcaccttgt cacatagagt gatctttccc aagagaaggg
1741 gaagcactcg tgtgcaacag acaagtgact gtatctgtgt agactatttg cttatttaat
1801 aaagacgatt tgtcagttat tttatctt
```

An exemplary protein sequence of Homo sapiens matrix metallopeptidase 3 (stromelysin 1, progelatinase) (MMP3) (SEQ ID NO: 6; GenBank Accession No. NP_{—}002413.1 GI:4505217) shown here:

```
    1 MKSLPILLLL CVAVCSAYPL DGAARGEDTS MNLVQKYLEN YYDLKKDVKQ FVRRKDSGPV
  61 VKKIREMQKF LGLEVTGKLD SDTLEVMRKP RCGVPDVGHF RTFPGIPKWR KTHLTYRIVN
121 YTPDLPKDAV DSAVEKALKV WEEVTPLTFS RLYEGEADIM ISFAVREHGD FYPFDGPGNV
181 LAHAYAPGPG INGDAHFDDD EQWTKDTTGT NLFLVAAHEI GHSLGLFHSA NTEALMYPLY
241 HSLTDLTRFR LSQDDINGIQ SLYGPPPDSP ETPLVPTEPV PPEPGTPANC DPALSFDAVS
301 TLRGEILIFK DRHFWRKSLR KLEPELHLIS SFWPSLPSGV DAAYEVTSKD LVFIFKGNQF
361 WAIRGNEVRA GYPRGIHTLG FPPTVRKIDA AISDKEKNKT YFFVEDKYWR FDEKRNSMEP
421 GFPKQIAEDF PGIDSKIDAV FEEFGFFYFF TGSSQLEFDP NAKKVTHTLK SNSWLNC
```

The four hemopexin domains in MMP3 are described as corresponding to amino acid residues 287-336 Hemopexin 1; residues 337-383 Hemopexin 2; residues 385-433 Hemopexin 3; and residues 434-477 Hemopexin 4

An exemplary nucleotide sequence of Homo sapiens matrix metallopeptidase 9 (gelatinase B, 92 kDa gelatinase, 92 kDa type IV collagenase) (MMP9), mRNA (SEQ ID NO: 7; GenBank NM_{—}004994.2 GI:74272286) shown here:

```
      1 agacacctct gccctcacca tgagcctctg gcagcccctg gtcctggtgc tcctggtgct
    61 gggctgctgc tttgctgccc ccagacagcg ccagtccacc cttgtgctct tccctggaga
  121 cctgagaacc aatctcaccg acaggcagct ggcagaggaa tacctgtacc gctatggtta
  181 cactcgggtg gcagagatgc gtggagagtc gaaatctctg gggcctgcgc tgctgcttct
  241 ccagaagcaa ctgtccctgc ccgagaccgg tgagctggat agcgccacgc tgaaggccat
  301 gcgaacccca cggtgcgggg tcccagacct gggcagattc caaacctttg agggcgacct
  361 caagtggcac caccacaaca tcacctattg gatccaaaac tactcggaag acttgccgcg
  421 ggcggtgatt gacgacgcct ttgcccgcgc cttcgcactg tggagcgcgg tgacgccgct
  481 caccttcact cgcgtgtaca gccgggacgc agacatcgtc atccagtttg gtgtcgcgga
  541 gcacggagac gggtatccct tcgacgggaa ggacgggctc ctggcacacg cctttcctcc
  601 tggccccggc attcagggag acgcccattt cgacgatgac gagttgtggt ccctgggcaa
  661 gggcgtcgtg gttccaactc ggtttggaaa cgcagatggc gcggcctgcc acttcccctt
  721 catcttcgag ggccgctcct actctgcctg caccaccgac ggtcgctccg acggcttgcc
  781 ctggtgcagt accacggcca actacgacac cgacgaccgg tttggcttct gccccagcga
  841 gagactctac acccaggacg gcaatgctga tgggaaaccc tgccagtttc cattcatctt
  901 ccaaggccaa tcctactccg cctgcaccac ggacggtcgc tccgacggct accgctggtg
  961 cgccaccacc gccaactacg accgggacaa gctcttcggc ttctgcccga cccgagctga
1021 ctcgacggtg atggggggca actcggcggg ggagctgtgc gtcttcccct tcactttcct
1081 gggtaaggag tactcgacct gtaccagcga gggccgcgga gatgggcgcc tctggtgcgc
1141 taccacctcg aactttgaca gcgacaagaa gtggggcttc tgcccggacc aaggatacag
1201 tttgttcctc gtggcggcgc atgagttcgg ccacgcgctg ggcttagatc attcctcagt
1261 gccggaggcg ctcatgtacc ctatgtaccg cttcactgag gggcccccct tgcataagga
1321 cgacgtgaat ggcatccggc acctctatgg tcctcgccct gaacctgagc cacggcctcc
1381 aaccaccacc acaccgcagc ccacggctcc cccgacggtc tgccccaccg gaccccccac
1441 tgtccacccc tcagagcgcc ccacagctgg ccccacaggt cccccctcag ctggccccac
1501 aggtcccccc actgctggcc cttctacggc cactactgtg cctttgagtc cggtggacga
1561 tgcctgcaac gtgaacatct tcgacgccat cgcggagatt gggaaccagc tgtatttgtt
1621 caaggatggg aagtactggc gattctctga gggcaggggg agccggccgc agggcccctt
1681 ccttatcgcc gacaagtggc ccgcgctgcc ccgcaagctg gactcggtct ttgaggagcg
1741 gctctccaag aagcttttct tcttctctgg gcgccaggtg tgggtgtaca caggcgcgtc
1801 ggtgctgggc ccgaggcgtc tggacaagct gggcctggga gccgacgtgg cccaggtgac
1861 cggggccctc cggagtggca gggggaagat gctgctgttc agcgggcggc gcctctggag
1921 gttcgacgtg aaggcgcaga tggtggatcc ccggagcgcc agcgaggtgg accggatgtt
1981 ccccggggtg cctttggaca cgcacgacgt cttccagtac cgagagaaag cctatttctg
2041 ccaggaccgc ttctactggc gcgtgagttc ccggagtgag ttgaaccagg tggaccaagt
2101 gggctacgtg acctatgaca tcctgcagtg ccctgaggac tagggctccc gtcctgcttt
2161 ggcagtgcca tgtaaatccc cactgggacc aaccctgggg aaggagccag tttgccggat
2221 acaaactggt attctgttct ggaggaaagg gaggagtgga ggtgggctgg gccctctctt
2281 ctcacctttg ttttttgttg gagtgtttct aataaacttg gattctctaa cctttaaaaa
2341 aaaaaaaaaa aaaaaaaaaa aaaaaaaaaa aaaaaaaaaa aaaaaaa
```

An exemplary protein sequence of matrix metalloproteinase-9 preproprotein [Homo sapiens] (SEQ ID NO: 8; GenBank Accession No. NP_{—}004985.2 GI:74272287) shown here:

MSLWQPLVLVLLVLGCCFAAPRQRQSTLVLFPGDLRTNLTDRQL
AEEYLYRYGYTRVAEMRGESKSLGPALLLLQKQLSLPETGELDSATLKA
MRTPRCGVPDLGRFQTFEGDLKWHHHNITYWIQNYSEDLPRAVIDDAFA
RAFALWSAVTPLTFTRVYSRDADIVIQFGVAEHGDGYPFDGKDGLLAHA
FPPGPGIQGDAHFDDDELWSLGKGVVVPTRFGNADGAACHFPFIFEGRS
YSACTTDGRSDGLPWCSTTANYDTDDRFGFCPSERLYTQDGNADGKPCQ
FPFIFQGQSYSACTTDGRSDGYRWCATTANYDRDKLFGFCPTRADSTVM
GGNSAGELCVFPFTFLGKEYSTCTSEGRGDGRLWCATTSNFDSDKKWGF
CPDQGYSLFLVAAHEFGHALGLDHSSVPEALMYPMYRFTEGPPLHKDDV
NGIRHLYGPRPEPEPRPPTTTTPQPTAPPTVCPTGPPTVHPSERPTAGP
TGPPSAGPTGPPTAGPSTATTVPLSPVDDACNVNIFDAIAEIGNQLYLF
KDGKYWRFSEGRGSRPQGPFLIADKWPALPRKLDSVFEERLSKKLFFFS
GRQVWVYTGASVLGPRRLDKLGLGADVAQVTGALRSGRGKMLLFSGRRL
WRFDVKAQMVDPRSASEVDRMFPGVPLDTHDVFQYREKAYFCQDRFYWR
VSSRSELNQVDQVGYVTYDILQCPED

An exemplary protein sequence of matrix metalloproteinase-9 [Homo sapiens] (MMP-9) (SEQ ID NO:9; GenBank Accession No. CAC07541.1 GI:9997653) described in WO 9957315-A2 11 Nov. 1999 and shown here:

```
    1 mslwqplvlv llvlgccfaa prqrqstlvl fpgdlrtnlt drqlaeeyly rygytrvaem
  61 rgeskslgpa llllqkqlsl petgeldsat lkamrtprcg vpdlgrfqtf egdlkwhhhn
121 itywiqnyse dlpravidda farafalwsa vtpltftrvy srdadiviqf gvaehgdgyp
181 fdgkdgllah afppgpgiqg dahfdddelw slgkgvvvpt rfgnadgaac hfpfifegrs
241 ysacttdgrs dglpwcstta nydtddrfgf cpserlytrd gnadgkpcqf pfifqgqsys
301 acttdgrsdg yrwcattany drdklfgfcp tradstvmgg nsagelcvfp ftflgkeyst
361 ctsegrgdgr lwcattsnfd sdkkwgfcpd qgyslflvaa hefghalgld hssvpealmy
421 pmyrftegpp lhkddvngir hlygprpepe prppttttpq ptapptvcpt gpptvhpser
481 ptagptgpps agptgpptag pstattvpls pvddacnvni fdaiaeignq lylfkdgkyw
541 rfsegrgsrp qgpfliadkw palprkldsv feeplskklf ffsgrqvwvy tgasvlgprr
601 ldklglgadv aqvtgalrsg rgkmllfsgr rlwrfdvkaq mvdprsasev drmfpgvpld
661 thdvfqyrek ayfcqdrfyw rvssrselnq vdqvgyvtyd ilqcped
```

In one embodiment, the MMP inhibitor is a small interference RNA (siRNA). Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection. siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth et al. (2003) Antisense Nucleic Acid Drug Dev. 13(2):83-105; Reynolds et al. (2004) Nat Biotechnol. 22(3):326-30 and Ui-Tei et al., (2004) Nucleic Acids Res. 32(3):936-48, which are hereby incorporated by reference.

Other tools for constructing siRNA sequences are web tools such as the siRNA Target Finder and Construct Builder available from GenScript (website for genscript.com), Oligo Design and Analysis Tools from Integrated DNA Technologies (website for idtdna.com/SciTools/SciTools.aspx), or siDESIGN TM Center from Dharmacon, Inc. (website for design.dharmacon.com/defaultaspx?source=0>). siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In another embodiment, web-based siRNA designing tools from Genescript (URL:=<http://www.genscript.com/rnai.html#design>) may be used to design siRNA sequences that target MMP14 since. Such tools typically provide the top candidate siRNA sequence and also perform BLAST screening (Altschul et al. (1990) “Basic local alignment search tool.” J. Mol. Biol. 215:403-410) on each resulting siRNA sequence.

In another embodiment, the MMP-14 inhibitor is an antisense oligonucleotide. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the MMP-14 oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

These inhibitors include but are not limited to antisense and nucleotide inhibitors, polynucleotides, peptides and peptide mimetics, polypeptides, antibodies and antibody fragments (e.g., primate antibodies and Ribs, especially human antibodies and Fabs), and small molecules or drugs, that bind to and or inhibit MMPs such as MMP-3, MMP-9 or MMP-14 (e.g., human MMP-14).

In another embodiment, aptamer sequences which bind to specific RNA or DNA sequences can be made. Aptamer sequences can be isolated through methods as known in the art.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul et al. (1990) J. Mol. Biol. 215:403-410), and there is nucleotide sequence identity in at least about 80%, preferably at least about 90%, and more preferably at least about 95-98% of the nucleotide bases.

In another embodiment, MMP inhibitors such as siRNA MMP14 inhibitors described herein can also be expressed recombinantly. In general, the nucleic acid sequences encoding MMP inhibitors such as the siRNA MMP inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)). Nucleic acids encoding sequences of MMP inhibitors can also be isolated from expression libraries using antibodies as probes.

In some embodiments, the MMP inhibitor is an antibody (e.g., a polyclonal or monoclonal antibody) that specifically binds and/or inhibits the MMP which can be used using methods known in the art and may be used therapeutically as well. MMP specific antibodies can be made by a number of methods known in the art.

Since synthesized peptides are not always immunogenic on their own, the peptides are conjugated to a carrier protein before use. Appropriate carrier proteins include, but are not limited to, Keyhole limpet hemacyanin (KLH), bovine serum albumin (BSA) and ovalbumin (OVA). The conjugated peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal MMP antibodies can be purified by passing the harvested antibodies through an affinity column. However, monoclonal antibodies are preferred over polyclonal antibodies and can be generated according to standard methods known in the art of creating an immortal cell line which expresses the antibody.

Nonhuman antibodies are highly immunogenic in human thus limiting their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. (Princeton, N.J.). “HuMAb-Mouse” is a strain of transgenic mice that harbors the entire human immunoglobin (Ig) loci and thus can be used to produce fully human monoclonal MMP14 antibodies.

Immunoblotting using the specific antibodies of the invention with MMP sequence should not produce a detectable signal at preferably 0.5-10 fold molar excess (relative to the MMP detection), more preferably at 50 fold molar excess and most preferably no signal is detected at even 100 fold molar excess.

Substantially identical nucleic acids encoding sequences of MMP inhibitors can be isolated using nucleic acid probes and oligonucleotides under stringent hybridization conditions, by screening libraries. Alternatively, expression libraries can be used to clone these sequences, by detecting expressed homologues immunologically with antisera or purified antibodies made against the core domain of nucleic acids encoding MMP inhibitor sequences.

Gene expression of matrix metalloproteinases like MMP-3, MMP-9 and MMP-14 can also be analyzed by techniques known in the art, e.g., reverse transcription and amplification of mRNA, isolation of total RNA or poly A+RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding MMPs, MMP inhibitors such as the siRNA MMP14 inhibitor and related nucleic acid sequence homologues, one typically subclones a sequence (e.g., nucleic acid sequences encoding MMP14 and MMP14 inhibitors such as the siRNA MMP14 inhibitor) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding MMP inhibitors such as the siRNA MMP inhibitor or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant MMP14 inhibitors peptides to provide convenient methods of isolation, e.g., His tags. In some case, enzymatic cleavage sequences (e.g., Met-(His)g-Ile-Glu-Gly-Arg which form the Factor Xa cleavage site) are added to the recombinant MMP14 inhibitor peptides. Bacterial expression systems for expressing the MMP14 inhibitor peptides and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of MMP inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing MMP inhibitor peptides and nucleic acids.

After the expression vector is introduced into the cells, the transfected cells are cultured under conditions favoring expression of MMP inhibitors such as the siRNA MMP inhibitor and related nucleic acid sequence homologues.

### Methods of Treatment

In some embodiments, the invention provides methods of treating disorders associated with MMP induced malignancies. The MMP inhibitor antibodies, peptides and nucleic acids of the present invention, such as the siRNA that specifically targets MMP14, also can be used to treat or prevent a variety of disorders associated with MMP induced cancer. The antibodies, peptides and nucleic acids are administered to a patient in an amount sufficient to elicit a therapeutic response in the patient (e.g., inhibiting the development, growth or metastasis of cancerous cells; reduction of tumor size and growth rate, prolonged survival rate, reduction in concurrent cancer therapeutics administered to patient). An amount adequate to accomplish this is defined as “therapeutically effective dose or amount.”

The antibodies, peptides and nucleic acids of the invention can be administered directly to a mammalian subject using any route known in the art, including e.g., by injection (e.g., intravenous, intraperitoneal, subcutaneous, intramuscular, or intrademal), inhalation, transdermal application, rectal administration, or oral administration.

In other embodiments, such antibodies that specifically bind or inhibit MMP-3, MMP-9, or MMP-14, may be used therapeutically. Such use of antibodies has been demonstrated by others and may be useful in the present invention to inhibit or downregulate MMPs.

High Throughput Screening for Small Molecules that Modulate MMPs.

In one embodiment, high throughput screening (HTS) methods are used to identify compounds that inhibit MMPs. HTS methods involve providing a combinatorial chemical or peptide library containing a large number of potential therapeutic compounds (i.e., compounds that inhibit MMP14). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS™, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Pharmaceutical Compositions.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Gene Therapy.

In certain embodiments, the nucleic acids encoding inhibitory MMP14 peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory MMP14 peptides and nucleic acids of the present invention, thereby mitigating the effects of over amplification of a candidate gene associated with reduced survival rate.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as decribed in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875). Additional suitable vectors include E1B gene-attenuated replicating adenoviruses described in, e.g., Kim et al., Cancer Gene Ther. 9(9):725-36 (2002) and nonreplicating adenovirus vectors described in e.g., Pascual et al., J. Immunol. 160(9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory MMP14 nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9(8):692-9 (2002).

Combination Therapy.

In some embodiments, the MMP inhibitor are administered in combination with a second therapeutic agent for treating or preventing cancer. In one embodiment, an inhibitory MMP siRNA may be administered in conjunction with a second therapeutic agent for treating or preventing breast, ovarian or colon cancer. For example, an inhibitory MMP siRNA may be administered in conjunction with any of the standard treatments for ovarian cancer including, but not limited to, chemotherapeutic agents including, e.g., alitretinoin, altretamine, anastrozole, azathioprine, bicalutamide, busulfan, capecitabine, carboplatin, cisplatin, cyclophosphamide, cytarabine, doxorubicin, epirubicin, etoposide, exemestane, finasteride, fluorouracil, fulvestrant, gemtuzumab, ozogamicin, hydroxyurea, ibritumomab, idarubicin, ifosfamide, imatinib, letrozole, megestrol acetate, methotrexate, mifepristone, paclitaxel, rituximab, tamoxifen, temozolomide, tretinoin, triptorelin, vincristine, or vinorelbine, and radiation treatment.

The MMP inhibitor and the second therapeutic agent may be administered simultaneously or sequentially. For example, the inhibitory MMP polypeptides and nucleic acids may be administered first, followed by the second therapeutic agent. Alternatively, the second therapeutic agent may be administered first, followed by the inhibitory MMP polypeptides and nucleic acids. In some cases, the inhibitory MMP polypeptides and nucleic acids and the second therapeutic agent are administered in the same formulation. In other cases the inhibitory MMP polypeptides and nucleic acids and the second therapeutic agent are administered in different formulations. When the inhibitory MMP polypeptides and nucleic acids and the second therapeutic agent are administered in different formulations, their administration may be simultaneous or sequential.

In some cases, the inhibitory MMP polypeptides and nucleic acids can be used to target therapeutic agents to cells and tissues expressing MMP and other candidate genes that are related to reduced survival rate.

Administration.

Administration of the MMP inhibitors (e.g., antibodies, peptides and nucleic acids) of the invention can be in any convenient manner, e.g., by injection, intratumoral injection, intravenous and arterial stents (including eluting stents), cather, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., ovarian cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, MMP inhibitors (e.g., antibodies, polypeptides and nucleic acids) of the present invention can be administered at a rate determined by the LD-50 of the polypeptide or nucleic acid, and the side-effects of the antibody, polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the MMP inhibitors (e.g., antibodies, peptides and nucleic acids) of the present invention parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators, including Dharmacon, but typically require complex formulation knowledge. There are a few reports that cite success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

In certain embodiments, the inventors contemplate the use of liposomes, nanocapsules, microparticles, microspheres, lipid particles, vesicles, and the like, for the administration of the MMP14 inhibitory peptides and nucleic acids of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Alternatively, the invention provides for pharmaceutically-acceptable nanocapsule formulations of the compositions of the present invention. Nanocapsules can generally entrap compounds in a stable and reproducible way (Henry-Michelland et al. Attachment of antibiotics to nanoparticles: preparation, drug-release and antimicrobial activity in vitro, Int. J. Pharm. 35, 121-27, 1987; Quintanar-Guerrero et al. Pseudolatex preparation using a novel emulsion-diffusion process involving direct displacement of partially water-miscible solvents by distillation. Int'l J. Pharmaceutics 188(2), 155-64, 1998; Douglas et al. Nanoparticles in drug delivery. Rev. Ther. Drug Carrier Syst. 3, 233-61, 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., Tissue distribution of antitumor drugs associated with polyalkylcyanoacrylate nanoparticles. J. Pharm. Sci. 69, 199, 1980; zur Muhlen et al. Solid lipid nanoparticles (SLN) for controlled drug delivery—Drug release and release mechanism. Euro. J. Pharmaceutics and Biopharmaceutics 45(2), 149-55, 1998; Zambaux et al. Influence of experimental parameters on characteristics of poly(lactic acid) nanoparticles prepared by a double emulsion method. J. Controlled Release 50(1-3), 31-40, 1998; (H. Pinto-Alphandry, A. Andremont and P. Couvreur, Targeted delivery of antibiotics using liposomes and nanoparticles: research and applications. Int. J. Antimicrob. Agents 13, 155-168, 2000); U.S. Pat. No. 5,145,684; and U.S. Pat. No. 6,881,421).

### Kits

The present invention further provides kits for use within any of the above diagnostic methods. Such kits typically comprise two or more components necessary for performing a diagnostic assay. Components may be compounds, reagents, containers and/or equipment. For example, one container within a kit may contain an inhibitory MMP polypeptide and nucleic acid. One or more additional containers may enclose elements, such as reagents or buffers, to be used in the assay. Such kits may also, or alternatively, contain a detection reagent as described above that contains a reporter group suitable for direct or indirect detection of antibody binding.

Kits can also be supplied for therapeutic uses. Thus, the subject composition of the present invention may be provided, usually in a lyophilized form, in a container. The inhibitory MMP14 polypeptides and nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent, e.g., paclitaxel, carboplatin, or other chemotherapeutic agent.

### Example 1

### MMP-14 Role in Branching Morphogenesis

Using a transgenic mouse model, here we found that Mmp14 is highly expressed at the invading edges of TEBs, and that its expression peaks at the height of development of the mammary epithelial tree. We hypothesized that the localization of Mmp14 at the invading front of TEBs may indicate an important role for Mmp14 in the branching process. We postulated further that because of location at the cell surface, Mmp14 may serve also as a bidirectional signal transducer between the invading cell and its surrounding ECM. Testing such a hypothesis in vivo would be complicated by multiple cell types, different ECM molecules and proteases present within the gland that change rapidly and continuously as development progresses. To overcome these obstacles, we used a combination of a transgenic mouse model, primary mammary organoids or a mammary cell line grown in three-dimensional (3D) CL-1 gels (Simian et al., 2001).

Our studies revealed that Mmp14 proteolytic activity was required in dense—but not in sparse—CL-1 gels, surprisingly however, non-catalytic activity of Mmp14 was still required for branching in sparse gels. Because it is known that Itgb1 is necessary for branching in vivo (Taddei et al., 2008), we explored the possibility of an association between Mmp14 and Itgb1. Using immunoprecipitation and FRET analysis, we showed the physical interaction between the two molecules. This finding explained how Mmp14 could activate MAPK signaling, despite the absence of a kinase domain. Furthermore we showed the extracellular domain with or without proteolytic activity as well as the transmembrane/cytoplasmic domain of Mmp14 are required for both branching in CL-1 gels and modulating the level of Itgb1 expression. In summary, our results demonstrate that Mmp14 is a central regulator of invasion and branching and mediates signals from the ECM via cross talk and association with Itgb1.

Cell Culture and Reagents.

Functionally normal mouse mammary epithelial cells, EpH4 (Reichmann et al., 1989), were cultured in 1:1 Dulbecco's Modified Eagle's Medium: Ham's F12 Nutrient Mixture (DMEM/F12), 2% fetal bovine serum, 5 μg/ml insulin, 50 μg/ml gentamycin (Sigma). The following inhibitors were used at the concentrations indicated: PD98059 (40 μM; Calbiochem, San Diego, USA); GM6001 (40 μM; Chemicon/EMD Millipore, Billerica, USA).

Whole Mount β-Gal Staining.

Transgenic mice carrying the LacZ gene under the control of the Mmp14 promoter were used (Yana et al., 2007). Inguinal MGs were isolated from 12 week old wild type (+/+) and Mmp14 (+/−, lacZ) mice. Tissues were collected in ice-cold PBS and fixed for 15 min at room temperature in fix solution (2% formaldehyde, 0.2% glutaraldehyde, 0.02% Nonidet P-40 (NP-40) and 0.01% sodium deoxycholate in PBS). After fixation, tissues were rinsed several times in PBS and stained overnight at 37° C. in the dark with stain solution (5 mM potassium ferricyanide, and 5 mM potassium ferrocyanide in rinse buffer, 1 mg/ml β-Gal, 2 mM MgCl₂, 0.02% NP-40 and 0.01% sodium deoxycholate in PBS). Dehydrated sections of MG tissue were stained with Hematoxylin and Eosin and inspected for β-gal staining.

Branching Morphogenesis Assays.

Branching morphogenesis was induced using slight modifications of previously published protocols (Hirai et al., 1998; Simian et al., 2001). Cell clusters were prepared as follows: EpH4 cells suspended in growth medium containing DNase I were placed on top of agarose-coated wells and incubated at 100 rpm and 37° C. overnight, yielding rounded and well packed clusters. Single cells were removed by centrifugation and the clusters were then washed three times with DMEM/F12. Cell clusters and primary organoids were embedded in collagen-1 gels. Briefly, acid-extracted type I collagen (Koken, Tokyo, Japan) was mixed gently on ice (8 vol) with 1 vol of 10×DMEM/F12, then pH is adjusted to 7.4 with 0.1N NaOH. Collagen solution was added into each well of a 48-well plate, which was then incubated at 37° C. to allow gelation. EpH4 cell clusters or primary organoids were suspended in collagen at 3 mg/ml or 1 mg/ml, poured onto the basal collagen layer and placed at 37° C. for gelation. After gelation of the collagen, growth medium containing 9 nM FGF2 was added to the wells. Branching morphogenesis was assessed using a Nikon Diaphot 300 microscope and clusters were scored as positive when displaying three or more branches of length at least half the diameter of the central cell cluster.

Preparation of Lentivirus.

To transduce FLAG tagged human MMP14F and MMP14F-dCAT (deletion of Tyr112-Pro312) (Itoh et al., 1999; Mori et al., 2002), each cDNA was ligated into pLenti-EF1□-puro, generated in our laboratory. MMP14F-dEC (deletion of Tyr112-Cys516) was made by PCR, and sequence was confirmed by DNA sequencing. Lentivirus plasmids containing shRNA (Mission shRNA; Sigma, St. Louis, USA) against mouse Mmp14 or Itgb1, or Lentivirus plasmids containing MMP14 or mutants were transfected into 293FT cells using FuGene6 (Roche, Basel, Switzerland). Transfected cells were cultured in DMEM containing 5% FBS, 100 U/ml penicillin and 100 μg/ml streptomycin. Culture media was replaced after 24 h with fresh media. 48 h later, recombinant lentivirus was concentrated from filtered culture media (0.45 μm filters) by ultracentrifugation at 25,000 rpm for 90 min (SW41Ti Roter; Beckman Coulter, Brea, USA). To transduce EpH4 cells, 1.0×10⁵ cells were plated in each well of a 6 well-plate, infected with the lentivirus, treated with polybrene for 30 min, and selected by adding 5 μg/ml puromycin to growth medium for 4 days. Lentivirus with scrambled sequence was used as a shRNA control. Target sequences of Mmp14 and Itgb1, and results of 3D CL-1 gel cultures are indicated in Fig. S10. Template plasmids for Ypet and Cypet were purchased from Addgene (http://www.addgene.org) and mutated alanine 206 to lysine as suggested for monomeric form of these fluorophores (Shaner et al., 2005). Monomeric Ypet or Cypet were fused respectively with the c-terminus of ITGB1 or MMP14 by PCR. All the sequences were confirmed by sequencing.

Quantitative RT-PCR Analysis.

Total RNA was isolated using the QIAGEN RNeasy Mini kit (Valencia, USA). 100 ng of total RNA was used to synthesize cDNA using SuperScript II First-Strand Synthesis System (Invitrogen, Carlsbad, USA). Mmp14 was amplified with 5′-GAGATCAAGGCCAATGTTCG (SEQ ID NO: 10) and 5′-GTCCAGGGCTCGGCAGAATC (SEQ ID NO: 11) primers or with 5′-CATCTTCTTGGTGGCTGTG (SEQ ID NO: 12) and 5′-TGACCCTGACTTGCTTCC (SEQ ID NO: 13) primers. Itgb1 was amplified with 5′-GGAGATGGGAAACTTGGTGG (SEQ ID NO: 14) and 5′-CCCATTCACCCCATTCTTGC (SEQ ID NO: 15) primers. As a control for total RNA, RT-PCR for 18S rRNA was performed with 5′-TCGGAACTGAGGCCATGATT (SEQ ID NO: 16) and 5′-CCTCCGACTTTCGTTCTTGATT (SEQ ID NO: 17) primers. Real-time PCR was performed using the LightCycler System and Fast Start DNA Master SYBR Green I (Roche) following manufacturer's instructions.

Western Blotting.

Samples were lysed using modified RIPA buffer (50 mM HEPES pH 7.4, 150 mM NaCl, 10% glycerol, 1% Triton X-100, 10 mM sodium pyrophosphate containing 1.5 mM MgCl₂, 1 mM EGTA, 1% sodium deoxycholate, 0.25 mM Na₃VO₄, 100 mM NaF and proteinase inhibitor cocktail (EMD Millipore, Billerica, USA)). Protein concentration was determined using the BCA Protein Assay kit (Thermo Scientific, Waltham, USA), following the manufacturer's instructions. Protein samples (10 μg) were mixed with Laemmeli sample buffer and heated at 95° C. for 5 minutes. Samples were loaded into a pre-cast 4-20% tris-glycine polyacrylamide gel (Invitrogen) using the NOVEX system (Invitrogen). Resolved proteins were transferred to nitrocellulose membrane (Whatman, Maidstone, UK) followed by blocking in PBS, 0.05% Tween-20 with 5% w/v non-fat dry milk for 1 hour at room temperature (RT). Membranes were incubated overnight at 4° C. in 5% BSA, 0.1% Tween-20 in PBS containing antibodies that recognize either phosphorylated Erk1/2, total Erk1/2 (Cell Signaling). Anti-Mmp14 (Abcam, Cambridge, USA), anti-Itgb1 (Santa Cruz Biotechnology, Santa Cruz, USA), anti-Itgb1 p788/789 (Abcam), anti-LAM A/C (Santa Cruz Biotechnology), anti-Actin (Abcam), anti-FLAG (Sigma) and rabbit IgG (Sigma) antibodies were used for immunoblotting. Primary antibodies were detected with HRP conjugated anti-IgG (Thermo) and the Pierce SuperSignal detection kit. Chemiluminescence ignal was captured with a FluorChem 8900 analysis system (Alpha Innotech, San Leandro, USA).

Immunoprecipitation.

Samples were lysed at room temperature (RT) with 50 mM HEPES pH 7.4, 150 mM NaCl, 1% Brij98 (Sigma), 1.5 mM MgCl₂, phosphatase inhibitor cocktail (Sigma) and proteinase inhibitor cocktail (EMD Millipore). Lysate containing 5 mg protein was incubated with □□μg of control rabbit-IgG or anti-Mmp14 for 16 hours at RT. Precipitation was performed with protein-G sepharose (GE Healthcare, Little Chalfont, UK). 10% of total precipitates were loaded to western blotting. For detecting Itgb1, anti-Itgb1 (Santa Cruz Bioteech.) was used.

Gel Stiffness Probed by AFM.

Gels were prepared by adding 100 μl of collagen solutions (1 or 3 mg/ml) to the glass surface of a 35-mm culture dish with a 14-mm diameter bottom-glass coverslip (MatTek, Ashland, USA) and incubating the samples for 20 min at 37° C. to allow gelation. Gel stiffness was characterized by measuring the Young's elastic modulus (E) using an atomic force microscope (AFM) (Bioscope; Bruker AXS, Santa Barbara, USA) as previously described (Alcaraz et al., 2003; Alcaraz et al., 2011). Briefly, three force-indentation (F-□) curves were acquired in at least nine gel locations for each independent experiment (n≧2). A contact elastic model was fitted to the loading part of each F-□ curve to obtain E.

### Results

Mmp14 Expression Peaks During Puberty, and is Highly Elevated at the Invading Front of Mammary Gland End Buds.

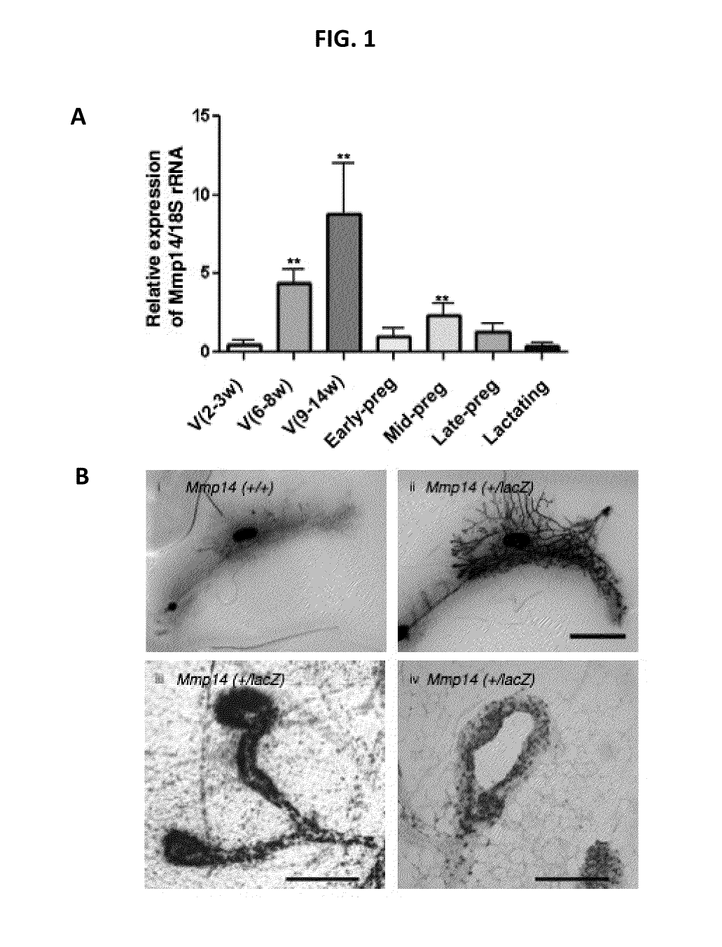

We measured the levels of Mmp14 expression in different stages of MG development in virgin, pregnant and lactating mice using quantitative RT-PCR. Mmp14 expression increased with development of the mammary epithelial tree during branching morphogenesis in virgin mice (FIG. 1a). However, Mmp14 expression plunged at the onset of pregnancy before mildly increasing during mid-pregnancy (coinciding with the stage of pregnancy that alveoli form) and subsiding again in late pregnancy and during lactation (FIG. 1a). □-galactosidase staining of whole mounted MG isolated from 5-week-old mice carrying a LacZ reporter downstream of the endogenous Mmp14 promoter (Yana et al., 2007) revealed expression of Mmp14 in epithelial cells, especially at the tips of ducts and/or TEBs (FIG. 1b-i-iii). Analysis of tissue sections indicated that the Mmp14 promoter was active prominently also in myoepithelial cells (FIG. 1b-iv), suggesting that Mmp14 functions at the interface of the invading epithelium and ECM.

Mmp14 Catalytic Activity is Required for Invasion/Branching Only in Dense—but not Sparse—Collagen Gels.

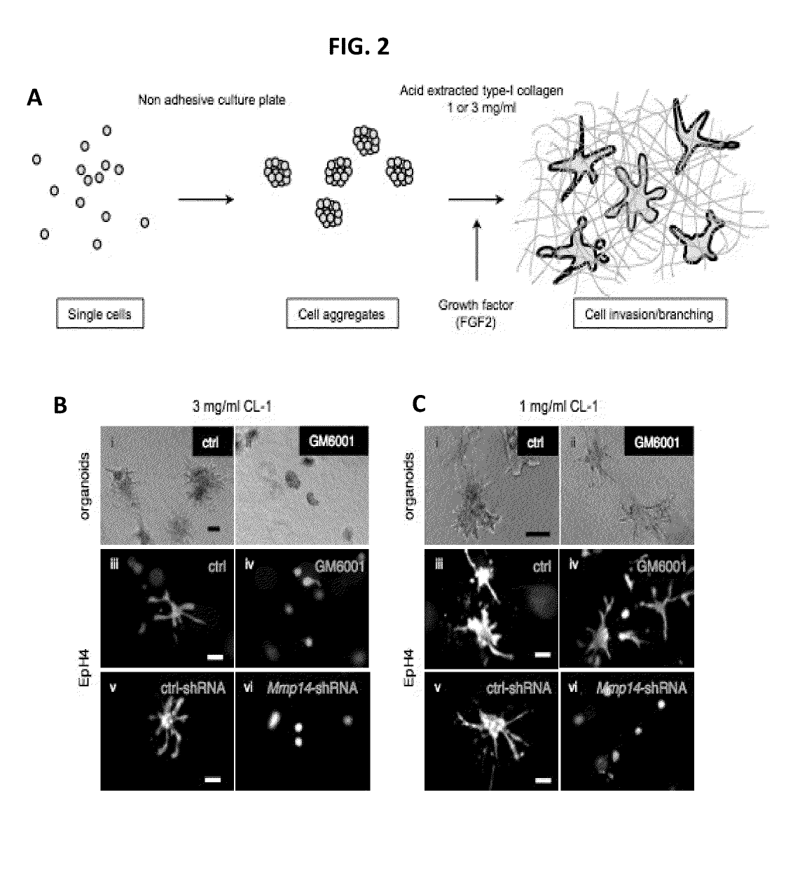

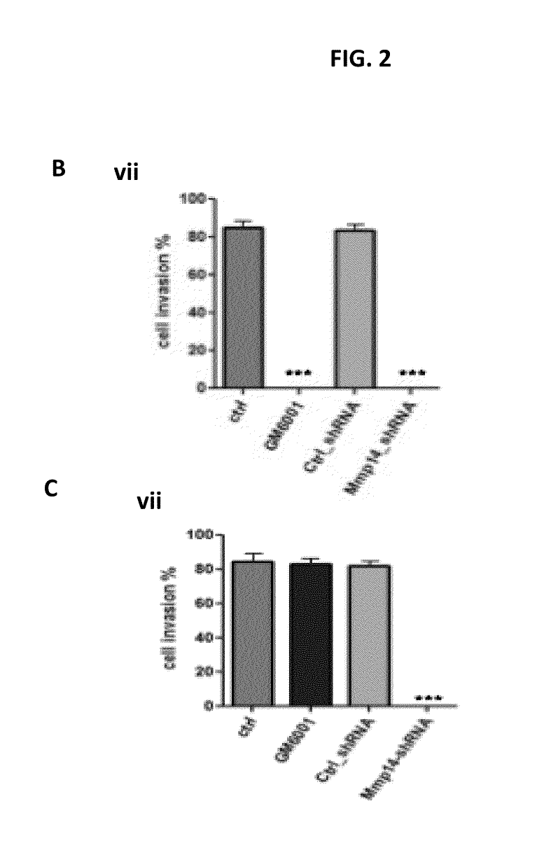

To dissect the role of Mmp14 in mammary invasion/branching, we utilized two culture models that simulate mammary epithelial branching: primary mammary organoids (Fata et al., 2007; Simian et al., 2001) and aggregated cellular clusters (Hirai et al., 1998) of a functionally normal mouse mammary epithelial cell line (EpH4) (Reichmann et al., 1989), in each case embedded within a CL-1 gel (FIG. 2a). The physiological relevance of this model is illustrated by the rich presence of CL-1 in the stroma surrounding epithelial ducts in the murine MG (Fig. S1) (Williams and Daniel, 1983).

To mimic the CL-1-rich ECM found in the MG, we used two different concentrations of CL-1: 1 mg/ml (sparse) and 3 mg/ml (dense). The denser concentration is actually representative of the pre-malignant MG (Levental et al., 2009), but has been used commonly nonetheless by us and others to model branching of the mammary epithelium (Brinkmann et al., 1995; Hirai et al., 1998; Janda et al., 2002; Mori et al., 2009; Simian et al., 2001). Upon addition of fibroblast growth factor-2 (FGF2), both primary organoids and EpH4 cells invaded dense CL-1 gels (FIG. 2b-i,iii). Invasion was completely abrogated by addition of either the broad-spectrum MMP inhibitor GM6001 (FIG. 2b-ii, iv) or tissue inhibitor of metalloproteinases (TIMP)-2 (Simian et al. 2001, not shown) in these gels, which have an average pore size that is smaller than the size of an average cell (Alcaraz et al., 2011). Silencing Mmp14 with shRNA decreased Mmp14 expression by ˜90% (Fig. S2) and also completely inhibited MEC invasion in CL-1 under these conditions (FIG. 2b-v-vii).

Using atomic force microscopy, we found that sparse 1 mg/ml CL-1 gels better approximate the mechanical microenvironment of the murine mammary gland (Fig. S3) (Levental et al., 2009; Paszek et al., 2005). Under these conditions, primary organoids and MECs still invaded in response to FGF2 (FIG. 2c-i,iii), regardless of whether GM6001 (FIG. 2c-ii, iv) or TIMP2 (data not shown) were added or not. Unexpectedly however, expression of Mmp14 itself was still required: shRNA knockdown of Mmp14 blocked MEC invasion completely (FIG. 2c-v-vii). These data suggest that Mmp14 has multiple activities involved in the regulation of invasion/branching morphogenesis in the MG, at least one of which is non-proteolytic in nature. Therefore, we concentrated on elucidating the mechanism by which the non-catalytic activity of Mmp14 is involved in branching morphogenesis in physiologic CL-1 gels.

MAPK Activity and Cross Signaling Between Mmp14, Erk and Itgb1 are Involved in Branching Morphogenesis.

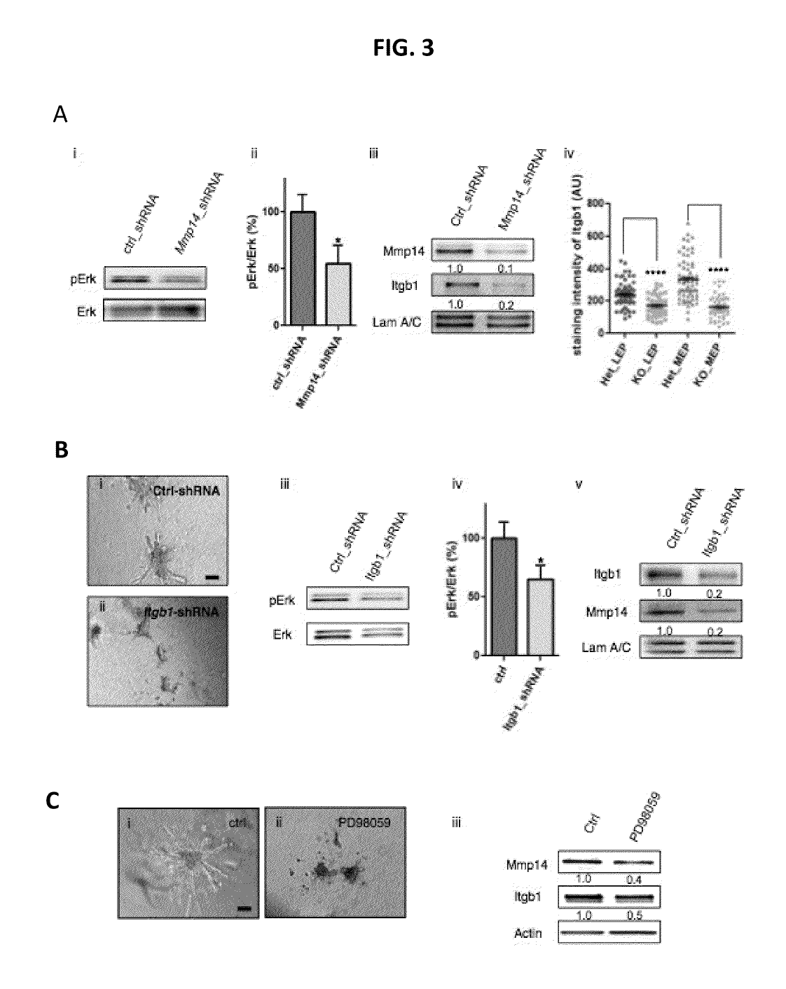

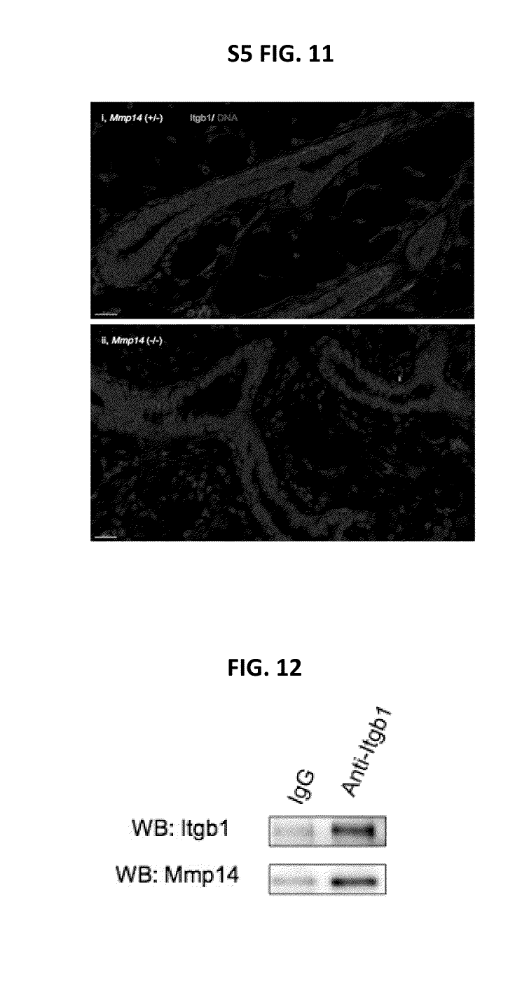

We had previously shown the necessity of MAPK signaling for alveologenesis by primary MECs within 3D laminin-rich ECMs (Fata et al., 2007). We asked whether or not the non-catalytic mechanism through which Mmp14 orchestrates branching in physiologic CL-1 involves MAPK activity. Silencing Mmp14 with shRNA reduced MAPK activation in half (FIG. 3ai, ii). However, MAPK activity did not change in response to addition of GM6001 (FIG. 10), suggesting that MAPK activation does not depend on the proteolytic activity of MMPs and that a non-proteolytic function of Mmp14 is responsible for MAPK activity. However, our analysis of 20 amino acids sequence of Mmp14 cytoplasmic tail (Sato et al., 1994) did not reveal any homology to known kinase domains. How might then Mmp14 affect MAPK activity? One possibility is that Mmp14 regulates downstream signaling through a partner molecule with kinase activity. A search of molecules that were altered when Mmp14 was silenced by shRNA showed Itgb1 was dramatically reduced (FIG. 3aiii). This was not the result of an off-target effect of Mmp14 shRNA: we observed this with multiple shRNA, and examination of MGs from Mmp14 knockout mice (FIG. 11) also demonstrated a significant reduction in Itgb1 expression in both luminal and myoepithelial cells (FIG. 3aiv). A small shRNA screen of collagen receptors with known kinase activity (Itgb1, Discoidin domain receptors: Ddr-1 and -2) confirmed that silencing Itgb1 also blocked branching of EpH4 cells in 1 mg/ml CL-1 gels (FIG. 3bi, ii). Silencing Ddr1 or Ddr2 did not block branching in the same conditions (data not shown). Itgb1 silenced cells showed significantly reduced MAPK activation (FIG. 3biii,iv). Significantly, silencing Itgb1 resulted in a dramatic decrease in Mmp14 levels (FIG. 3bv). These results indicated that Mmp14 and Itgb1 modulate each other's expression in addition to modulating MAPK activity. Preventing MAPK activation by a small molecule MEK inhibitor (PD95089) reduced invasion/branching (FIG. 3ci, ii) and also reduced expression of Mmp14 and Itgb1 (FIG. 3ciii), Confirming the Three-Way Connection. Src Inhibition Showed Similar Inhibition of the levels of Mmp14 and Itgb1 (data not shown), indicating the involvement of downstream intermediates. In sum, these data suggest that Mmp14, Itgb1 and MAPK activation are all connected reciprocally during branching morphogenesis, and specifically that Mmp14 cooperates in a proteolytic ally-independent manner with Itgb1 to activate Erk and facilitate branching in sparse CL-1 gels.

Co-Immunoprecipitation and FRET Reveal a Direct Association Between Mmp14 and Itgb1.

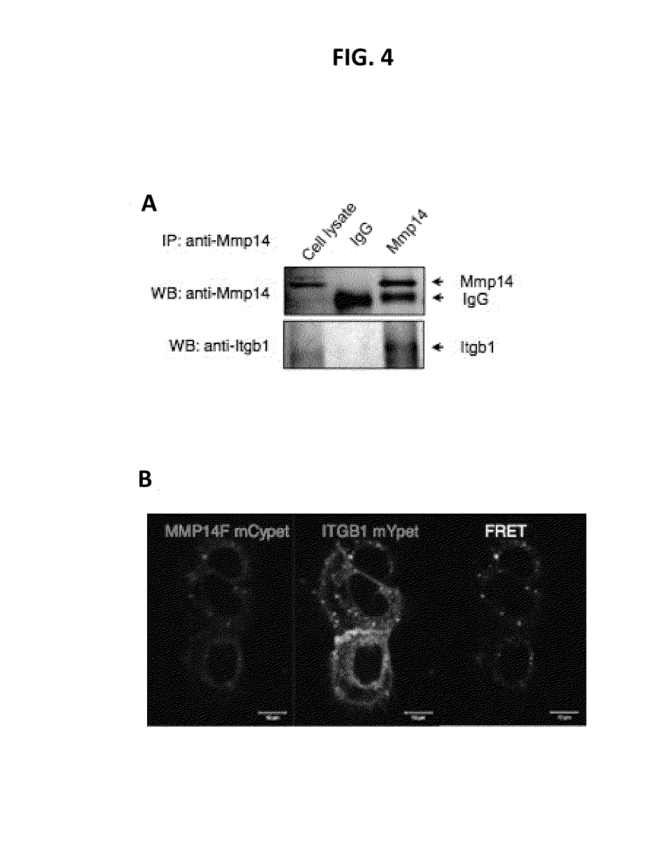

To explore the nature of the Itgb1 and Mmp14 partnership that activates MAPK, we immunoprecipitated endogenous Mmp14 from 3D cultures of branching EpH4 cells and immunoblotted for Itgb1 in the precipitated fraction. Itgb1 was present in this fraction (FIG. 4a). Reverse immunoprecipitation was also performed to confirm this association (FIG. 12). Since a demonstration of co-precipitation alone is not sufficient to show direct interactions, we sought additional evidence for protein-protein association. We performed FRET analysis (Nguyen and Daugherty, 2005; Shaner et al., 2005) using monomeric (m) Cypet-tagged MMP14 and mYpet-tagged ITGB1. Excitation of mCypet-MMP14 elicited fluorescence of mYpet-ITGB1 in a substantial portion of EpH4 cells, supporting a direct association (within ˜10-100 Å, (Forster, 2012)) between these two molecules (FIG. 4b).

Assigning Functional Activities to the Non-Catalytic Domains of MMP14.

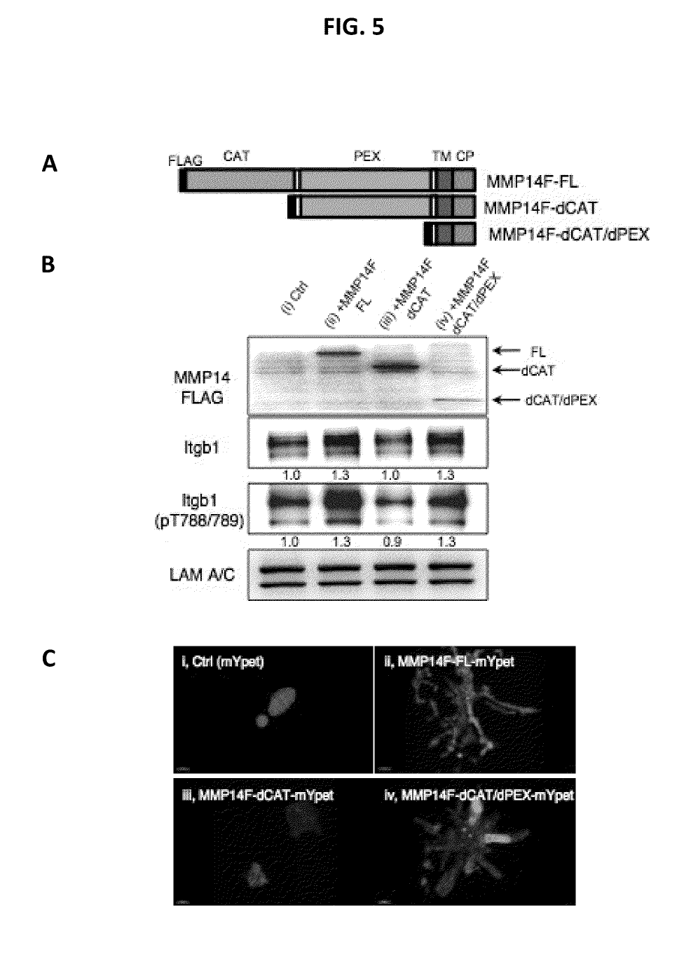

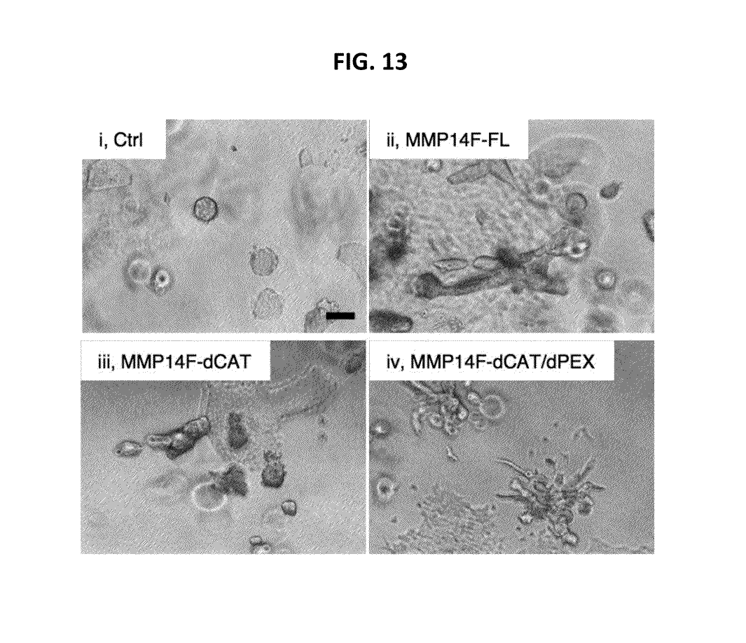

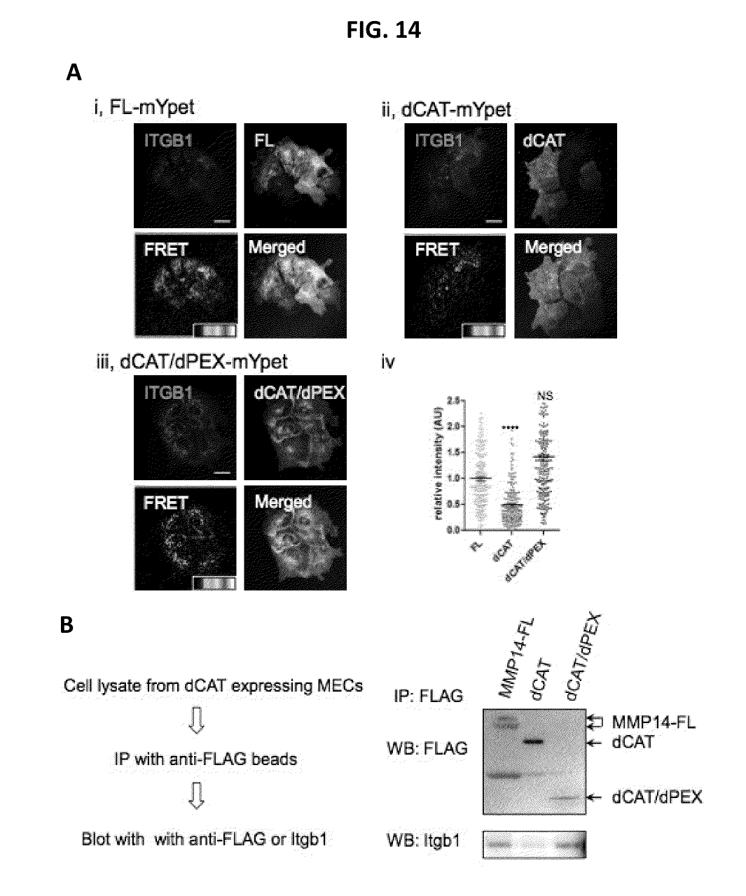

To dissect the non-catalytic activities of Mmp14 for functional relevance, we engineered two constructs: catalytic domain-deleted mutant (MMP14F-dCAT; FIG. 5a) and catalytic/hemopexin domains-deleted mutant (MMP14-dCAT/dPEX; FIG. 5a). To prevent interference from endogenous Mmp14, we silenced the endogenous enzyme with shRNA, and then introduced the exogenous FLAG tagged full length MMP14 (MMP14F-FL; FIG. 5a), MMP14F-dCAT, MMP14F-dCAT/dPEX and the vector control using lentivirus. Over expression of MMP14F-FL restored the level of Itgb1 as expected (FIG. 3 and FIG. 5b). However, whereas MMP14F-dCAT/dPEX also restored the Itgb1 level and its activity (Itgb1 pT788/789; FIG. 5b), MMP14F-dCAT did not (FIG. 5b; the ratios of Itgb1 level/loading control (LAM A/C) are shown below each lane). To establish that the biochemical analysis is relevant to morphogenetic behavior, and to observe the behavior of mutants more directly, we tagged all the constructs at the C-terminus with mYpet, and confirmed that the mYpet tag did not interfere with the functional behavior (FIG. 13). We tested parallel transduced-cultures in the branching assays, and found that MMP14F-FL and MMP14F-dCAT/dPEX restored the invasion in cells that were silenced for endogenous Mmp14, but not in cells expressing MMP14F-dCAT (FIG. 5c). To confirm the association between each mutant and Itgb1, FRET analyses were performed between mCypet tagged ITGB1 and mYpet tagged MMP14-FL, -dCAT and -dCAT/dPEX (FIG. 14). MMP14-FL and dCAT/dPEXs showed significantly higher FRET signals compared to MMP14-dCAT, further supporting the results shown in FIG. 5. These findings indicate that the transmembrane/cytoplasmic domain of MMP14 is required for regulation of Itgb1 levels and activity as well as the cell's ability to invade and branch in CL-1, and that the catalytic activity and the hemopexin domain are not necessary for these functions. It is surprising that the construct, MMP14F-dCAT, does not allow either of the two functions described above despite the fact that the transmembrane/cytoplasmic domain is still present. This finding raises the possibility that the hemopexin domain exerts inhibitory function on the transmembrane/cytoplasmic domain only if the catalytic domain is not present. The action of MMP14F-dCAT was confirmed further by re-expressing MMP14 catalytic inactive mutant in Mmp14-silenced EpH4 cells, and it restored branching/invasion in sparse gels (data not shown). It is likely that the protein folding and/or localization of MMP14 are altered without the catalytic domain. This mechanism is under investigation in our laboratory.

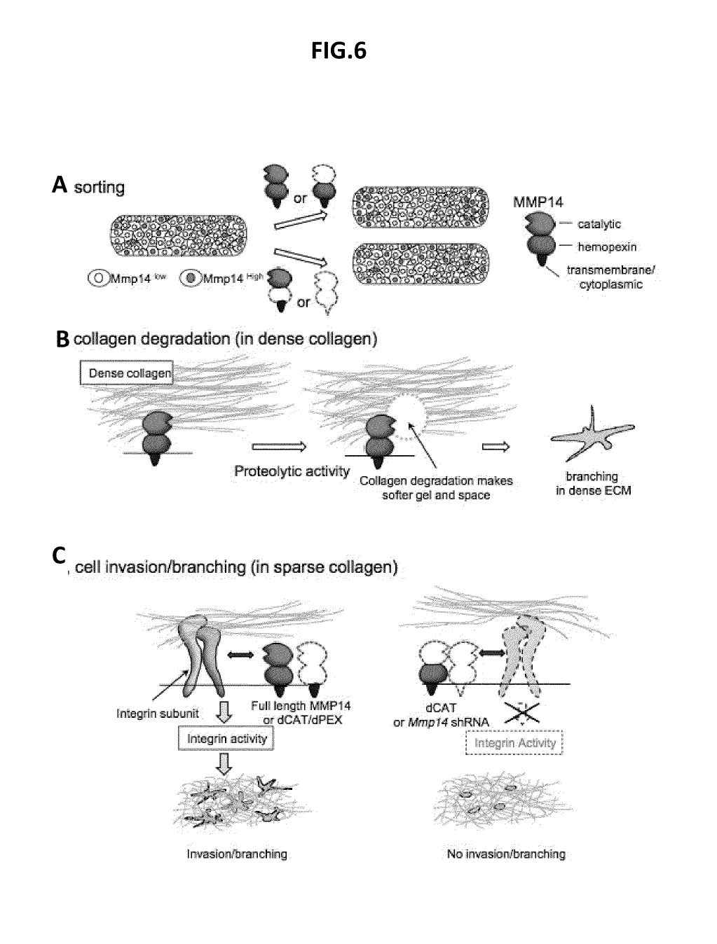

We showed previously that mammary epithelial cells utilize the hemopexin domain of Mmp14 to sort cells to the branching/initiating front (Mori et al., 2009). We also have shown recently that the proteolytic activity of Mmp14 is needed to degrade dense collagen gels to generate a path for branching (Alcaraz et al., 2011). Here, we demonstrate that it is the transmembrane/cytoplasmic domain that is needed for signaling to MAPK via its interaction with Itgb1 (FIG. 6c). Thus there is division of labor between the different domains of MMP14: depending on the context—the density of the ECM in this case—the domains perform different tasks to complete branching morphogenesis in the mammary gland.

### Discussion

During branching morphogenesis, epithelia form tubular/branching structures through well-controlled processes of cellular invasion and proliferation. MMPs are key metalloproteinases that degrade ECM, and are involved in both mammary branching morphogenesis (Fata et al., 2004; Lu and Werb, 2008; Simian et al., 2001; Sympson et al., 1994) and cancer cell invasion (Brinckerhoff and Matrisian, 2002). Mmp14 is a membrane tethered MMP (Sato et al., 1994) and is characterized as a collagenase (Alcaraz et al., 2011; Ohuchi et al., 1997). It is also a known activator of other MMPs (Knauper et al., 1996; Sato et al., 1994) and a sheddase for surface molecules (Endo et al., 2003; Kajita et al., 2001). The enzyme plays a role in morphogenesis of ureteric buds (Meyer et al., 2004) where Mmp14KO mice show a substantial decrease in ureteric branching (Riggins et al., 2010); it is also instrumental in lung alveologenesis (Atkinson et al., 2005; Kheradmand et al., 2002). However in these and other reports on the role of Mmp14 in morphogenesis, the emphasis has been essentially on its proteolytic/catalytic activity, and very little attention has been paid to the non-catalytic domains of this important molecule.

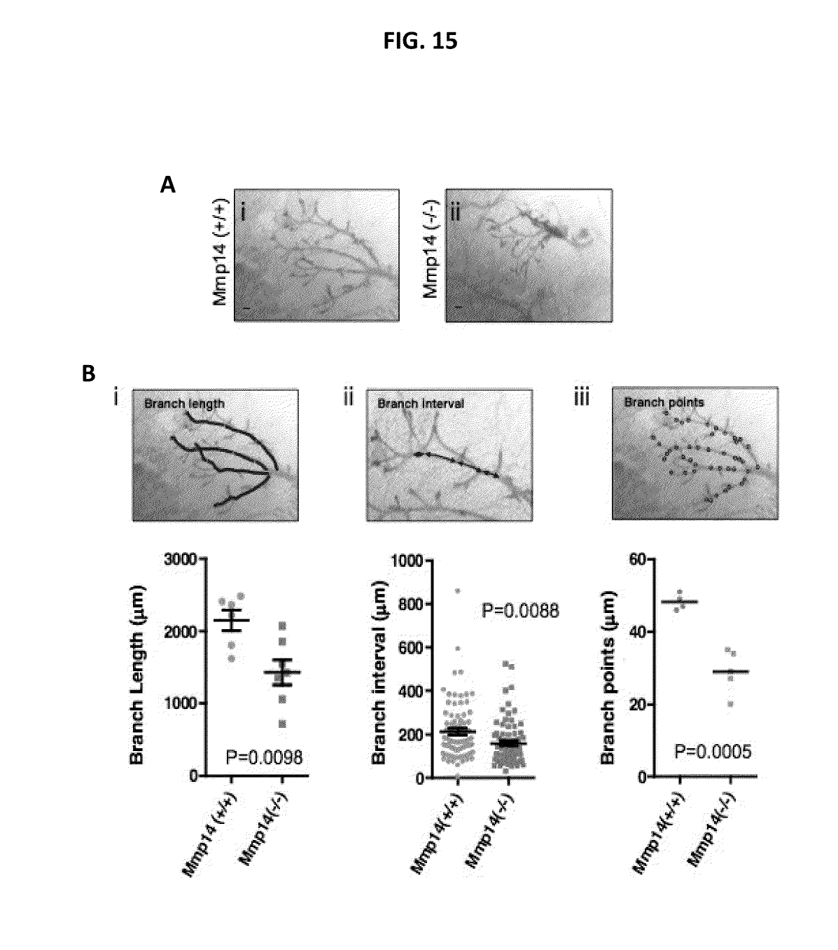

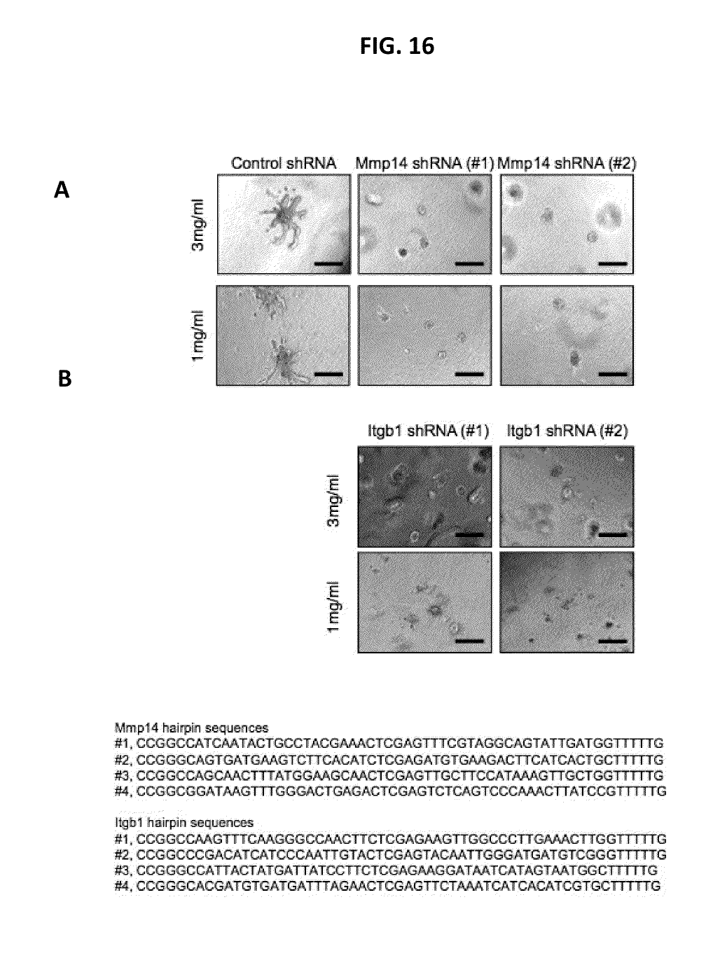

Mmp14 is expressed in normal mammary glands during ductal branching (Szabova et al., 2005; Wiseman et al., 2003). It is highly upregulated in β1,4-galactosyltransferase-1 knockout mice, which display enhanced mammary glands side-branching (Steffgen et al., 2002). Our data presented here also show that Mmp14 is highly expressed in the mammary glands of virgin mice during branching morphogenesis. However, whereas Mmp14 expression was reported mainly in the stroma of mammary glands of FVB mice (Szabova et al., 2005; Wiseman et al., 2003), our analysis using Mmp14 (+/lacZ, heterozygote, C57BL/6) revealed high promoter activity essentially in mammary epithelial cells, especially in the end buds (FIG. 1b). We have confirmed that the expression of Mmp14 tracks with the promoter activity in our study (data not shown). It is possible that the differences in Mmp14 localization in our study and those mentioned above are due to differences in the mouse background. However, in FVB back-crosses, we have confirmed the expression of Mmp14 also in the epithelia. Therefore we believe that the epithelial Mmp14 plays a direct role in controlling growth and extension of epithelial branching. In fact, the mammary glands from back-crossed Mmp14 KO (C57BL/6) mouse showed reduced ductal elongation, branching intervals and branching points compared to the wild type (FIG. 15).

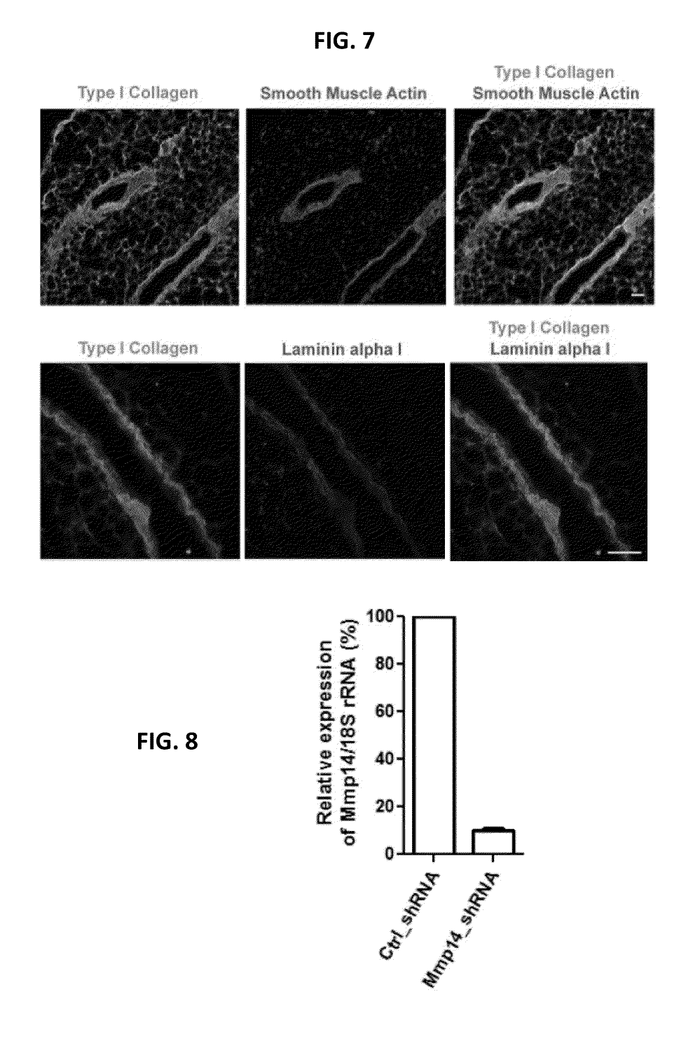

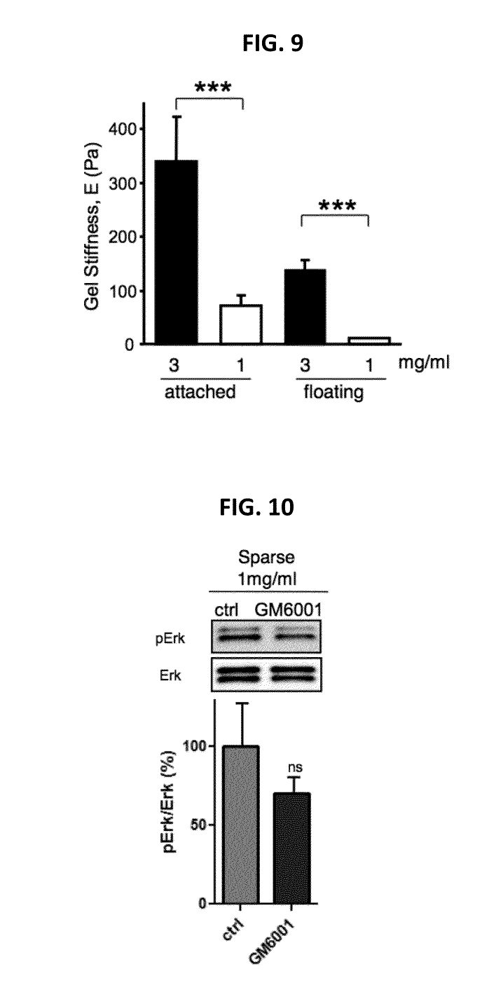

The complex surges in growth factors and hormones during mammary gland development in vivo make it difficult to understand the molecular mechanisms by which Mmp14 contributes to specific stages of branching morphogenesis. We thus chose 3D culture models of collagen-1 (CL-1) gels using both mammary organoids or mammary epithelial cells (MECs) previously utilized by us and others (Brinkmann et al., 1995; Hirai et al., 1998; Provenzano et al., 2009; Simian et al., 2001). Here we used different concentration of native acid-extracted CL-1 to mimic the conditions similar to those found in vivo. We demonstrate that whereas mammary epithelial cells use the proteolytic activity of Mmp14 for invasion/branching in dense (3 mg/ml) CL-1, this activity is dispensable in sparse (1 mg/ml) CL-1 gels. However, intriguingly, we found that the Mmp14 molecule itself is still necessary for mammary epithelial cells to branch, indicating that Mmp14 possesses previously unknown non-proteolytic functions in development. Mammary epithelial ducts are surrounded by basement membrane components such as laminins/type-IV or type-I collagen (FIG. 7 and data not shown for type IV collagen). The proteolytic functions of Mmp14 may be direct or indirect (e.g. induction of Mmp2) and is undoubtedly used for degrading and remodeling these basement membrane components during branching morphogenesis.

To demonstrate the non-proteolytic function of Mmp14, a physiologically-relevant assay was needed. In addition to using branching as an end point, we wanted to know the pathways by which non-catalytic domains would signal for branching. A number of intracellular signaling cascades are essential transducers of microenvironmental stimuli in MECs and are known to mediate morphogenesis. For example, c-Src (−/−) mice have defects in mammary ductal elongation (Kim et al., 2005), and in MEK-inhibited mammary epithelial organoids in 3D laminin-rich gels there are defects in alveologenesis (Fata et al., 2007). We thus considered using MAPK activation as an additional end point in sparse CL-1 gels where proteolytic activity of Mmp14 was not necessary. However, our analysis of the Mmp14 short cytoplasmic domain indicated it did not contain any known kinase domain, suggesting that Mmp14 may have to couple to a partner with such activity. Since our assay was invasion and branching in collagen gels, we suspected involvement of a collagen receptor. A selective silencing of different collagen receptors revealed that silencing Itgb1 exhibited the same phenotype as silencing Mmp14. We demonstrate that Mmp14 indeed associates directly with Itgb1 (FIG. 4) indicating a possible link between Mmp14 and Itgb1 in activation of MAPK signaling. Surprisingly, silencing Mmp14 affected the level of Itgb1 in MECs. This finding has a physiological counterpart in vivo: analysis of mammary gland tissue showed that the level of Itgb1 was dramatically lower in Mmp14 knockout mice than in the heterozygotes (FIG. 3a-iv and FIG. 11). In a 3D model of human breast cancer cells (Petersen et al., 1992), we had shown previously that ITGB1 and MAPK modulate each other's levels reciprocally, and are involved in regulating 3D architecture in laminin-rich gels (Wang et al., 1998). Our results here indicate that Mmp14 is involved in the reciprocal association between Itgb1 and the activation of MAP kinase in mammary epithelial cells in CL-1 gels, and that this association is required for branching morphogenesis. Our preliminary experiments with integrin β3 (Itgb3) indicated that this integrin is also involved in the reciprocal association between Mmp14 and Itgb1 in MECs (data not shown), suggesting that the protein complex of Mmp14/Itgb1 might be only a piece of a larger complex of proteins that includes also other integrins and possibly growth factor receptors. We will be addressing the other molecular partners of Mmp14 in future experiments.

Having demonstrated how Mmp14 signals even in the absence of its proteolytic activity during branching morphogenesis, we examined which domain is required for signaling to Itgb1. To answer this question unambiguously, we silenced endogenous Mmp14 in MECs and then re-expressed FLAG tagged full length MMP14 or its deletion mutants. Surprisingly, whereas the full length MMP14 and the extracellular domain deleted mutant restored the level of Itgb1 and branching, the catalytic domain-deleted mutant did not. These results indicated that the transmembrane/cytoplasmic (TM/CP) domain of MMP14 has a key function in signaling, but that the hemopexin domain has an inhibitory effect, but only when its catalytic domain is deleted. Previous studies had demonstrated a naturally-derived cleaved form of catalytic domain of MMP14 can act as a dominant negative regulator of wild type MMP14 proteolytic activity (Itoh et al., 2001; Lehti et al., 2002). Here we demonstrate that when the catalytic domain is absent the hemopexin domain has an inhibitory effect on both branching and activation of Itgb1 in sparse CL-1 gels. The catalytic domain-deleted mutant may have differences in protein structure or binding partners that cause an inhibitory activity on the association with Itgb1 and branching. Combining the present findings with the previous reports suggests that cells use the different domains of Mmp14 not only for controlling the proteolytic actions of Mmp14, but also for regulating integrin function, and signaling in a collagenous microenvironment.

We demonstrated previously that the interaction between Mmp14 hemopexin domain and CD44 determined the motility of mammary epithelial cells resulting in the sorting of cells to the branching initiation points (Mori et al., 2009). We have demonstrated also that the proteolytic activity of Mmp14 is required for branching in dense ECM (Alcaraz et al., 2011). These reports suggest that mammary epithelial tissue recruits the Mmp14-expressing cells to the tip of the branching bud to degrade the local collagen in order to clear a path for penetration. We show here that once this is accomplished, the catalytic activity is dispensable. However, the Mmp14 molecule is still required for branching, and the TM/CP domain of MMP14 is the minimum required domain for controlling both the level of Itgb1 and branching in a sparse collagen microenvironment. Whereas during cell sorting the hemopexin domain is used for associating with CD44 (Mori et al., 2009), this domain can exert an inhibitory activity on Itgb1 levels leading the decreased signaling and branching in endogenously-silenced Mmp14 mammary epithelial cells, as shown in this study (Scheme in FIG. 6c).

In conclusion, we posit that the Mmp14-dependent Itgb1 regulation, in combination with increased expression of Mmp14 observed at the tips of invading mammary end buds, may constitute a signaling module and dynamics relevant to the invasive fronts of branching tissues. We show that under specific conditions, non-catalytic domains of MMP14 play crucial roles for cells to invade into stroma during development. Because these same developmental programs are subverted in malignant cells during tumor progression, our results may shed light on why MMP inhibitors, which only targeted the catalytic domains failed so dramatically in clinical trials (Overall and Kleifeld, 2006a; Overall and Kleifeld, 2006b). We also suggest that domains of Mmp14 other than its catalytic domain could be targets for controlling cellular invasion in cancer.

### Example 2

### The Hemopexin Domain of MMP3 is Responsible for Mammary Epithelial Invasion and Morphogenesis Through Extracellular Interaction with HSP90b

Matrix metalloproteinases (MMPs) are crucial mediators in sculpting tissue architecture and are required for many physiological and pathological processes. MMP3 has been shown to regulate branching morphogenesis in the mammary gland. Ectopic expression of proteolytically active MMP3 in mouse mammary epithelia triggers supernumerary lateral branching and, eventually, tumors. Using a three-dimensional collagen-I (Col-1) gel assay that simulates epithelial invasion and branching, we show that it is the hemopexin domain that directs these processes. Using three different engineered constructs containing a variation on MMP3 structural domains, we confirmed the importance of the hemopexin domain also in primary organoids of the mammary gland. A proteomic screen of MMP3-binding partners surprisingly revealed that the intracellular chaperone heat-shock protein 90 b (HSP90b) is present extracellularly, and its interaction with the hemopexin domain of MMP3 is critical for invasion. Blocking of HSP90b with inhibitory antibodies added to the medium abolished invasion and branching. These findings shift the focus from the proteolytic activity of MMP3 as the central player to its hemopexin domain and add a new dimension to HSP90b's functions by revealing a hitherto undescribed mechanism of MMP3 regulation. Our data also may shed light on the failure of strategies to use MMP inhibitors in cancer treatment and other related disorders.

Prior to the defining functions of the mammary gland (i.e., pregnancy and lactation), the female mammal develops an epithelial tree through branching morphogenesis. During this process, epithelial cells have to mobilize the necessary machinery for invasion of the growing ducts into the fat pad and the formation of secondary and tertiary branches to complete the eventual adult mammary architecture. It has been shown that the success of this process relies on the activities of a number of matrix metalloproteinases (MMPs) (Fata et al. 2004; Khokha and Werb 2011). Paradoxically, the loss of mammary structure also is dependent on MMPs. Indeed, we showed two decades ago that during the process of involution, up-regulation of MMP3 is responsible for the collapse and remodeling of the alveoli of lactating mice, indicating the intimate connection between functional differentiation and tissue structure (Talhouk et al. 1991, 1992). Conditional activation of MMP3 in functionally normal mouse mammary epithelial cells led to cleavage of E-cadherin and epithelial-to-mesenchymal transitions (EMT) (Lochter et al. 1997a). We also showed that ectopic expression of constitutively active MMP3 in mammary epithelia enhanced lateral branching and induced precocious alveolar development in virgin mice (Sympson et al. 1994). As these animals aged, the stroma was profoundly altered in both structure and function (Thomasset et al. 1998), and mice eventually developed mammary tumors that exhibited chromosomal aberrations (Sternlicht et al. 1999). The mechanism involved a change in the cytoskeleton and cell shape through induction of RAC1B, a spliced isoform of RAC1 found in human breast tumors (Schnelzer et al. 2000). Addition of MMP3 or the expression of RAC1B also led to formation of reactive oxygen species (ROS) and genomic instability (Radisky et al. 2005).

Because the proteolytic activity of MMPs resides within the catalytic domain, it has been generally assumed that this domain is responsible for all of the functions of MMPs. More recently, some biochemical literature has indicated that the noncatalytic domains of certain MMPs, such as MMP9, MMP12, and MMP14, may also have activities in mammalian cell lines (Mori et al. 2002; Wang et al. 2004; Dufour et al. 2008; Sakamoto and Seiki 2009). The failure of clinical trials based on inhibitors of MMP catalytic domains (Overall and Kleifeld 2006) suggested to us that the other domains of MMP3 may have functions in invasion and possibly cancer.

Here we show that overexpression of MMP3 constructs without catalytic activity is sufficient to direct mammary epithelial invasion in collagen-I (Col-1) gels. Additionally, the functional activity requires the surprising interaction of heat-shock protein 90β (HSP90β) with MMP3 in the extracellular milieu. This interaction is necessary for invasion and branching in not only cultured cells, but also primary organoids where the mammary architecture remains intact. We believe that these findings introduce an alternative to the classic paradigm of MMP3 activity and point to an HSP90β-mediated regulation of MMP3 function essential for epithelial invasion and mammary morphogenesis.

### Results: the Hemopexin Domain of MMP3 is Required for a Change in Cell Shape in Two-Dimensional (2D) Substrata and Invasion in Boyden Chambers

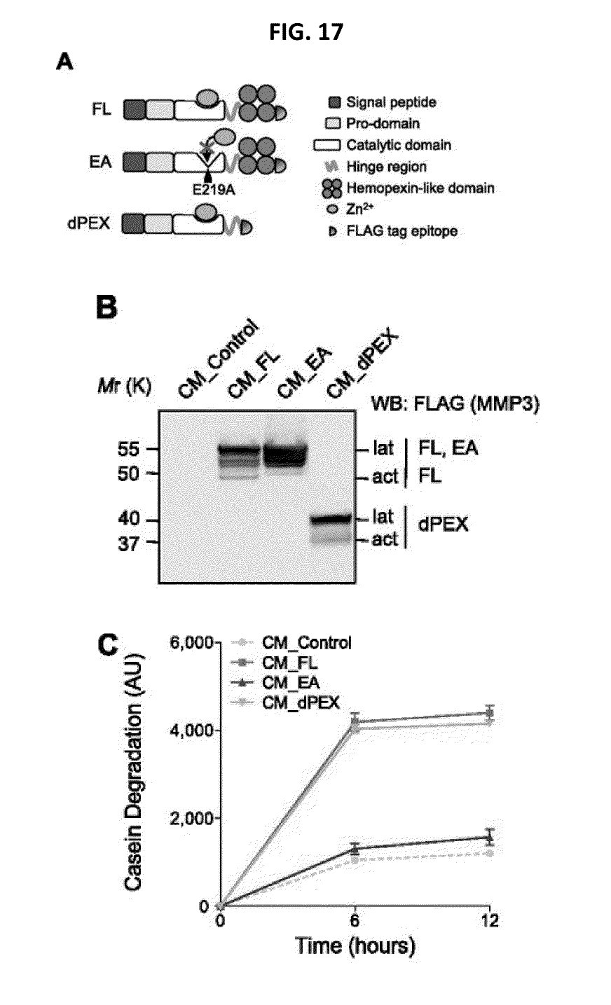

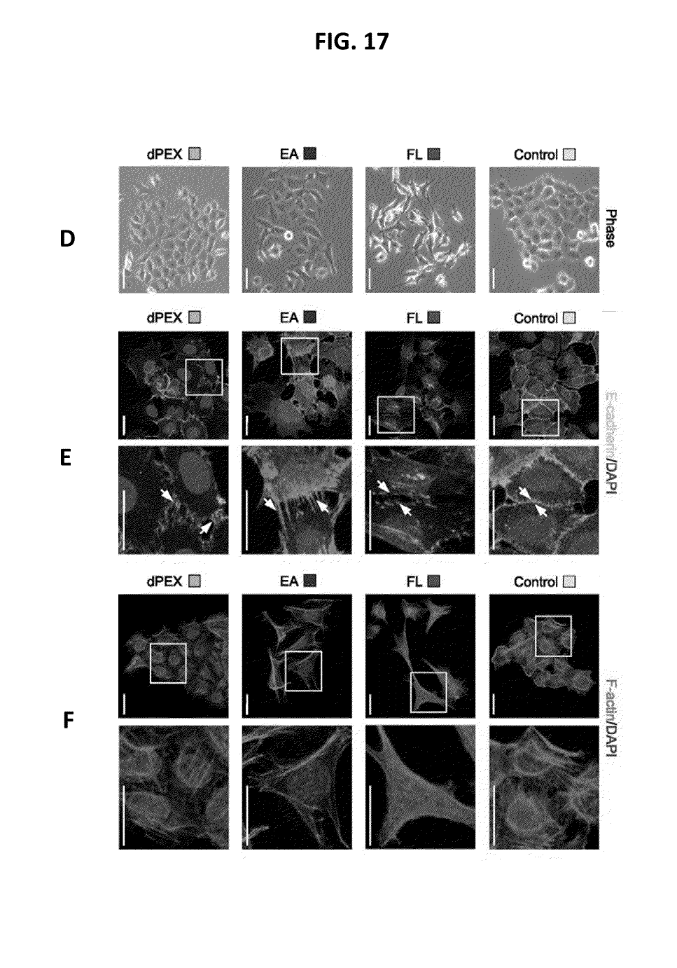

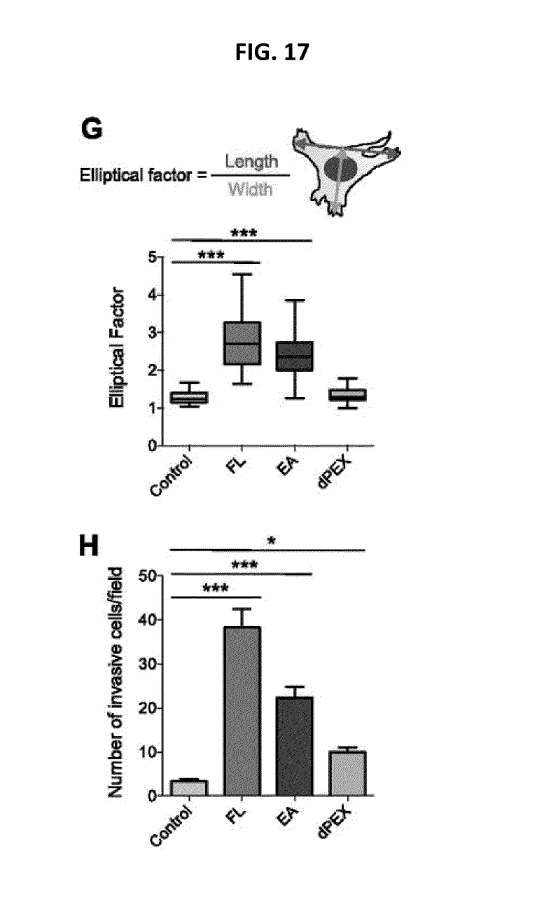

To investigate the function of different domains of MMP3, we engineered three Flag-tagged constructs containing different domains of the MMP3 molecule: the full-length (FL) MMP3, a mutant lacking the hemopexin-like domain (dPEX), and a construct containing a point mutation, E219A (EA), at the catalytic core (FIG. 17A). We overexpressed the distinct MMP3 constructs in SCp2 (FIG. 17B), a mammary cell line shown to undergo EMT upon expression of MMP3 (Lochter et al. 1997a; Radisky et al. 2005). SCp2 cells have a low level of endogenous MMP3 activity that resembles that found in vivo in mammary epithelia; we chose to maintain this activity advisedly to have a positive control for the overexpression of the human homologs in murine cells. This was additionally useful because we observed that the concurrent knockdown of endogenous MMP3 and the introduction of the exogenous levels of the human constructs would lead to aberrant cell behavior. To compare the cultures transduced with different constructs with each other and with the control, we ensured that the endogenous as well as the exogenous levels of MMP3 were comparable in all engineered cell lines (Supplemental FIG. S1). Overexpression of the exogenous constructs in SCp2 showed that the proteolytic activity (measured by casein-quenched degradation) in dPEX was similar to FL and that they both were higher than EA-SCp2 or control cells (FIG. 17C).

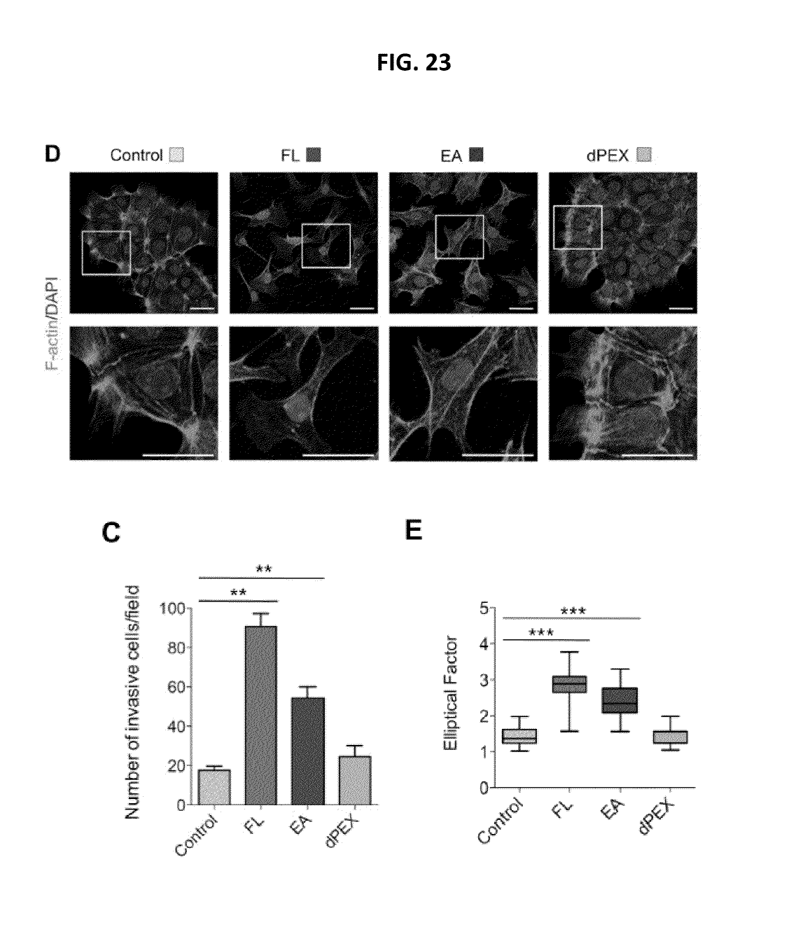

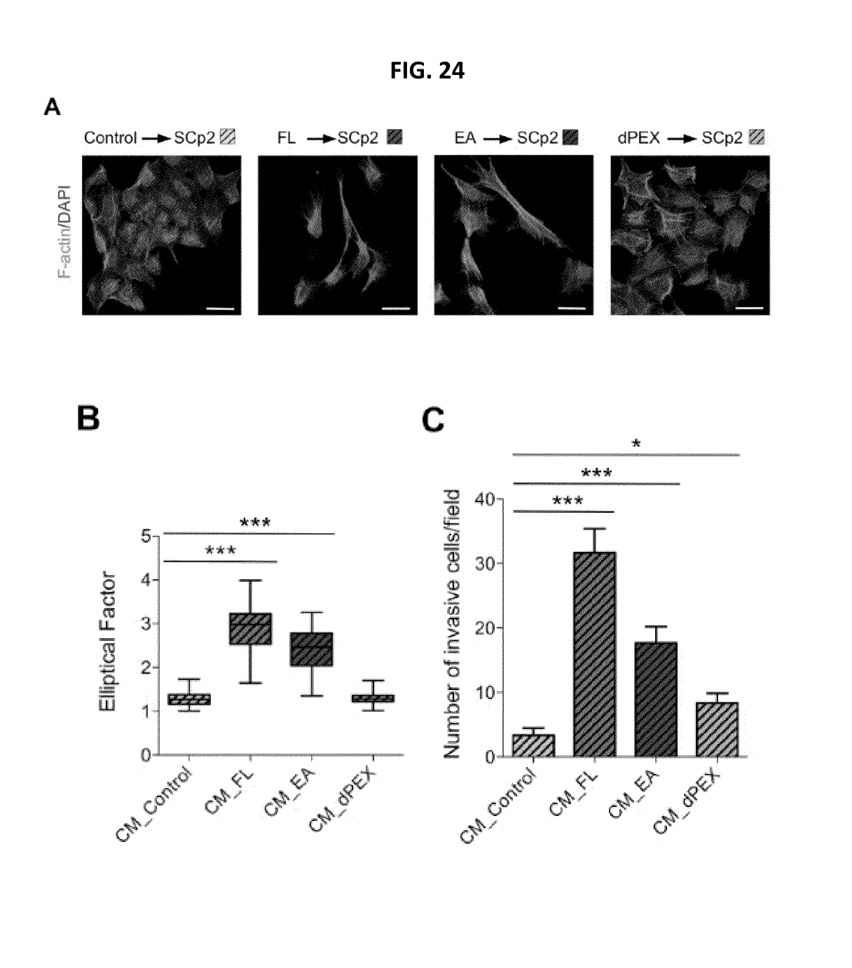

Cell scattering is a functional consequence of EMT (Vincent-Salomon and Thiery 2003); overexpression of FL-MMP3 induced scattering in 2D cultures (FIG. 1D, first and second rows). The EA mutant also stimulated a spindle-shaped morphology and scattered phenotype, albeit to a lower extent (FIG. 17D, third row). In contrast, dPEX-SCp2 did not scatter and resembled the control cultures (FIG. 17D, fourth row). We and others showed that E-cadherin is a substrate for MMP3, and its loss is associated with scattering (Lochter et al. 1997a; Noe et al. 2001). Consistent with these observations, we found that FL and dPEX-MMP3 both reduced the expression of E-cadherin (FIG. 17E, second and fourth rows) by shedding its extracellular domain (Supplemental FIG. 23A). Surprisingly, however, EA-SCp2 cells (which lack the proteolytic activity) still exhibited a stretched phenotype even in the presence of E-cadherin levels similar to control cultures (FIG. 17E, third row), suggesting that the ability of MMP3 to disrupt epithelial morphology was due to activities residing in its other domains.

Using changes in cell morphology and reorganization of filamentous actin (F-actin) as additional endpoints, we observed that in dPEX-SCp2 and control cultures, F-actin was predominantly organized in cortical bundles, and cells had a classical epithelial morphology in 2D (FIG. 17F, first and last rows). In sharp contrast, actin filaments were extended in FL and EA-SCp2 cultures, and cells were elongated (FIG. 17F, second and third rows). We quantified these morphological changes by calculating the ratio of the longest (length) to the shortest (width) axis of the cell, which we refer to as the cellular elliptical factor (FIG. 17G). Whereas FL and EA-SCp2 displayed an elliptical factor >2, cells expressing control vector or dPEX had elliptical factors close to 1. These observations show a critical role for the MMP3 hemopexin domain in altering epithelial cell shape.

Despite the small amount of proteolytic activity of SCp2 cells, these exhibit little invasive behavior (Lochter et al. 1997b); the same is true in SCp2 cells transduced with control vector (FIG. 17H, control). SCp2 transduced with FL-MMP3 had the highest invasive rate, followed by EA and dPEX-SCp2, respectively (FIG. 17H). These data indicate that despite the background proteolytic activity, MMP3 requires the hemopexin domain to induce invasion in SCp2 cells. A similar trend was obtained with EpH4, another mouse mammary epithelial cell line (FIG. 23B-E).

Proteomic Screen Identifies HSP90β as Interacting with the Hemopexin Domain of MMP3

Because MMP3 is a secreted protein, we asked whether the secreted form of this enzyme and its mutants were required to induce the morphological and functional changes observed (FIG. 24). Conditioned medium (CM) from FL-SCp2 was sufficient to induce scattering, elongated shape, and a substantial increase in invasion in parental SCp2 cells. Whereas dPEX-SCp2 CM did not trigger scattering or enhance the elliptical factor, there was a small but significant increase in invasion. However, when the proteolytic activity of the MMP3 construct was ablated (CM from EA-SCp2), there was still a considerable increase in invasion, and cells were elongated. This finding additionally supports the fact that the hemopexin domain is required for invasion in SCp2 cells and raises the question of whether MMP3 functions alone or depends on other factors being present in CM. The hemopexin domain of MMPs is known to interact with other proteins. The MMP14 hemopexin domain was reported to be required for invasion through Col-1 (Tam et al. 2002; Wang et al. 2004) and for binding to the adhesion receptor CD44 and integrin-β1 (Mori et al. 2002, 2013).

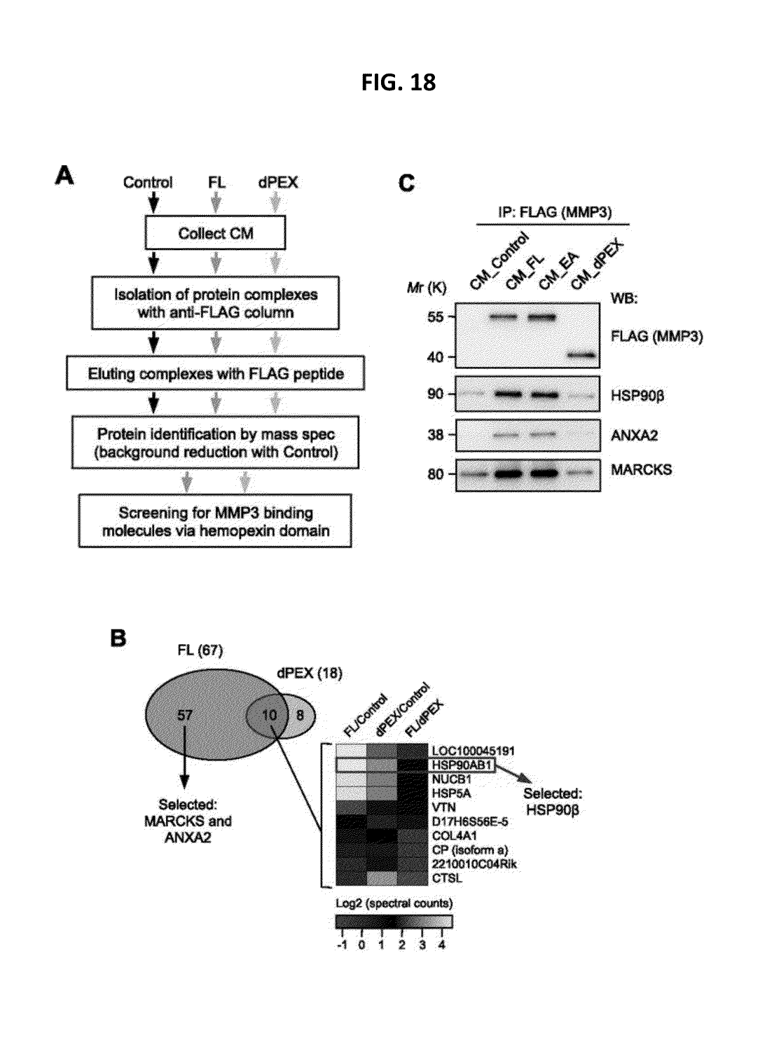

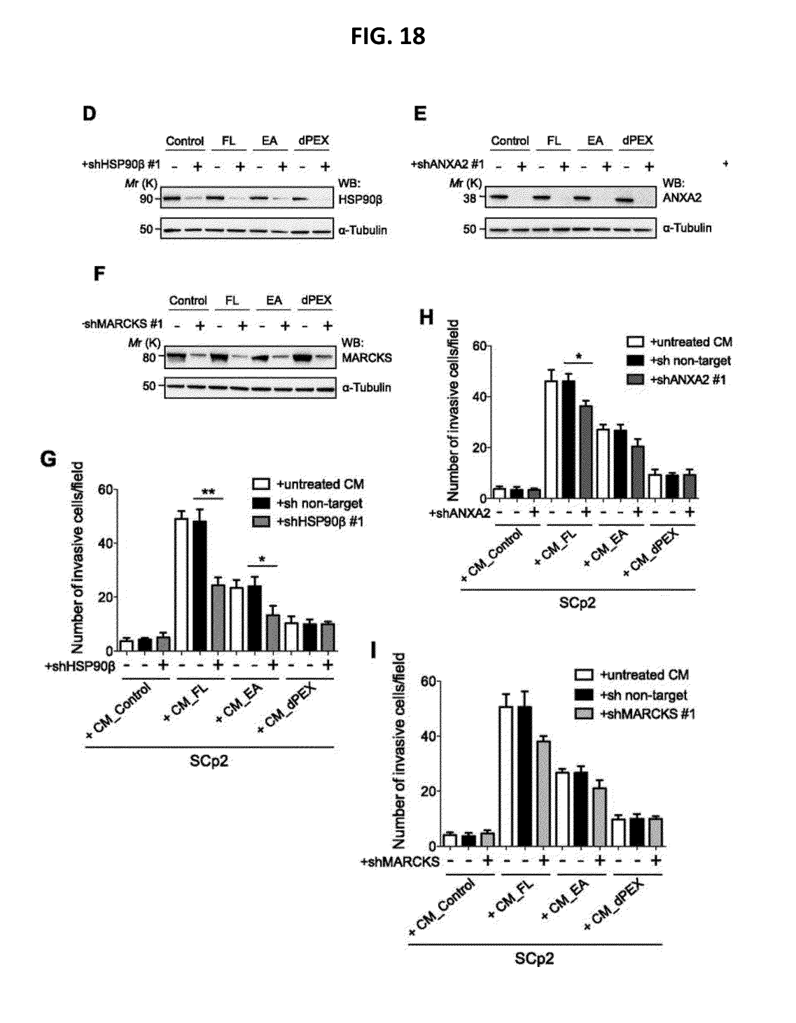

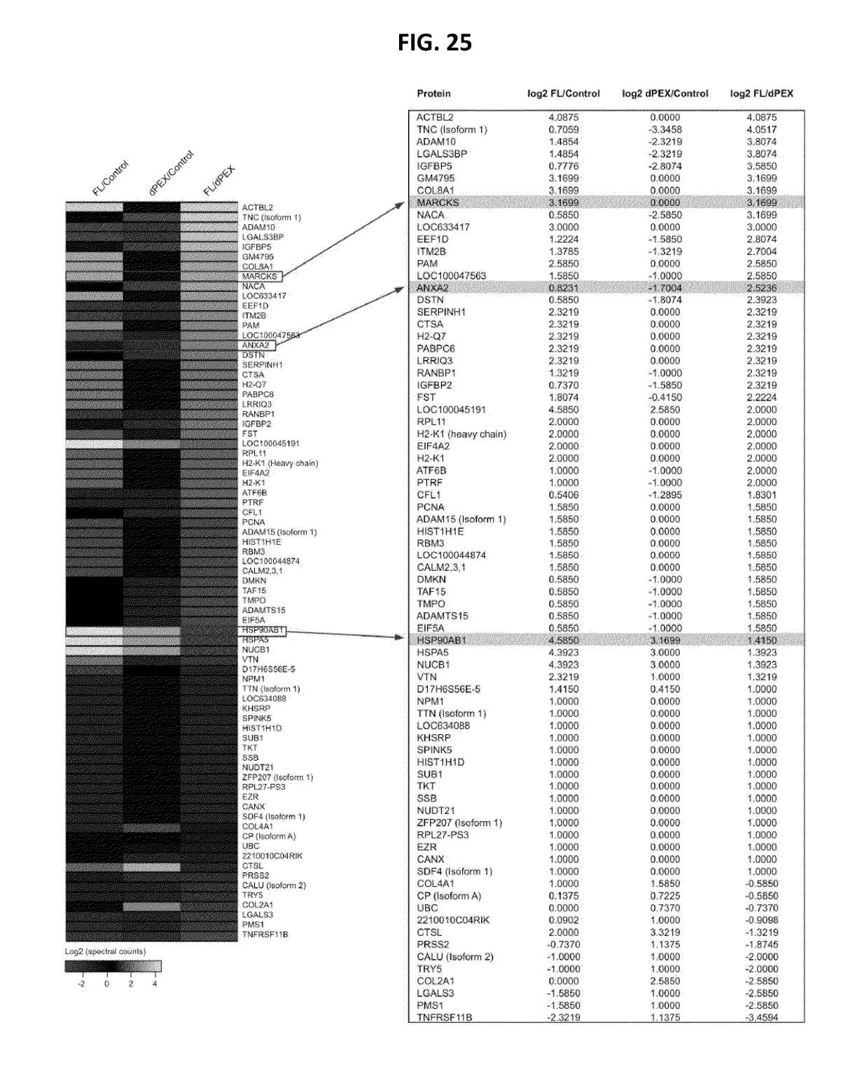

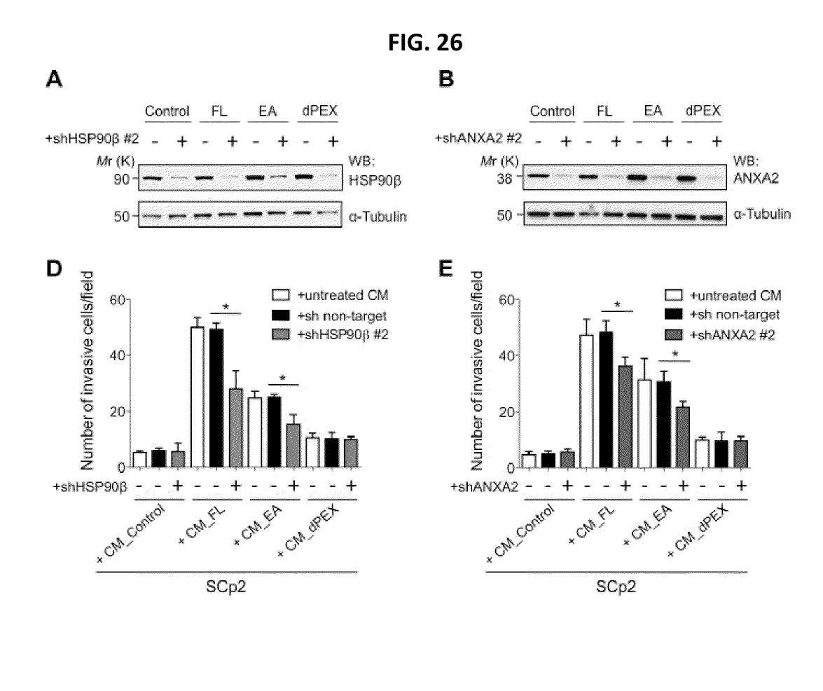

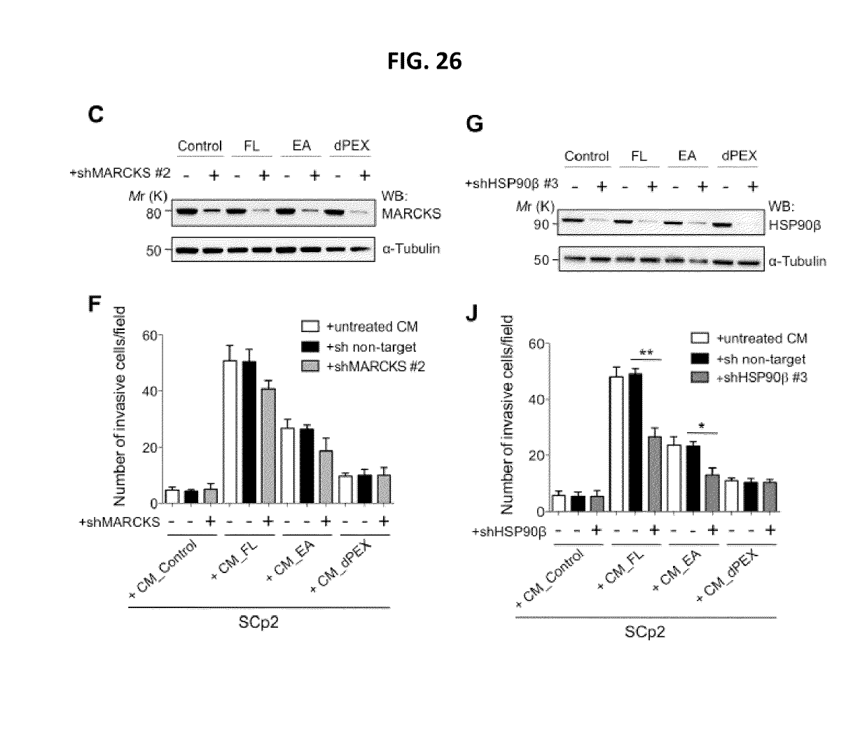

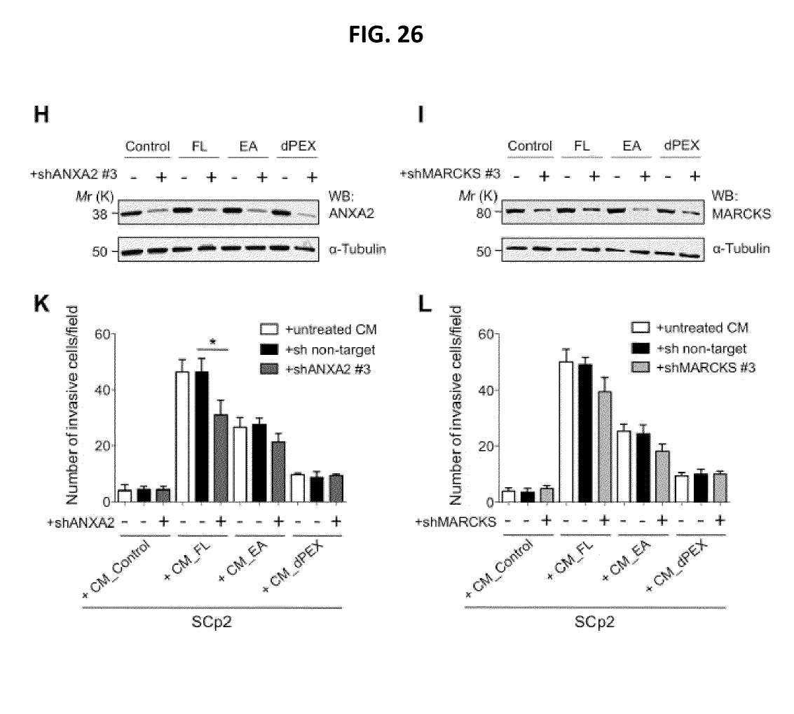

To explore what other factors may be required for the functional activities of MMP3, we isolated Flag-tagged FL or dPEX protein complexes from CM and performed a proteomic analysis to identify proteins that interact with the MMP3 hemopexin domain (FIG. 18A). Based on spectra counts, we selected proteins with abundances >1.5-fold change in FL compared with dPEX (FIG. 18B, left; FIG. 25). Amongst the 75 proteins that passed the selection criteria, we selected myristoylated alanine-rich C-kinase substrate (MARCKS) and annexin A2 (ANXA2), which were previously implicated in regulation of cell shape, motility, and invasion in Xenopus embryos and canine kidney cells (Iioka et al. 2004; de Graauw et al. 2008). Additionally, we selected HSP90β, detected in both FL and dPEX but much higher in FL (FIG. 2B, right). We validated the interaction of the hemopexin domain of MMP3 with these three proteins by coimmunoprecipitation (co-IP) (FIG. 18C).

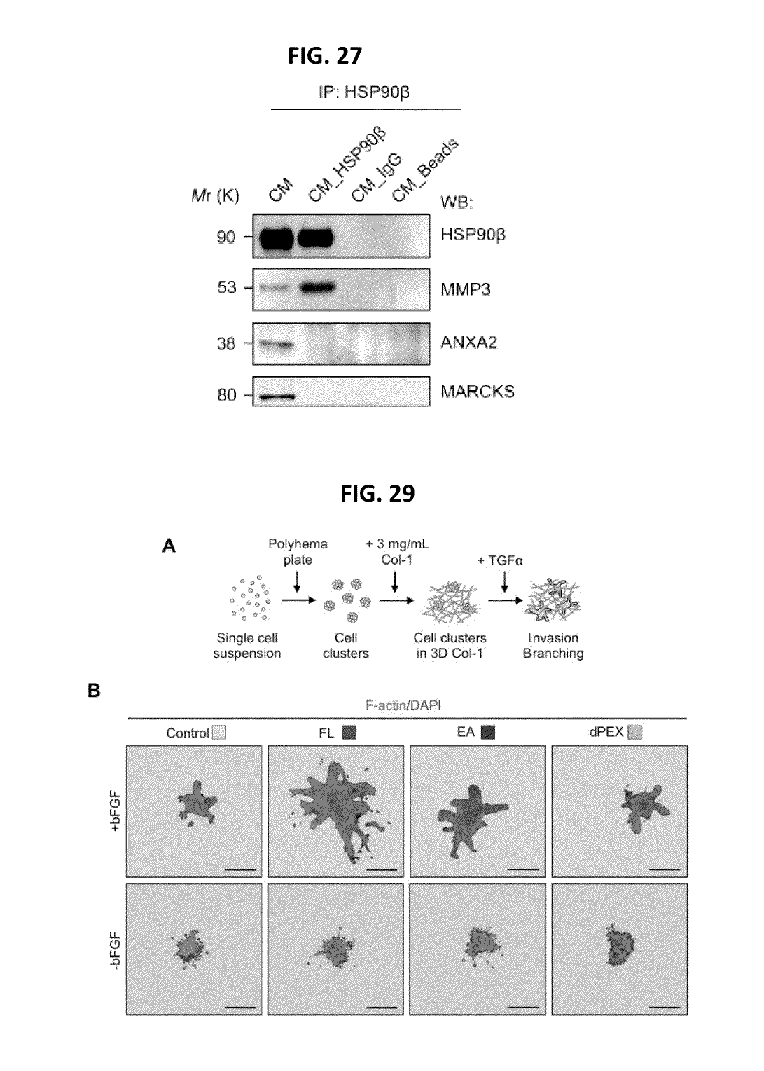

We then asked whether this interaction is functionally significant and required for MMP3-induced invasion. We generated SCp2 cell lines coexpressing each of the MMP3 constructs and either nontargeting shRNA (negative control) or shRNA selectively targeting each of the three proteins (FIG. 18D-F). We treated parental SCp2 with CM from each engineered cell line and screened for invasion using Boyden chambers (FIG. 18G-I). Whereas the knockdown did not affect invasion of cells treated with CM from control or dPEX-SCp2, it significantly reduced the invasiveness of cells treated with FL or EA-SCp2 CM. These results indicate that binding of each one of these three proteins to the hemopexin domain of MMP3 has functional significance, but the inhibition was much more dramatic when HSP90β was inhibited (FIG. 18G). The nature of the complexes containing MMP3 and HSP90β was clarified further by reverse co-IP of HSP90β protein complexes from CM of control SCp2 cells (FIG. 27). Whereas the association of MMP3 and HSP90β was confirmed in reverse, ANXA2 and MARCKS could not be recovered in the immunoprecipitated fraction under these conditions. This suggests that either these proteins do not exist in a single complex at a given time—but may instead represent a network of proteins interacting with one another at different times for different purposes—or the interaction of the other two proteins is weak and thus cannot be detected easily by the reverse co-IP. These data also justify the importance of HSP90β as the major player in regulation of MMP3 function.

### The Levels of Extracellular HSP90β Determine MMP3-Induced Invasion

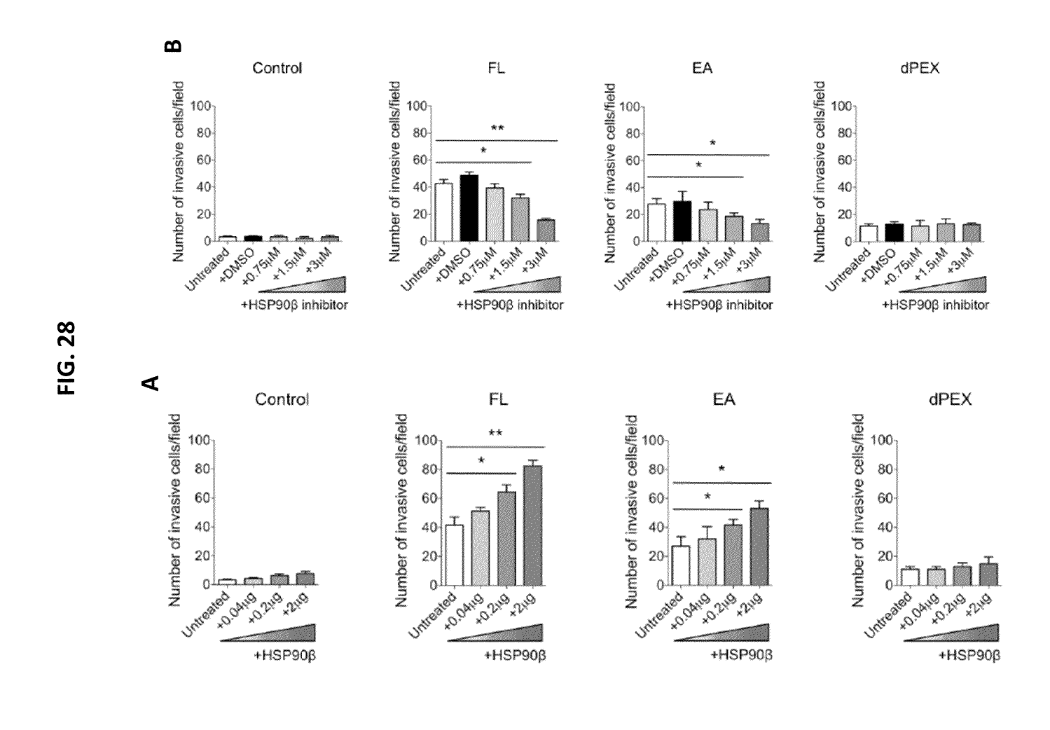

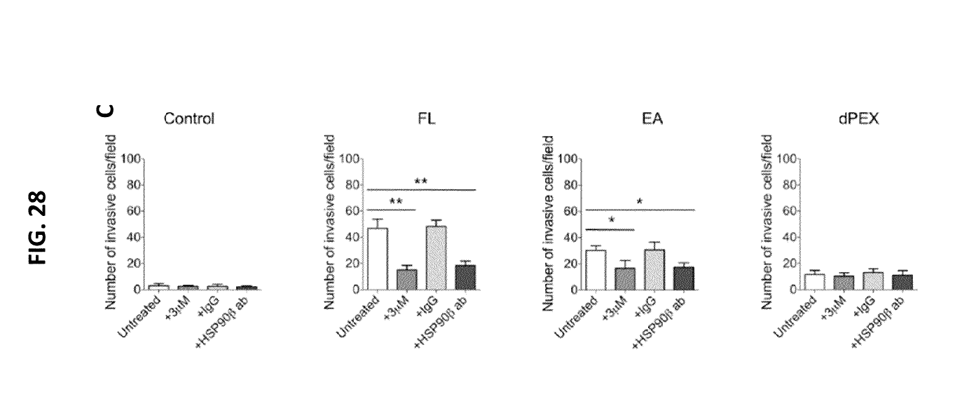

Given the significance of HSP90β in cellular and tissue function, we concentrated on understanding the role of this molecule in regulating MMP3. The levels of HSP90β in each engineered cell line were tuned by adding either a recombinant protein or a specific inhibitor (CCT018159) (Sharp et al. 2007). Increasing HSP90β levels enhanced invasion significantly in FL and EA-SCp2 (FIG. 28A, second and third panels) but did not raise the invasive potential of dPEX-SCp2 or control cells significantly (FIG. 28A, first and last panels). Conversely, inhibition of HSP90β reduced invasion in FL and EA-SCp2 (FIG. 28B, second and third panels) and had no significant effect on dPEX-SCp2 or control cells (FIG. 28B, first and last panels). The above pattern was reproduced when we used a function-blocking antibody against HSP90β and demonstrated that inhibition of extracellular HSP90β was sufficient to reduce invasiveness of FL and EA-SCp2 (FIG. 28C). These data show that MMP3 is unable to perform much of its invasive functions without interacting with HSP90β in the extracellular milieu.

### The Hemopexin Domain is Required for the Invasive Function of MMP3 During Branching Morphogenesis

The finding of the critical role of the hemopexin domain in the invasion function of MMP3 in cell lines needed to be confirmed in a more physiological context. We used two culture models that simulate the normal processes of mammary invasion and branching: primary mammary organoids (FIG. 18A; Simian et al. 2001) and cell clusters of a functionally normal mouse mammary epithelial cell line (EpH4) (FIG. 29A; Hirai et al. 1998; Mori et al. 2013) embedded in Col-1 gels. The physiological relevance of this model is illustrated by the presence of copious amounts of Col-1 in the stroma surrounding epithelial ducts in the murine mammary gland (Williams and Daniel 1983).

There are a number of advantages of using the versatile assay using organoids from the mammary gland. Cell-cell and cell-matrix interactions remain intact, and the architecture of the tissue is not disrupted. Additionally, we can prepare enough mammary organoids from a single mouse (□1200) and infect with the four distinct constructs. Even inbred mice are known to change biochemical and morphological characteristics at different stages of the estrogen cycle as well as in response to handling and context. In this way, we could control for all variations and avoid the excessive use of animals but also achieve statistical significance. Last, we could mark them: The presence of the GFP in the constructs indicated that >80% of the cells were infected. These cultures allow us to not only create a physiological condition where the organoids regenerate an epithelial tree-like structure, but also observe and control extracellular events much more robustly.

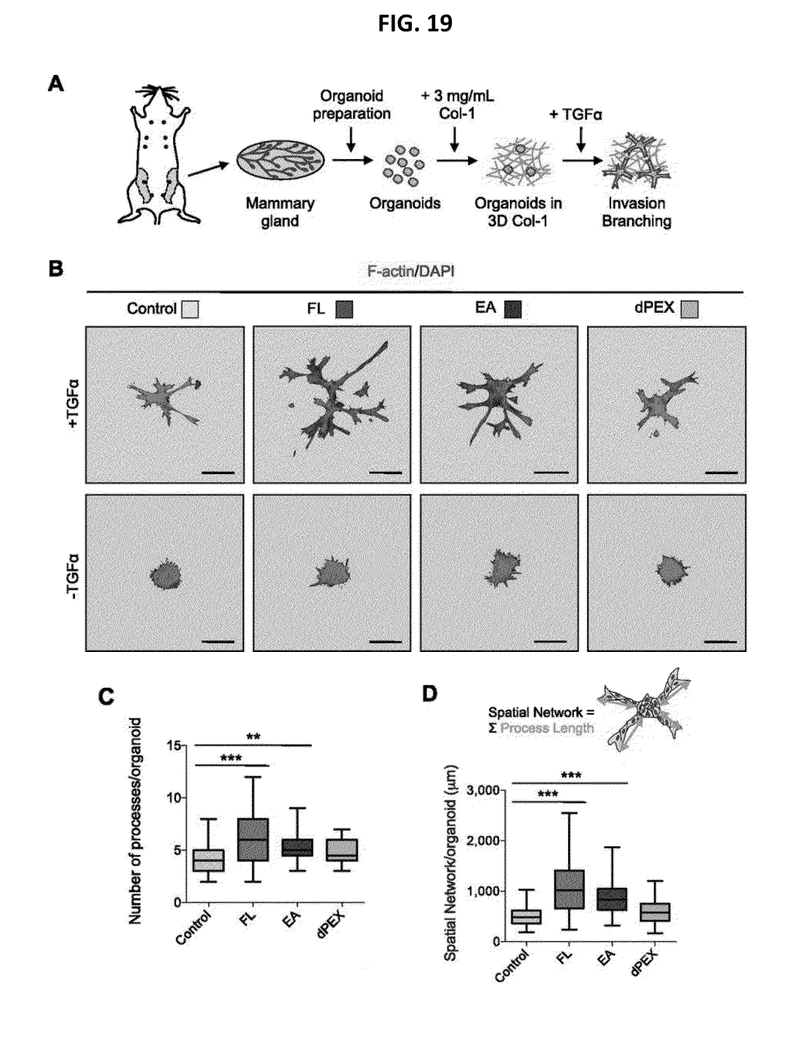

The functional significance of the hemopexin domain was reproduced in our three-dimensional (3D) assays with clustered EpH4 cells (FIG. 29B) and, most importantly, with primary organoids (FIG. 19B). We used two different criteria to quantify invasion and branching of organoids: the number of extended sprouts and processes developed from each structure (FIG. 19C) and the spatial network per organoid (FIG. 19D). As expected, organoids overexpressing FL-MMP3 had the highest number of extending processes and the longest spatial network, indicating that the proteolytic activity would still be necessary if the path is obstructed.

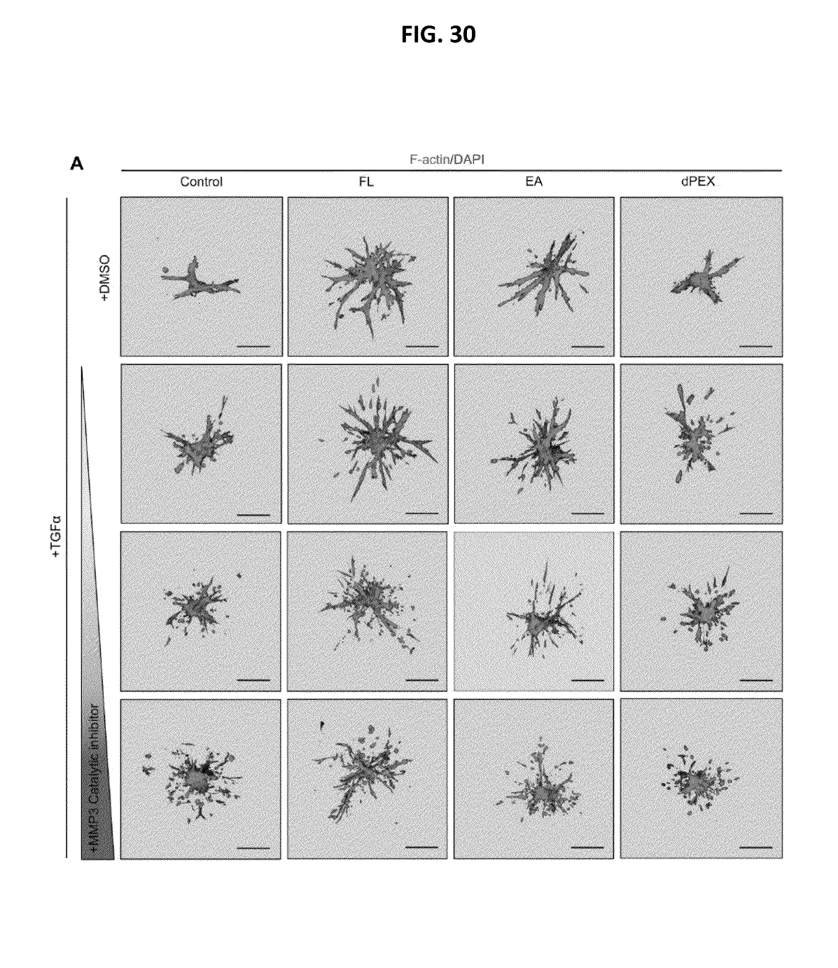

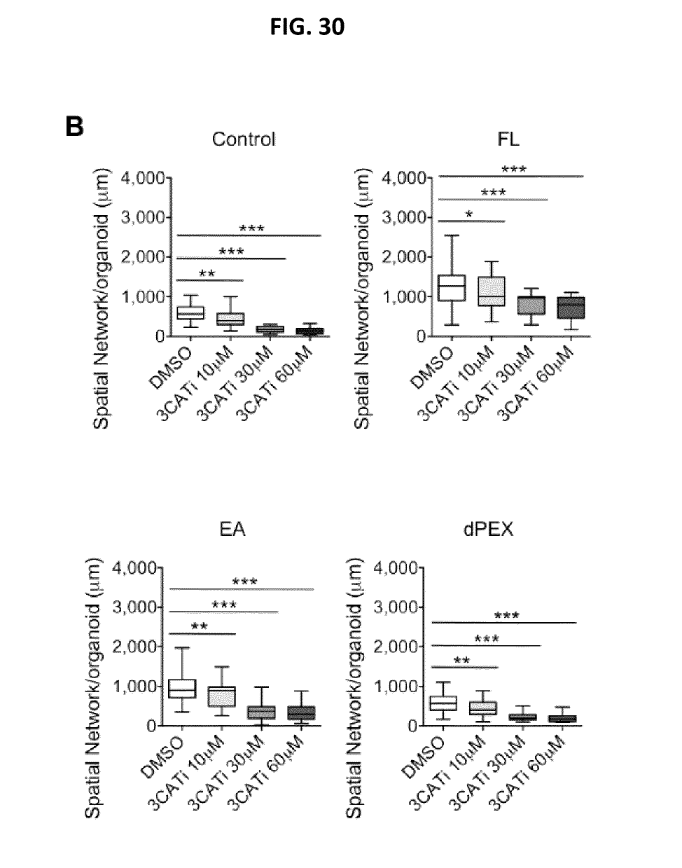

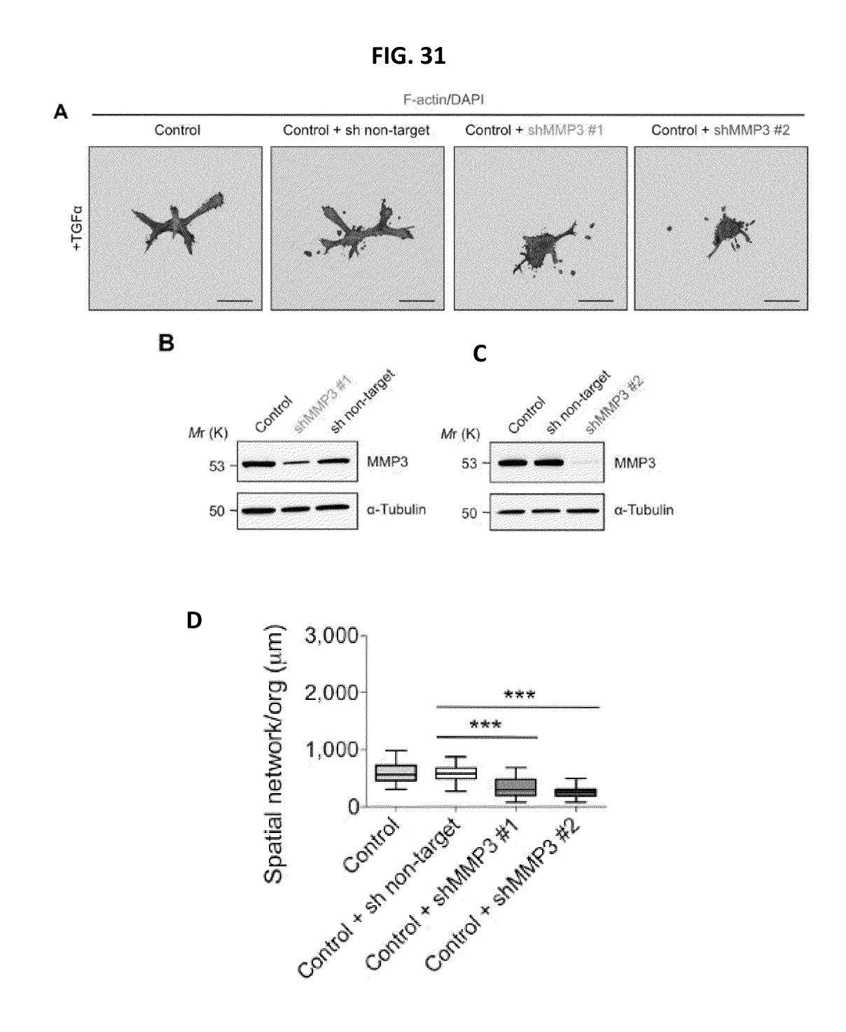

For the purpose of the current experiments, we did not distinguish between branches that were more than one cell layer thick and demonstrated basal and apical polarity and strands that grew as a single file. However, there were very few of the latter in dPEX-overexpressing and control cultures. As mentioned above, we advisedly decided against inhibiting the endogenous MMP3 activity using multiple genetic manipulations because both cells and organoids were sensitive to more than one set of viral infections. We therefore used a peptide that has been shown to inhibit MMP3 proteolytic activity effectively and specifically (Fotouhi et al. 1994; Farina et al. 2002). Inhibition of both endogenous and exogenous MMP3 proteolytic activity decreased branches in a dose-dependent manner in all organoids (Supplemental Fig. S9). Nevertheless, there still was invasion of cells individually or in a single file, with less branching than untreated cultures (FIG. 30A). These data indicate that the hemopexin domain of MMP3 allows epithelial invasion, but in the presence of proteolytic activity, there are more multilayered branches. Additionally, when we knocked down MMP3 in control organoids, there was a significant decrease in invasion and branching (FIG. 30). This reaffirms the requirement for MMP3 for mammary branching morphogenesis and provides an additional reason for our choice of preserving the endogenous MMP3 intact.

The Interaction of HSP90β with MMP3 is Required for Invasion

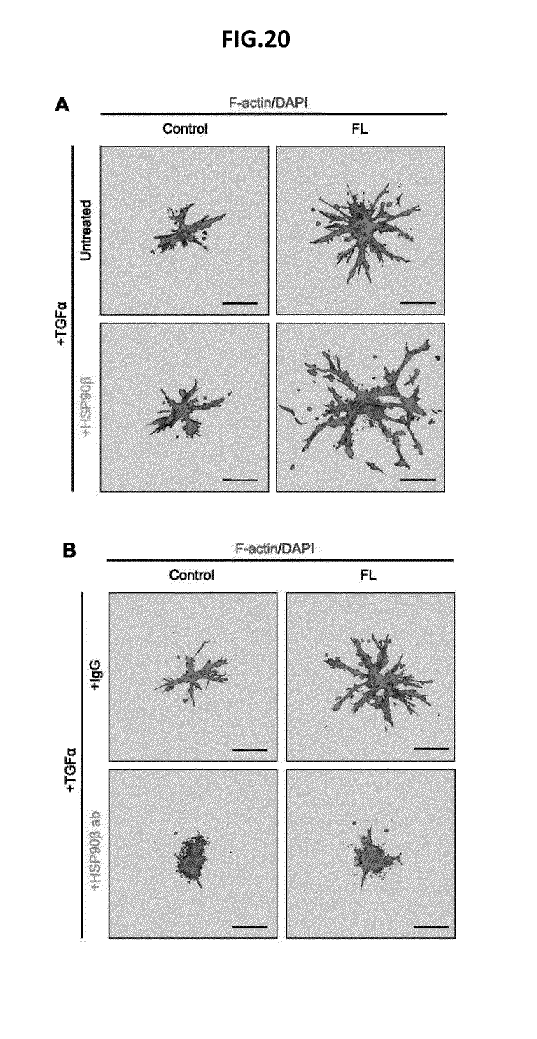

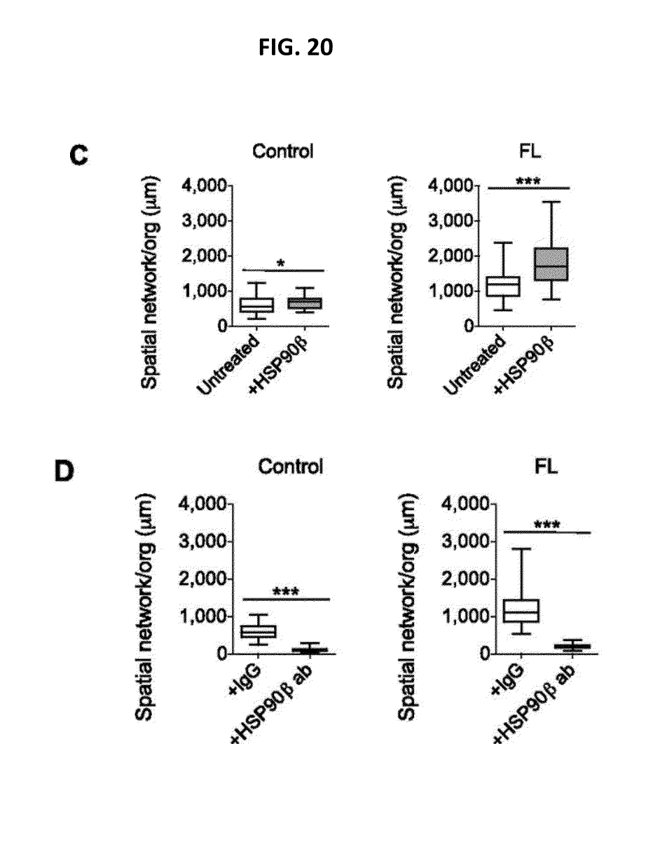

Having shown the relevance of the distinct domains for invasion and branching also in organoids, we examined the requirement of HSP90β in organoids transduced with different constructs receiving either recombinant protein (FIG. 20A) or a function-blocking antibody against HSP90β (FIG. 20B). The recombinant HSP90β added extracellularly enabled the secreted MMP3 to induce the most exuberant branched structures (FIG. 20A, bottom right) and the longest spatial network observed so far (FIG. 20C, right). Importantly, blocking the extracellular HSP90β with inhibitory antibodies added to the medium abolished branching ability in all organoids, including controls (FIG. 20B [bottom], D). Organoids receiving the construct with a deleted hemopexin domain were essentially identical to the controls. Additionally, there was very little co-IP of MMP3 with HSP90β in the absence of exogenous HSP90β (data not shown). These findings identify the crucial role of extracellular HSP90β in mammary epithelial invasion and branching, with binding occurring in the presence of the hemopexin domain of MMP3.

### Discussion

The importance of MMPs for sculpting the architecture of branched organs is well accepted. This statement is demonstrated in particular in the mammary gland. We and others showed that overexpression of MMP3 in mammary epithelia enhanced lateral branching and precocious alveolar development in virgin mice (Sympson et al. 1994; Witty et al. 1995). These mice eventually developed tumors that exhibited chromosomal aberrations (Sternlicht et al. 1999) through a mechanism dependent on ROS and RAC1B, a spliced variant of RAC1 (Radisky et al. 2005). Conversely, we showed that MMP3 controls lateral branching in vivo (Wiseman et al. 2003) and in Col-1 gels (Simian et al. 2001).

In many of these experiments, we and others had assumed that the catalytic domain of MMP3 was responsible for these functions. More recently, there has been some biochemical evidence that the hemopexin domain of some MMPs has a role in the nonproteolytic function. Mori et al. (2002) and Dufour et al. (2008) examined the role of the hemopexin domain of MMP14 and MMP9 in cancer cells and fibroblasts, respectively, and showed that it is necessary for cell migration. Likewise, the hemopexin domain, but not the catalytic activity, of MMP12 was shown to be required for the antimicrobial function of this enzyme (Houghton et al. 2009). The only clear evidence for the physiological relevance of the hemopexin domain in vivo came from a report by Glasheen et al. (2009) in Drosophila; these investigations showed that whereas the catalytic domain was still required for all MMP functions, the hemopexin domain was specifically implicated in invasion during metamorphosis.

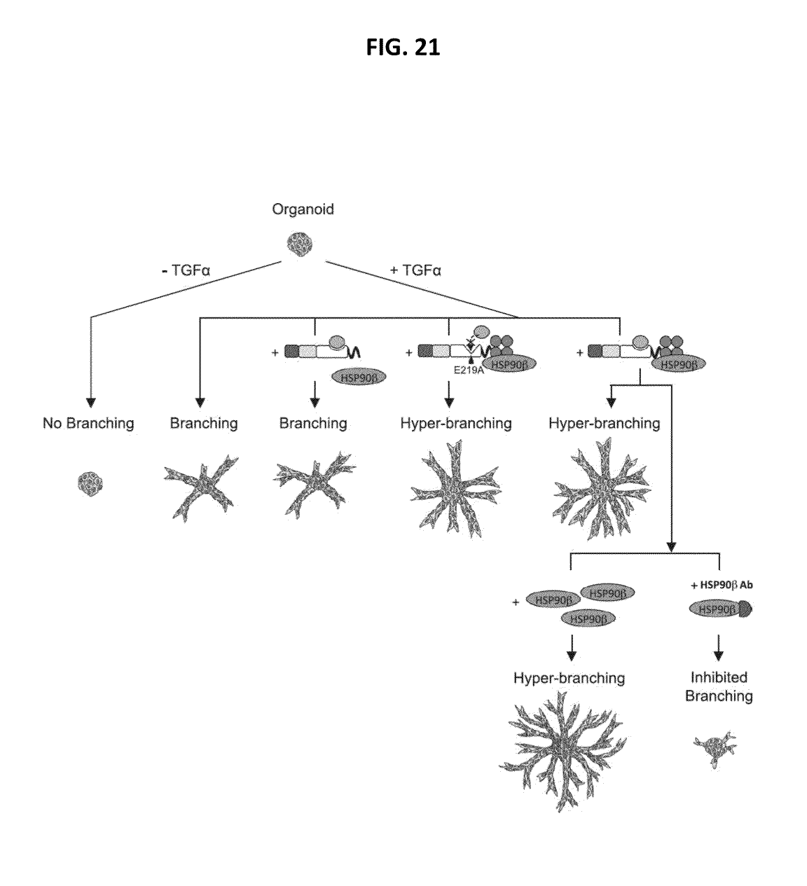

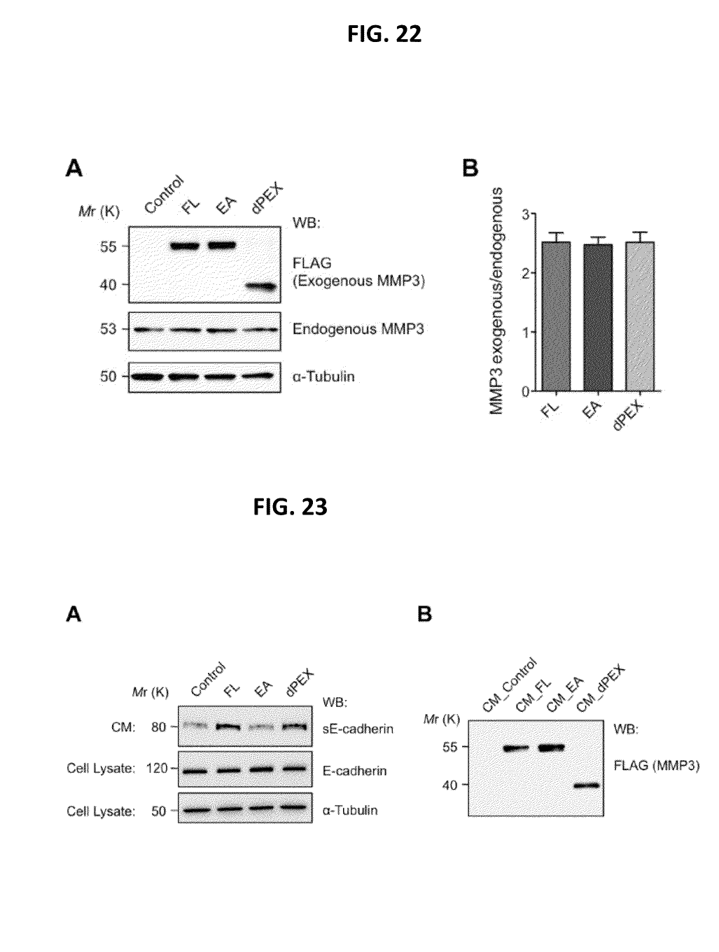

Neither the requirement for the hemopexin domain of MMP3 nor the surprising interaction with extracellular HSP90β were known or reported previously. Here we show that cells and tissues that overexpress MMP3 but lack catalytic activity can invade and branch easily in 3D Col-1 gels. Additionally and importantly, we show that the functional activity of the hemopexin domain of MMP3 requires extracellular interaction with HSP90β (FIG. 21).

The previous literature on functions of HSP90 place its activity essentially within the cell, where it works as a “hub of protein homeostasis” by facilitating the maturation of a wide range of proteins (Taipale et al. 2010). It is only with regard to HSP90α that the extracellular function has been mentioned. A number of investigators have shown that the a isoform of HSP90 is present in CM of either cancer cells or “wounded cultures” (Eustace et al. 2004; Li et al. 2007; Cheng et al. 2008). Our discovery that the extracellular HSP90β is essential for MMP3-driven invasion and branching adds a new dimension to this chaperone's functions. Despite the fact that HSP90α and HSP70, which was shown previously to increase the association between MMP2 and HSP90α in vitro (Sims et al. 2011), are present intracellularly in our model, they are not found in the extracellular milieu (data not shown). That HSP90β has a crucial extracellular function was shown by addition of specific inhibitory antibodies to the medium, resulting in complete inhibition of branching (FIG. 20B,D). These data indicate that the presence of HSP90β in the medium is a selective process and is not due to cell lysis or apoptosis.

Mice deficient for HSP90β fail to develop a placental labyrinth and die around mid-gestation (Voss et al. 2000). This fact prevented us from characterizing their mammary gland development in vivo. Additionally, despite the fact that Mmp3-null mice are viable and fertile, they compensate the reduced secondary and tertiary branching phenotype by day 70 (Wiseman et al. 2003). The use of ECM gels, however, has allowed us to elucidate the role of different domains of MMP3 as well as prove that extracellular HSP90β regulates MMP3 function in invasion and branching through interaction with the hemopexin domain. The primary organoids develop into hundreds of minimammary epithelial trees, thus offering a model of mammary epithelial development in a robust and manipulable format.

The signaling pathways and regulatory mechanisms that drive branching in mammalian organs have been described by a number of laboratories, including ours, and involve multiple members of the receptor tyrosine kinase (RTK) family (for review, see Lu and Werb 2008). Sustained activation of MAPK^{ERK-1,2} in response to hepatocyte growth factor was shown to be required for kidney epithelial morphogenesis in Col-1 gels (Maroun et al. 2000). We showed that the MAPK^{ERK-1,2} pathway also integrates distinct and antagonistic signals from TGFα and FGF7 to determine the final morphogenetic response of mammary organoids cultured in 1rECM; sustained MAPK activation downstream from TGFα and EGFR induces branching, whereas its transient activation downstream from FGF7 and FGFR2 stimulates proliferation but not branching (Fata et al. 2007). FGF7 acts in part by suppressing the expression of MMP3, and inhibition of the latter reduces branching significantly both in culture and in vivo (Simian et al. 2001; Wiseman et al. 2003). Our discovery that extracellular HSP90β is critical for MMP3 function in invasion and branching places HSP90β as an important player in the signaling pathways that determine the final mammary morphogenetic fate.

The presence of HSP90 in murine mammary glands was reported in 1989 (Catelli et al. 1989); therefore, it is surprising that its role in functional and morphogenetic aspects of the mammary gland is still poorly understood. The fact that HSPs have been postulated as molecular chaperones that mitigate the life-threatening effects of heat and other stresses on the proteome (Taipale et al. 2010) poses the question of whether HSP90β may also play a role in stabilization and maturation of MMP3. We are now beginning to understand that HSP90's functions extend well beyond stress tolerance, and associated changes in its clients can then exert marked effects on the relationship between genotype and phenotype, influencing human health, disease, and evolutionary processes (Rutherford and Lindquist 1998; Queitsch et al. 2002; Cowen and Lindquist 2005). The presence of HSP90β in the medium and the functional significance of its interaction with MMP3 are further proof that HSP90-mediated events are above and beyond the heat-shock response. Our preliminary data indicate that the extracellular source of HSP90β for luminal epithelial branching most probably is the myoepithelial cells in vivo (data not shown). These data, combined with some evidence that MMP3 is mainly produced by stromal fibroblasts (Witty et al. 1995; Kouros-Mehr and Werb 2006), raise the exciting possibility that extracellular interaction of HSP90β with MMP3 may be a way for different cell types to communicate in the coordination of the normal processes of invasion and branching.

In the initial mass spectrometry data, we found many additional molecules that appear to be interacting with MMP3. In particular, we show that ANXA2 and MARCKS were coimmunoprecipitated with MMP3, with binding occurring in the presence of the hemopexin domain. Our preliminary data also showed that depletion of each of these proteins reduced invasiveness in SCp2 cells. Unlike the interaction between HSP90β and MMP3 that happened in both directions, the reverse co-IP of ANXA2 and MARCKS with HSP90β could not be confirmed under these conditions. In addition, our proteomic screen identified other proteases such as ADAM10, ADAMTS15, and Cathepsins A and L as possible proteins that may interact extracellularly with MMP3 (FIG. 25). The functional significance of these latter proteins remains to be determined. Our data from the mass spectrometry, however, tentatively suggest that a cascade of proteases might function collectively to orchestrate epithelial invasion.

Finally, we showed most recently that the signaling module for MMP14, a membrane-bound MMP, in branching of the end bud of the mammary gland of virgin mice is its transmembrane/cytoplasmic domain in conjunction with integrin-β1 (Mori et al. 2013). Thus, the findings presented here, along with the above work, may provide a compelling explanation for why inhibitors of MMPs failed so dramatically in the clinic (Overall and Kleifeld 2006). Targeting noncatalytic sites of MMPs as well as the interacting partners with agents such as small inhibitors or antibodies for the binding sites of integrin-β1 and HSP90β may yield more effective and tissue-specific inhibitors.

### Materials and Methods

### Restriction Enzymes, Antibodies, Proteins, and Chemical Reagents

All restriction enzymes were acquired from New England Biolabs. Bovine dermis acid-solubilized Col-1 solution (IAC-50) was purchased from Koken. Antibodies against the following proteins were obtained as indicated: Flag (F1804, M2, Sigma; 1:500 for Western blotting), E-cadherin (13-1900, clone ECCD-2, Invitrogen; 1:1000 for Western blotting; 1:200 for immunofluorescence), HSP90β (5087, Cell Signaling; 1:1000 for Western blotting), HSP90β (NBP1-61773, Novus Biologicals; 40 μg/mL for function-blocking experiments; 10 μg for co-IP experiments), MARCKS (P0370, Sigma; 1:1000 for Western blotting), ANXA2 (AF3928, R&D Systems; 1:1000 for Western blotting), MMP3 (ab18898, Abcam; 1:1000 for Western blotting), α-tubulin (T6074, clone B-5-1-2, Sigma; 1:5000 for Western blotting), and rabbit IgG (2729, Cell Signaling; 40 μg/mL for function-blocking experiments). Alexa Fluor 594 Phalloidin (A12381, Molecular Probes; 1:400) was used to stain F-actin. DAPI (Sigma) was used to stain nuclei. HSP90β inhibitor CCT018159 (385920), MMP3-specific peptide-based inhibitor (444218), and recombinant HSP90β (385903) were purchased from Calbiochem/EMD Millipore.

### Construction of Expression Plasmids

All MMP3 mutants were constructed using a PCR-based method. The cDNA sequence used as a template was cloned from a human breast cell line and sequence-confirmed by comparison with gene accession number NM_{—}002422.3. FL contains the full-length MMP3 cDNA. EA is a catalytically inactive mutant, holding a point mutation E219A at the catalytic core. dPEX is a hemopexin-like domain-deleted mutant (ΔN289-C477). A mammalian expression vector, pCDH-EF1-MCS-T2A-copGFP (System Biosciences), was used to express the gene products. To detect MMP3 protein, the Flag epitope (Asp-Tyr-Lys-Asp-Asp-Asp-Asp-Lys (SEQ ID NO:18)) was inserted at the C terminus of every construct generated. All cDNA constructs were confirmed by DNA sequencing.

To generate FL and dPEX constructs, PCR fragments flanked by EcoRI/BamHI restriction enzyme digest sites at the 5′ and 3′ ends, respectively, were obtained using the same sense primer (5′-CGTTACGAATTCATGAAGAGTCTTCCAATCCTACTG-3′) (SEQ ID NO:19) and different antisense primers: for FL, 5′-CGAATCGGATCCCTT GTCATCGTCGTCCTTGTAGTCACAATTAAGCCAG-3′ (SEQ ID NO:20); and for dPEX, 5′-CCTGCAGGATCCCTTGTCATCGTCGTCCTTGTAGTCGTTGGCTGGCGTC-3′ (SEQ ID NO:21). To create the EA construct, two PCR-fragments were first generated using two different primer sets: PCR1, sense primer, 5′-CGTTACGAATTCATGAA GAGTCTTCCAATCCTACTG-3′ (SEQ ID NO:22); PCR1, antisense primer, 5′-AGCAACAAGAAATAAATTGGTCCCTGTTG-3′ (SEQ ID NO:23); PCR2, sense primer, 5′-GTTGCTGCTCATGCCATTGGCCACTCCCTG-3′ (SEQ ID NO:24); and PCR2, antisense primer, 5′-CGAATCGGATCCCTTGTCATCGTCGTCCTTGTAGTCACAATTAAGCCAG-3′ (SEQ ID NO:25). These fragments were then linked together (using 5′-CGTTACGAATTCATGAAGAGTCTTCCAATCCTACTG-3′ (SEQ ID NO:26) and 5′-CGAATCGGATCCCTTGTCATCGTCGTCCTTGTAGTCACAATTAAGCCAG-3′ (SEQ ID NO:27) as sense and antisense primers, respectively), generating the final PCR-fragment encompassing the point mutation E219A. After EcoRI/BamHI digestion, FL, dPEX and EA products were ligated into EcoRI/BamHI digested pCDH-EF1-MCS-T2A-copGFP.

### Lentiviral Production and Concentration.

293FT packaging cells (Invitrogen) were transfected with plasmids carrying FL, dPEX, EA, control vector or shRNA constructs using FuGENE6 (Roche), according to the manufacturer's instructions. Transfected cells were cultured in DMEM high glucose containing 10% FBS, 100 U/ml penicillin, 100 □g/mL streptomycin, 0.1 mM MEM Non-Essential Amino Acids, 6 mM L-glutamine and 1 mM MEM Sodium for 24 h, after which the medium was replaced with fresh one. Viral supernatant was collected 48 h later, filtered with 0.45 □m filters, concentrated using Lenti-X Concentrator (Clontech), aliquoted and stored at −80 □C until use

shRNA-Mediated Knockdowns

shRNA constructs selectively targeting HSP90β, ANXA2, MARCKS, or MMP3 were purchased from MISSION shRNA library (Sigma) (sequences are detailed in Table 1 below. Control cells were infected with nontargeting shRNA (SHC002, Sigma). Knockdown efficiency was verified by Western blotting with the appropriate antibodies.

TABLE 1
Detailed sequences of distinct shRNAs used in
the experiments.

| | SEQ | |
|---|---|---|
| shRNA | ID NO: | Sequence (5′-3′) |
| HSP90β #1 | 40 | CCGGCAGGAGGAGTATGGCGAATTCTCGA |
| | | GAATTCGCCATACTCCTCCTGCTTTTTG |
| HSP90β #2 | 41 | CCGGCATGGAAGAGGTGGATTAAAGCTCG |
| | | AGCTTTAATCCACCTCTTCCATGTTTTTG |
| HSP90β #3 | 42 | CCGGGCTGAACAAGACAAAGCCTATCTCG |
| | | AGATAGGCTTTGTCTTGTTCAGCTTTTT |
| ANXA2 #1 | 43 | CCGGGTATGATGCTTCGGAACTAAACTCG |
| | | AGTTTAGTTCCGAAGCATCA |
| | | TACTTTTTG |
| ANXA2 #2 | 44 | CCGGGAGCATCAAGAAAGAGGTCAACTCG |
| | | AGTTGACCTCTTTCTTGATG |
| | | CTCTTTTTG |
| ANXA2 #3 | 45 | CCGGCGAGACAAGGTCCTGATTAGACTCG |
| | | AGTCTAATCAGGACCTTGTC |
| | | TCGTTTTTG |
| MARCKS #1 | 46 | CCGGCTTCTCCTTCAAGAAGAGCAACTCG |
| | | AGTTGCTCTTCTTGAAGGAG |
| | | AAGTTTTTG |
| MARCKS #2 | 47 | CCGGGCCAAGATAATATGCCACTAACTCG |
| | | AGTTAGTGGCATATTATCTT |
| | | GGCTTTTTG |
| MARCKS #3 | 48 | CCGGCTCCTCCACGTCGTCGCCCAACTCG |
| | | AGTTGGGCGACGACGTGGAG |
| | | GAGTTTTTG |
| MMP3 #1 | 49 | CCGGCAAGATGATGTAGATGGTATTCTCG |
| | | AGAATACCATCTACATCATCTTGTTTTTG |
| MMP3 #2 | 50 | CCGGCCCACATATTGAAGAGCAATACTCG |
| | | AGTATTGCTCTTCAATATGTGGGTTTTTG |
| Non- | 51 | CCGGCAACAAGATGAAGAGCACCAACTCG |
| targeting | | AGTTGGTGCTCTTCATCTTGTTGTTTTT |

### Cell Culture and Transduction

SCp2 cells were cultured in Dulbecco's modified Eagle's medium/Ham's F-12 nutrient mixture (DMEM/F-12) supplemented with 5% fetal bovine serum (FBS), 5 μg/mL insulin, and 50 μg/mL gentamicin and maintained as previously described (Desprez et al. 1993). EpH4 cells were cultured in DMEM/F-12 medium supplemented with 2% FBS, 5 μg/mL insulin, and 50 μg/mL gentamicin and maintained as previously described (Reichmann et al. 1989). For transduction, cells were seeded in 24-well plates (1×10⁵ cells per well) and infected with lentiviral particles carrying different expression plasmids using MISSION ExpressMag Beads (Sigma) according to the manufacturer's instructions. Cells transduced with lentivirus carrying shRNA constructs were additionally selected with 2 μg/mL puromycin.

### Preparation of Primary Mammary Organoids and Transduction

Primary epithelial organoids were isolated from 8-wk-old virgin FVB mice as previously described (Fata et al. 2007). Briefly, inguinal glands were removed, minced with two parallel razor blades, and gently shaken for 30 min at 37° C. in a 50-mL collagenase/trypsin mixture (0.2% trypsin, 0.2% type-IV collagenase, 5% FBS, 5 μg/mL insulin in DMEM/F-12). After centrifugation at 80 g for 10 min, supernatant was discarded, and the cell pellet was resuspended in DMEM/F-12. The suspension was pelleted again, resuspended in 4 mL of DMEM/F-12 containing 80 U of DNase I (Sigma), and incubated for 5 min at room temperature with occasional shaking. After the suspension was spun at 80 g for 10 min, a series of differential centrifugations in DMEM/F-12 was implemented to separate the epithelial organoids from single cells, fibroblasts, and fibrillar extracellular matrices. The final pellet was resuspended in the desired amount of medium. For transduction, organoids were seeded in 24-well polyhema-coated plates (1000 organoids per well) and infected with lentivirus in the presence of 8 μg/mL polybrene for 24 h.

### Preparation of Cell Clusters and Transduction

EpH4 cells suspended in growth medium were plated in six-well polyhema-coated plates (1×10⁵ cells per well) and incubated overnight at 37° C., yielding rounded clusters. Single cells were removed by differential centrifugation, and the final pellet was resuspended in the desired amount of medium.

### Branching Morphogenesis Assay

Primary organoids or clustered EpH4 cells were embedded in 3D Col-1 gels as previously published (Simian et al. 2001; Mori et al. 2013). In brief, acid-solubilized Col-1 solution was mixed gently on ice with 1 vol of 10×DMEM/F-12 (pH adjusted to 7.4 with 0.1 M NaOH), and the concentration was adjusted to 3 mg/mL with DMEM/F-12. A basal layer of 80 μL of Col-1 was poured into each well of an eight-well chambered coverglass (155409, Thermo Scientific) and allowed to gel for 5 min at 37° C. A second layer of 200 μL of Col-1 containing 150 organoids or EpH4 clusters was added to each well and placed immediately at 37° C. After gelation, 400 μL of chemically defined medium (DMEM/F-12 containing 1% insulin/transferrin/selenium, 1% penicilin/streptomycin) with 9 nM TGFα (Sigma) or 9 nM bFGF (Sigma) was added to each well (unless stated otherwise) and replaced every other day.

After 3 d of culture, gels were fixed with 4% formalin for 30 min and stained with phalloidin and DAPI for 1 h. Structures were imaged with an upright Zeiss LSM710 using a 0.8 NA 20× air objective. An organoid or cell cluster was defined as invading and branching when it had at least three independent extending processes that were at least half the diameter of the center of the organoid or cell cluster. The number of extending processes and their average length were determined using the Imaris program (Bitplane). We defined a new metric of invasion and branching, which we refer to as the spatial network per organoid. This is defined as the sum of the length of all of the extending processes developed from each organoid. Fifty structures were counted per condition, and the experiments were executed at least three times.

### Caseinase Activity Assay

CM was incubated with a casein derivative-quenching red-fluorescent dye (BODIPY TR-X Casein, E6639, Invitrogen). Protease-catalyzed hydrolysis released highly fluorescent BODIPY TR-X dye-labeled peptides. The accompanying increase in fluorescence is proportional to MMP3 proteolytic activity and was monitored with a microplate reader. A control without BODIPY casein was used to subtract residual fluorescence background.

### Cell Scatter Assay

SCp2 cells were seeded in six-well plates at low density (1×10⁵ cells per well), allowed to form colonies (□48 h), and serum-starved for 24 h. Epithelial cell islets were then stimulated with 9 nM epidermal growth factor (EGF) (Sigma) and imaged at 48 h with a Zeiss Imager Z1 microscope using a 10× objective.

### Immunofluorescence

SCp2 cells were cultured for 72 h on glass coverslips, fixed with 4% paraformaldehyde/PBS for 10 min, washed with PBS, and permeabilized in 0.25% Triton X-100/PBS for 10 min. Samples were blocked with 1% BSA and 5% goat serum/PBS for 1 h, followed by incubation with the primary antibody in blocking buffer overnight at 4° C. and the secondary antibody for 1 h at room temperature. Images were acquired with an upright Zeiss LSM710 using a 1.4 NA 63× oil immersion.

### Morphometry Analysis

Cell edges were outlined in F-actin-stained cells using an “Object Identification Module” from CellProfiler software (Carpenter et al. 2006). Cellular elliptical factors, defined as the ratio of the longest (length) to the shortest (width) axis of the cell, were calculated for 100 random cells per culture.

### Invasion Assay

Cell culture inserts (8 μm, 24-well format; BD Biosciences) were evenly coated with 20 μL of diluted (1:5 in DMEM/F-12 medium) Matrigel (BD Biosciences). Cells (1×10⁵) in 200 μL of DMEM/F-12 medium or different CM (as indicated in each experiment) were added to the upper compartment of the chamber. The lower compartment of the chamber was filled with 300 μL of medium containing 10% FBS as a chemoattractant. After 48 h of incubation at 37° C., the top side of the insert was cleared from noninvasive cells with a cotton swab and washed with serum-free DMEM/F-12. The remaining (invasive) cells at the lower surface of the filter were fixed and stained with a solution of Coomassie Blue 0.125% in methanol:acetic acid:H₂O (45%:10%:45% [v/v/v]) for 15 min. Invasive cells were scored by counting 10×20 magnification fields per filter with a Zeiss Imager Z1 microscope using a 20× objective. Mouse embryonic fibroblast NIH/3T3 cells were routinely included as a positive control. Results are expressed as mean±SD from three independent experiments.

### Western Blotting

Cells were lysed with a buffer containing 1% Triton X-100, 1% NP-40, and protease and phosphatase inhibitor cocktails (Calbiochem/EMD Millipore) in PBS, and the lysates were clarified by centrifugation at 16,000 g for 15 min. Protein concentration was determined using the BCA Protein Assay kit (Thermo Scientific) according to the manufacturer's instructions. Protein samples were mixed with electrophoresis sample buffer containing 5% (v/v) 2-β-mercaptoethanol and 5% (v/v) bromophenol blue and boiled for 5 min at 95° C. Samples were loaded in equal amounts into precast 4%-20% gradient polyacrylamide gels (Invitrogen) and separated by SDS-PAGE. Resolved proteins were transferred to a nitrocellulose membrane (Whatman) at 130 V for 90 min, followed by blocking of nonspecific binding with 5% BSA in 0.05% Tween-20/PBS for 1 h at room temperature. The membranes were probed with primary antibodies specific to each protein overnight at 4° C. and then with HRP-conjugated secondary antibodies (Thermo Scientific and Santa Cruz Biotechnology). Blots were visualized with an ECL detection system (Thermo Scientific) according to the manufacturer's instructions, and chemiluminescent signal was captured with a FluorChem IS-8900 (Alpha Innotech). Each Western blot was done at least three times, and here we show representative experiments.

### Co-IP

For co-IP of Flag-tagged MMP3 protein complexes, CM was incubated with anti-Flag M2 antibody-conjugated agarose beads (F2426, Sigma) for 16 h at 4° C. The beads were then washed three times with 0.05% Tween/PBS, and the immune complexes were directly eluted with electrophoresis sample buffer and analyzed by Western blotting. For liquid chromatography-tandem mass spectrometry (LC-MS/MS) analysis, beads were washed with 0.05% Tween/PBS, and protein complexes were eluted with a Flag peptide (F3290, Sigma) in 0.05% Tween/PBS. Samples were then precipitated with trichloroacetic acid and reconstituted with a buffer (Invitrosol, MS10007, Invitrogen) suitable for mass spectrometry analysis.

For co-IP of HSP90β protein complexes, CM was incubated with 10 μg of control rabbit IgG or anti-HSP90β antibody for 16 h at 4° C. Precipitation was performed with protein G sepharose beads (17-0618-01, GE Healthcare) for 4 h at 4° C. The beads were then washed three times with 0.05% Tween/PBS, and the immune complexes were directly eluted with electrophoresis sample buffer and analyzed by Western blotting.

### Mice

For preparation of primary mammary epithelial organoids, FVB mice were raised until 8-wk of age and then sacrificed. Experimental animal protocols were followed in accordance with guidelines set by the Lawrence Berkeley National Laboratory's Animal Welfare and Research Committee (AWRC).

### Trypsin Digestion of Samples for LC-MS/MS Analysis

100 μg of eluted proteins from control, FL and dPEX FLAG-immunoprecipitated samples were digested by trypsin (modified, sequencing grade, Promega) at a ratio of 1:30 enzyme/protein along with 2 mM CaCl₂ and for 16 h at 37° C. Following digestion, all reactions were acidified with 90% formic acid (2% final) to stop the proteolysis. Then, samples were centrifuged for 30 min at 14,000 rpm to remove insoluble material. The soluble peptide mixtures were collected for LC-MS/MS analysis.

### Multidimensional Chromatography and Tandem Mass Spectrometry (LC-MS/MS

Peptide mixtures were pressure-loaded onto a 250 □m inner diameter (i.d.) fused-silica capillary packed first with 3 cm of 5 □m strong cation exchange material (Partisphere SCX), followed by 3 cm of 10 □m C18 reverse phase (RP) particles (Aqua). Loaded and washed microcapillaries were connected via a 2 □m filtered union (UpChurch Scientific) to a 100 □m i.d. column, which had been pulled to a 5 □m i.d. tip using a P-2000 CO2 laser puller (Sutter Instruments), then packed with 13 cm of 3 □m C18 reverse phase (RP) particles (Aqua) and equilibrated in 5% acetonitrile, 0.1% formic acid (Buffer A). This split-column was then installed in-line with a NanoLC Eskigent HPLC pump. The flow rate of channel 2 was set at 300 nL/min for the organic gradient. The flow rate of channel 1 was set to 0.5 □L/min for the salt pulse. Fully automated 11-step chromatography runs were carried out. Three different elution buffers were used: 5% acetonitrile, 0.1% formic acid (Buffer A); 98% acetonitrile, 0.1% formic acid (Buffer B); and 0.5 M ammonium acetate, 5% acetonitrile, 0.1% formic acid (Buffer C). In such sequences of chromatographic events, peptides are sequentially eluted from the SCX resin to the RP resin by increasing salt steps (increase in Buffer C concentration), followed by organic gradients (increase in Buffer B concentration). The last. chromatography step consists in a high salt wash with 100% Buffer C followed by acetonitrile gradient. The application of a 2.5 kV distal voltage electrosprayed the eluting peptides directly into an LTQ-Orbitrap XL mass spectrometer equipped with a nano-LC electrospray ionization source (ThermoFinnigan). Full MS spectra were recorded on the peptides over a 400 to 2,000 m/z range by the Orbitrap, followed by five tandem mass (MS/MS) events sequentially generated by LTQ in a data-dependent manner on the first, second, third, and fourth most intense ions selected from the full MS spectrum (at 35% collision energy). Mass spectrometer scan functions and HPLC solvent gradients were controlled by the Xcalibur data system (ThermoFinnigan).

### Database Search and Interpretation of MS/MS Datasets

Tandem mass spectra were extracted from raw files, and a binary classifier—previously trained on a manually validated data set—was used to remove the low quality MS/MS spectra. The remaining spectra were searched against a mouse protein database containing 56,871 protein sequences downloaded as FASTA-formatted sequences from EBI-IPI (database version 3.75, released on Jul. 20, 2010) (Kersey et al. 2004), and 124 common contaminant proteins, for a total of 56,871 target database sequences. To calculate confidence levels and false positive rates, we used a decoy database containing the reverse sequences of 56,871 proteins appended to the target database (Elias and Gygi 2007), and the SEQUEST algorithm (Eng et al. 1994; Yates et al. 1995) to find the best matching sequences from the combined database.

SEQUEST searches were done using the Integrated Proteomics Pipeline (IP2, Integrated Proteomics Inc.) on Intel Xeon X5450 X/3.0 PROC processor clusters running under the Linux operating system. The peptide mass search tolerance was set to 50 ppm. No differential modifications were considered. No enzymatic cleavage conditions were imposed on the database search, so the search space included all candidate peptides whose theoretical mass fell within the 50 ppm mass tolerance window, despite their tryptic status.

The validity of peptide/spectrum matches was assessed in DTASelect2 (Tabb et al. 2002) using SEQUEST-defined parameters, the cross-correlation score (XCorr) and normalized difference in cross-correlation scores (DeltaCN). The search results were grouped by charge state (+1, +2, and +3) and tryptic status (fully tryptic, half-tryptic, and non-tryptic), resulting in 9 distinct sub-groups. In each one of the sub-groups, the distribution of XCorr and DeltaCN values for (a) direct and (b) decoy database hits was obtained, and the two subsets were separated by quadratic discriminant analysis. Outlier points in the two distributions (for example, matches with very low Xcorr but very high DeltaCN were discarded. Full separation of the direct and decoy subsets is not generally possible; therefore, the discriminant score was set such that a false positive rate of 1% was determined based on the number of accepted decoy database peptides. This procedure was independently performed on each data subset, resulting in a false positive rate independent of tryptic status or charge state.

In addition, a minimum sequence length of 7 amino acid residues was required, and each protein on the final list was supported by at least two independent peptide identifications unless specified. These additional requirements—especially the latter—resulted in the elimination of most decoy database and false positive hits, as these tended to be overwhelmingly present as proteins identified by single peptide matches. After this last filtering step, the false identification rate was reduced to below 1%.

### Mass Spectrometry Analysis

Mass spectrometry analysis is described above. Scaled signal intensities were log 2 transformed and analyzed by R software.

### Statistical Analysis

Statistical analyses were performed using GraphPad Prism 5.0 software. Student's t-test (unpaired with Welch's correction, two-tailed, 95% confidence interval) was used to determine statistical significance. Statistical analyses were always performed in relation to vector control cells (unless stated otherwise).

### REFERENCES FOR EXAMPLE 1

Alcaraz, J., Buscemi, L., Grabulosa, M., Trepat, X., Fabry, B., Farre, R. and Navajas, D. (2003). Microrheology of human lung epithelial cells measured by atomic force microscopy. Biophys J 84, 2071-9.
Alcaraz, J., Mori, H., Ghajar, C. M., Brownfield, D., Galgoczy, R. and Bissell, M. J. (2011). Collective epithelial cell invasion overcomes mechanical barriers of collagenous extracellular matrix by a narrow tube-like geometry and MMP14-dependent local softening. Integr Biol (Camb).
Atkinson, J. J., Holmbeck, K., Yamada, S., Birkedal-Hansen, H., Parks, W. C. and Senior, R. M. (2005). Membrane-type 1 matrix metalloproteinase is required for normal alveolar development. Dev Dyn 232, 1079-90.
Brinckerhoff, C. E. and Matrisian, L. M. (2002). Matrix metalloproteinases: a tail of a frog that became a prince. Nat Rev Mol Cell Biol 3, 207-14.
Brinkmann, V., Foroutan, H., Sachs, M., Weidner, K. M. and Birchmeier, W. (1995). Hepatocyte growth factor/scatter factor induces a variety of tissue-specific morphogenic programs in epithelial cells. J Cell Biol 131, 1573-86.
Chun, T. H., Hotary, K. B., Sabeh, F., Saltiel, A. R., Allen, E. D. and Weiss, S. J. (2006). A pericellular collagenase directs the 3-dimensional development of white adipose tissue. Cell 125, 577-91.
Chuong, C.-M. (1998). Molecular basis of epithelial appendage morphogenesis. Austin, Tex.: R. G. Landes.
Endo, K., Takino, T., Miyamori, H., Kinsen, H., Yoshizaki, T., Furukawa, M. and Sato, H. (2003). Cleavage of syndecan-1 by membrane type matrix metalloproteinase-1 stimulates cell migration. J Biol Chem 278, 40764-70.
Fata, J. E., Mori, H., Ewald, A. J., Zhang, H., Yao, E., Werb, Z. and Bissell, M. J. (2007). The MAPK(ERK-1,2) pathway integrates distinct and antagonistic signals from TGFalpha and FGF7 in morphogenesis of mouse mammary epithelium. Dev Biol 306, 193-207.
Fata, J. E., Werb, Z. and Bissell, M. J. (2004). Regulation of mammary gland branching morphogenesis by the extracellular matrix and its remodeling enzymes. Breast Cancer Res 6, 1-11.
Forster, T. (2012). Energy migration and fluorescence. J Biomed Opt 17, 011002.
Hirai, Y., Lochter, A., Galosy, S., Koshida, S., Niwa, S. and Bissell, M. J. (1998). Epimorphin functions as a key morphoregulator for mammary epithelial cells. J Cell Biol 140, 159-69.
Itoh, Y., Kajita, M., Kinoh, H., Mori, H., Okada, A. and Seiki, M. (1999). Membrane type 4 matrix metalloproteinase (MT4-MMP, MMP-17) is a glycosylphosphatidylinositol-anchored proteinase. J Biol Chem 274, 34260-6.
Itoh, Y., Takamura, A., Ito, N., Maru, Y., Sato, H., Suenaga, N., Aoki, T. and Seiki, M. (2001). Homophilic complex formation of MT1-MMP facilitates proMMP-2 activation on the cell surface and promotes tumor cell invasion. EMBO J 20, 4782-93.
Janda, E., Litos, G., Grunert, S., Downward, J. and Beug, H. (2002). Oncogenic Ras/Her-2 mediate hyperproliferation of polarized epithelial cells in 3D cultures and rapid tumor growth via the PI3K pathway. Oncogene 21, 5148-59.
Kajita, M., Itoh, Y., Chiba, T., Mori, H., Okada, A., Kinoh, H. and Seiki, M. (2001). Membrane-type 1 matrix metalloproteinase cleaves CD44 and promotes cell migration. J Cell Biol 153, 893-904.
Kheradmand, F., Rishi, K. and Werb, Z. (2002). Signaling through the EGF receptor controls lung morphogenesis in part by regulating MT1-MMP-mediated activation of gelatinase A/MMP2. J Cell Sci 115, 839-48.
Kim, H., Laing, M. and Muller, W. (2005). c-Src-null mice exhibit defects in normal mammary gland development and ERalpha signaling. Oncogene 24, 5629-36.
Knauper, V., Will, H., Lopez-Otin, C., Smith, B., Atkinson, S. J., Stanton, H., Hembry, R. M. and Murphy, G. (1996). Cellular mechanisms for human procollagenase-3 (MMP-13) activation. Evidence that MT1-MMP (MMP-14) and gelatinase a (MMP-2) are able to generate active enzyme. J Biol Chem 271, 17124-31.
Lehti, K., Lohi, J., Juntunen, M. M., Pei, D. and Keski-Oja, J. (2002). Oligomerization through hemopexin and cytoplasmic domains regulates the activity and turnover of membrane-type 1 matrix metalloproteinase. J Biol Chem 277, 8440-8.
Levental, K. R., Yu, H., Kass, L., Lakins, J. N., Egeblad, M., Erler, J. T., Fong, S. F., Csiszar, K., Giaccia, A., Weninger, W. et al. (2009). Matrix crosslinking forces tumor progression by enhancing integrin signaling. Cell 139, 891-906.
Lu, P. and Werb, Z. (2008). Patterning mechanisms of branched organs. Science 322, 1506-9.
Meyer, T. N., Schwesinger, C., Bush, K. T., Stuart, R. O., Rose, D. W., Shah, M. M., Vaughn, D. A., Steer, D. L. and Nigam, S. K. (2004). Spatiotemporal regulation of morphogenetic molecules during in vitro branching of the isolated ureteric bud: toward a model of branching through budding in the developing kidney. Dev Biol 275, 44-67.
Mori, H., Borowsky, A. D., Bhat, R., Ghajar, C. M., Seiki, M. and Bissell, M. J. (2012). Laser scanning-based tissue autofluorescence/fluorescence imaging (LS-TAFI), a new technique for analysis of microanatomy in whole-mount tissues. Am J Pathol 180, 2249-56.
Mori, H., Gjorevski, N., Inman, J. L., Bissell, M. J. and Nelson, C. M. (2009). Self-organization of engineered epithelial tubules by differential cellular motility. Proc Natl Acad Sci USA 106, 14890-5.
Mori, H., Tomari, T., Koshikawa, N., Kajita, M., Itoh, Y., Sato, H., Tojo, H., Yana, I. and Seiki, M. (2002). CD44 directs membrane-type 1 matrix metalloproteinase to lamellipodia by associating with its hemopexin-like domain. EMBO J 21, 3949-59.
Nguyen, A. W. and Daugherty, P. S. (2005). Evolutionary optimization of fluorescent proteins for intracellular FRET. Nat Biotechnol 23, 355-60.
Ohuchi, E., Imai, K., Fujii, Y., Sato, H., Seiki, M. and Okada, Y. (1997). Membrane type 1 matrix metalloproteinase digests interstitial collagens and other extracellular matrix macromolecules. J Biol Chem 272, 2446-51.
Overall, C. M. and Kleifeld, O. (2006a). Towards third generation matrix metalloproteinase inhibitors for cancer therapy. Br J Cancer 94, 941-6.
Overall, C. M. and Kleifeld, O. (2006b). Tumour microenvironment—opinion: validating matrix metalloproteinases as drug targets and anti-targets for cancer therapy. Nat Rev Cancer 6, 227-39.
Paszek, M. J., Zahir, N., Johnson, K. R., Lakins, J. N., Rozenberg, G. I., Gefen, A., Reinhart-King, C. A., Margulies, S. S., Dembo, M., Boettiger, D. et al. (2005). Tensional homeostasis and the malignant phenotype. Cancer Cell 8, 241-54.
Petersen, O. W., Ronnov-Jessen, L., Howlett, A. R. and Bissell, M. J. (1992). Interaction with basement membrane serves to rapidly distinguish growth and differentiation pattern of normal and malignant human breast epithelial cells. Proc Natl Acad Sci USA 89, 9064-8.
Provenzano, P. P., Inman, D. R., Eliceiri, K. W. and Keely, P. J. (2009). Matrix density-induced mechanoregulation of breast cell phenotype, signaling and gene expression through a FAK-ERK linkage. Oncogene 28, 4326-43.
Reichmann, E., Ball, R., Groner, B. and Friis, R. R. (1989). New mammary epithelial and fibroblastic cell clones in coculture form structures competent to differentiate functionally. J Cell Biol 108, 1127-38.
Riggins, K. S., Mernaugh, G., Su, Y., Quaranta, V., Koshikawa, N., Seiki, M., Pozzi, A. and Zent, R. (2010). MT1-MMP-mediated basement membrane remodeling modulates renal development. Exp Cell Res 316, 2993-3005.
Sato, H., Takino, T., Okada, Y., Cao, J., Shinagawa, A., Yamamoto, E. and Seiki, M. (1994). A matrix metalloproteinase expressed on the surface of invasive tumour cells. Nature 370, 61-5.
Shaner, N. C., Steinbach, P. A. and Tsien, R. Y. (2005). A guide to choosing fluorescent proteins. Nat Methods 2, 905-9.
Simian, M., Hirai, Y., Navre, M., Werb, Z., Lochter, A. and Bissell, M. J. (2001). The interplay of matrix metalloproteinases, morphogens and growth factors is necessary for branching of mammary epithelial cells. Development 128, 3117-31.
Steffgen, K., Dufraux, K. and Hathaway, H. (2002). Enhanced branching morphogenesis in mammary glands of mice lacking cell surface beta1,4-galactosyltransferase. Dev Biol 244, 114-33.
Sympson, C. J., Talhouk, R. S., Alexander, C. M., Chin, J. R., Clift, S. M., Bissell, M. J. and Werb, Z. (1994). Targeted expression of stromelysin-1 in mammary gland provides evidence for a role of proteinases in branching morphogenesis and the requirement for an intact basement membrane for tissue-specific gene expression. J Cell Biol 125, 681-93.
Szabova, L., Yamada, S. S., Birkedal-Hansen, H. and Holmbeck, K. (2005). Expression pattern of four membrane-type matrix metalloproteinases in the normal and diseased mouse mammary gland. J Cell Physiol 205, 123-32.
Taddei, I., Deugnier, M. A., Faraldo, M. M., Petit, V., Bouvard, D., Medina, D., Fassler, R., Thiery, J. P. and Glukhova, M. A. (2008). Beta1 integrin deletion from the basal compartment of the mammary epithelium affects stem cells. Nat Cell Biol 10, 716-22.
Talhouk, R. S., Chin, J. R., Unemori, E. N., Werb, Z. and Bissell, M. J. (1991). Proteinases of the mammary gland: developmental regulation in vivo and vectorial secretion in culture. Development 112, 439-49.
Wang, F., Weaver, V. M., Petersen, O. W., Larabell, C. A., Dedhar, S., Briand, P., Lupu, R. and Bissell, M. J. (1998). Reciprocal interactions between beta1-integrin and epidermal growth factor receptor in three-dimensional basement membrane breast cultures: a different perspective in epithelial biology. Proc Natl Acad Sci USA 95, 14821-6.
Williams, J. M. and Daniel, C. W. (1983). Mammary ductal elongation: differentiation of myoepithelium and basal lamina during branching morphogenesis. Dev Biol 97, 274-90.
Wiseman, B. S., Sternlicht, M. D., Lund, L. R., Alexander, C. M., Mott, J., Bissell, M. J., Soloway, P., Itohara, S. and Werb, Z. (2003). Site-specific inductive and inhibitory activities of MMP-2 and MMP-3 orchestrate mammary gland branching morphogenesis. J Cell Biol 162, 1123-33.
Yamada, K. M. and Cukierman, E. (2007). Modeling tissue morphogenesis and cancer in 3D. Cell 130, 601-10.
Yana, I., Sagara, H., Takaki, S., Takatsu, K., Nakamura, K., Nakao, K., Katsuki, M., Taniguchi, S., Aoki, T., Sato, H. et al. (2007). Crosstalk between neovessels and mural cells directs the site-specific expression of MT1-MMP to endothelial tip cells. J Cell Sci 120, 1607-14.

### REFERENCES FOR EXAMPLE 2

Carpenter A E, Jones T R, Lamprecht M R, Clarke C, Kang I H, Friman O, Guertin D A, Chang J H, Lindquist R A, Moffat J, et al. 2006. CellProfiler: Image analysis software for identifying and quantifying cell phenotypes. Genome Biol 7: R100.
Catelli M G, Ramachandran C, Gauthier Y, Legagneux V, Quelard C, Baulieu E E, Shyamala G. 1989. Developmental regulation of murine mammary-gland 90 kDa heat-shock proteins. Biochem J 258: 895-901.
Cheng C F, Fan J, Fedesco M, Guan S, Li Y, Bandyopadhyay B, Bright A M, Yerushalmi D, Liang M, Chen M, et al. 2008. Transforming growth factor a (TGFa)-stimulated secretion of HSP90a: Using the receptor LRP-1/CD91 to promote human skin cell migration against a TGFb-rich environment during wound healing. Mol Cell Biol 28: 3344-3358.
Cowen L E, Lindquist S. 2005. Hsp90 potentiates the rapid evolution of new traits: Drug resistance in diverse fungi. Science 309: 2185-2189.
de Graauw M, Tijdens I, Smeets M B, Hensbergen P J, Deelder A M, van de Water B. 2008 Annexin A2 phosphorylation mediates cell scattering and branching morphogenesis via cofilin Activation. Mol Cell Biol 28: 1029-1040.
Desprez P, Roskelley C, Campisi J, Bissell M J. 1993. Isolation of functional cell lines from a mouse mammary epithelial cell strain: The importance of basement membrane and cell-cell interaction. Mol Cell Differ 1: 99-110.
Dufour A, Sampson N S, Zucker S, Cao J. 2008. Role of the hemopexin domain of matrix metalloproteinases in cell migration. J Cell Physiol 217: 643-651.
Eustace B K, Sakurai T, Stewart J K, Yimlamai D, Unger C, Zehetmeier C, Lain B, Torella C, Henning S W, Beste G, et al. 2004. Functional proteomic screens reveal an essential extracellular role for hsp90a in cancer cell invasiveness. Nat Cell Biol 6: 507-514.
Farina A R, Tacconelli A, Cappabianca L, Gulino A, Mackay A R. 2002. Inhibition of human MDA-MB-231 breast cancer cell invasion by matrix metalloproteinase 3 involves degradation of plasminogen. Eur J Biochem 269: 4476-4483. Fata J E, Werb Z, Bissell M J. 2004. Regulation of mammary gland branching morphogenesis by the extracellular matrix and its remodeling enzymes. Breast Cancer Res 6: 1-11.
Fata J E, Mori H, Ewald A J, ZhangH, Yao E, Werb Z, Bissell M J. 2007. The MAPK(ERK-1,2) pathway integrates distinct and antagonistic signals from TGFa and FGF7 inmorphogenesis of mouse mammary epithelium. Dev Biol 306: 193-207.
Fotouhi N, Lugo A, Visnick M, Lusch L, Walsky R, Coffey J W, Hanglow A C. 1994. Potent peptide inhibitors of stromelysin based on the prodomain region of matrix metalloproteinases. J Biol Chem 269: 30227-30231.
Glasheen B M, Kabra A T, Page-McCaw A. 2009. Distinct functions for the catalytic and hemopexin domains of a Drosophila matrix metalloproteinase. Proc Natl Acad Sci 106: 2659-2664.
Hirai Y, Lochter A, Galosy S, Koshida S, Niwa S, Bissell M J. 1998. Epimorphin functions as a key morphoregulator for mammary epithelial cells. J Cell Biol 140: 159-169.
Houghton A M, Hartzell W O, Robbins C S, Gomis-Ruth F X, Shapiro S D. 2009. Macrophage elastase kills bacteria within murine macrophages. Nature 460: 637-641.
Iioka H, Ueno N, Kinoshita N. 2004. Essential role of MARCKS in cortical actin dynamics during gastrulation movements. J Cell Biol 164: 169-174.
Khokha R, Werb Z. 2011. Mammary gland reprogramming: Metalloproteinases couple form with function. Cold Spring Harb Perspect Biol 3: a0044333.
Kouros-Mehr H, Werb Z. 2006. Candidate regulators of mammary branching morphogenesis identified by genome-wide transcript analysis. Dev Dyn 235: 3404-3412.
Li W, Li Y, Guan S, Fan J, Cheng C F, Bright A M, Chinn C, Chen M, Woodley D T. 2007. Extracellular heat shock protein-90a: Linking hypoxia to skin cell motility and wound healing. EMBO J 26: 1221-1233.
Lochter A, Galosy S, Muschler J, Freedman N, Werb Z, Bissell M J. 1997a. Matrix metalloproteinase stromelysin-1 triggers a cascade of molecular alterations that leads to stable epithelial- to-mesenchymal conversion and a premalignant phenotype in mammary epithelial cells. J Cell Biol 139: 1861-1872.
Lochter A, Srebrow A, Sympson C J, Terracio N, Werb Z, Bissell M J. 1997b. Misregulation of stromelysin-1 expression in mouse mammary tumor cells accompanies acquisition of stromelysin-1-dependent invasive properties. J Biol Chem 272: 5007-5015.
Lu P, Werb Z. 2008. Patterning mechanisms of branched organs. Science 322: 1506-1509.
Maroun C R, Naujokas M A, Holgado-Madruga M, Wong A J, Park M. 2000. The tyrosine phosphatase SHP-2 is required for sustained activation of extracellular signal-regulated kinase and epithelial morphogenesis downstream from the met receptor tyrosine kinase. Mol Cell Biol 20: 8513-8525.
Mori H, Tomari T, Koshikawa N, Kajita M, Itoh Y, Sato H, Tojo H, Yana I, Seiki M. 2002. CD44 directs membrane-type 1 matrix metalloproteinase to lamellipodia by associating with its hemopexin-like domain. EMBO J 21: 3949-3959. Mori H, Lo A T, Inman J L, Alcaraz J, Ghajar C M, Mott J D, Nelson C M, Chen C S, Zhang H, Bascom J L, et al. 2013. Transmembrane/cytoplasmic, rather than catalytic, domains of Mmp14 signal to MAPK activation and mammary branching morphogenesis via binding to integrin b1. Development 140: 343-352.
Noe V, Fingleton B, Jacobs K, Crawford H C, Vermeulen S, Steelant W, Bruyneel E, Matrisian L M, Mareel M. 2001.
Release of an invasion promoter E-cadherin fragment by matrilysin and stromelysin-1. J Cell Sci 114: 111-118.
Overall C M, Kleifeld O. 2006. Tumour microenvironment—opinion: Validating matrix metalloproteinases as drug targets and anti-targets for cancer therapy. Nat Rev Cancer 6: 227-239.
Queitsch C, Sangster T A, Lindquist S. 2002. Hsp90 as a capacitor of phenotypic variation. Nature 417: 618-624.
Radisky D C, Levy D D, Littlepage L E, Liu H, Nelson C M, Fata J E, Leake D, Godden E L, Albertson D G, Nieto M A, et al. 2005. Rac1b and reactive oxygen species mediate MMP-3-induced EMT and genomic instability. Nature 436: 123-127.
Reichmann E, Ball R, Groner B, Friis R R. 1989. New mammary epithelial and fibroblastic cell clones in coculture form structures competent to differentiate functionally. J Cell Biol 108: 1127-1138
Rutherford S L, Lindquist S. 1998. Hsp90 as a capacitor for morphological evolution. Nature 396: 336-342.
Sakamoto T, Seiki M. 2009. Cytoplasmic tail of MT1-MMP regulates macrophage motility independently from its protease activity. Genes Cells 14: 617-626.
Schnelzer A, Prechtel D, Knaus U, Dehne K, Gerhard M, Graeff H, Harbeck N, Schmitt M, Lengyel E. 2000. Rac1 in human breast cancer: Overexpression, mutation analysis, and characterization of a new isoform, Rac1b. Oncogene 19: 3013-3020.
Sharp S Y, Boxall K, Rowlands M, Prodromou C, Roe S M, Maloney A, Powers M, Clarke P A, Box G, Sanderson S, et al. 2007. In vitro biological characterization of a novel, synthetic diaryl pyrazole resorcinol class of heat shock protein 90 inhibitors. Cancer Res 67: 2206-2216.
Simian M, Hirai Y, Navre M, Werb Z, Lochter A, Bissell M J. 2001. The interplay of matrix metalloproteinases, morphogens and growth factors is necessary for branching of mammary epithelial cells. Development 128: 3117-3131.
Sims J D, McCready J, Jay D G. 2011. Extracellular heat shock protein (Hsp)70 and Hsp90a assist in matrix metalloproteinase-2 activation and breast cancer cell migration and invasion. PLoS ONE 6: e18848.
Sternlicht M D, Lochter A, Sympson C J, Huey B, Rougier J P, Gray J W, Pinkel D, Bissell M J, Werb Z. 1999. The stromal proteinase MMP3/stromelysin-1 promotes mammary carcinogenesis. Cell 98: 137-146.
Sympson C J, Talhouk R S, Alexander C M, Chin J R, Clift S M, Bissell M J, Werb Z. 1994. Targeted expression of stromelysin-1 in mammary gland provides evidence for a role of proteinases in branching morphogenesis and the requirement for an intact basement membrane for tissue-specific gene expression. J Cell Biol 125: 681-693.
Taipale M, Jarosz D F, Lindquist S. 2010. HSP90 at the hub of protein homeostasis: Emerging mechanistic insights. Nat Rev Mol Cell Biol 11: 515-528.
Talhouk R S, Chin J R, Unemori E N, Werb Z, Bissell M J. 1991. Proteinases of the mammary gland: Developmental regulation in vivo and vectorial secretion in culture. Development 112: 439-449.
Talhouk R S, Bissell M J, Werb Z. 1992. Coordinated expression of extracellular matrix-degrading proteinases and their inhibitors regulates mammary epithelial function during involution. J Cell Biol 118: 1271-1282.
Tam E M, Wu Y I, Butler G S, Stack M S, Overall C M. 2002. Collagen binding properties of the membrane type-1 matrix metalloproteinase (MT1-MMP) hemopexin C domain. The ectodomain of the 44-kDa autocatalytic product of MT1-MMP inhibits cell invasion by disrupting native type I collagen cleavage. J Biol Chem 277: 39005-39014.
Thomasset N, Lochter A, Sympson C J, Lund L R, Williams D R, Behrendtsen O, Werb Z, Bissell M J. 1998. Expression of autoactivated stromelysin-1 in mammary glands of transgenic mice leads to a reactive stroma during early development. Am J Pathol 153: 457-467.
Vincent-Salomon A, Thiery J P. 2003. Host microenvironment in breast cancer development: Epithelial-mesenchymal transition in breast cancer development. Breast Cancer Res 5: 101-106.
Voss A K, Thomas T, Gruss P. 2000. Mice lacking HSP90b fail to develop a placental labyrinth. Development 127: 1-11. Wang P, Nie J, Pei D. 2004. The hemopexin domain of membrane-type matrix metalloproteinase-1 (MT1-MMP) is not required for its activation of proMMP2 on cell surface but is essential for MT1-MMP-mediated invasion in three-dimensional type I collagen. J Biol Chem 279: 51148-51155
Williams J M, Daniel C W. 1983. Mammary ductal elongation: Differentiation of myoepithelium and basal lamina during branching morphogenesis. Dev Biol 97: 274-290.
Wiseman B S, Sternlicht M D, Lund L R, Alexander C M, Mott J, Bissell M J, Soloway P, Itohara S, Werb Z. 2003. Site-specific inductive and inhibitory activities of MMP-2 and MMP-3 orchestrate mammary gland branching morphogenesis. J Cell Biol 162: 1123-1133.
Witty J P, Wright J H, Matrisian L M. 1995. Matrix metalloproteinases are expressed during ductal and alveolar mammary morphogenesis, and misregulation of stromelysin-1 in transgenic mice induces unscheduled alveolar development. Mol Biol Cell 6: 1287-1303
Elias J E, Gygi S P. 2007. Target-decoy search strategy for increased confidence in large-scale protein identifications by mass spectrometry. Nat Methods 4: 207-214.
Eng J K, McCormack A L, Yates III J R. 1994. An approach to correlate tandem mass spectral data of peptides with amino acid sequences in a protein database. J Am Soc Mass Spectrom 5: 976-989.
Kersey P J, Duarte J, Williams A, Karavidopoulou Y, Birney E, Apweiler R. 2004. The International Protein Index: an integrated database for proteomics experiments. Proteomics 4: 1985-1988.
Tabb D L, McDonald W H, Yates III J R. 2002. DTASelect and Contrast: Tools for Assembling and Comparing Protein Identification from Shotgun Proteomics. J Proteome Res 1: 21-26.
Yates J R, 3rd, Eng J K, McCormack A L, Schieltz D. 1995. Method to correlate tandem mass spectra of modified peptides to amino acid sequences in the protein database. Anal Chem 67: 1426-1436.

The above examples are provided to illustrate the invention but not to limit its scope. Other variants of the invention will be readily apparent to one of ordinary skill in the art and are encompassed by the appended claims. All references, publications, databases, and patents cited herein are hereby incorporated by reference for all purposes.

What is claimed is:

1. A method for inhibiting matrix metalloproteinase (MMP) proteins comprising contacting a cell with a compound that binds to a non-catalytic MMP domain and inhibits a biological effect.

2. The method of claim 1, wherein the matrix metalloproteinase (MMP) is MMP-3, MMP-9, or MMP-14.

3. The method of claim 2, wherein the matrix metalloproteinase (MMP) is MMP-14.

4. The method of claim 3, wherein the non-catalytic MMP domain comprises residues 318-582 or any fragments thereof.

5. The method of claim 4, wherein the non-catalytic MMP domain comprises residues 318-523 or any fragments thereof.

6. The method of claim 4, wherein the non-catalytic MMP domain comprises residues 542-562.

7. The method of claim 1, wherein the compound comprises a siRNA, antibody, antisense oligonucleotide or aptamer sequence.

8. The method of claim 3, wherein the compound comprises a siRNA that binds a non-catalytic MMP domain.

9. The method of claim 3, wherein the compound comprises an antibody that specifically binds to a non-catalytic MMP domain.

10. The method of claim 9, wherein the antibody is a monoclonal antibody.

11. The method of claim 10, wherein the antibody is humanized.

12. The method of claim 1, wherein the biological effect is metastasis.

13. The method of claim 1, wherein the biological effect is cell migration.

14. The method of claim 1, wherein the biological effect is tumorgenesis.

15. The method of claim 1, wherein the biological effect is tumor invasion.

16. The method of claim 2, wherein the matrix metalloproteinase (MMP) is MMP-3.

17. The method of claim 16, wherein the non-catalytic MMP domain comprises residues 289-477 or any fragments thereof.

18. The method of claim 17, wherein the non-catalytic MMP-3 domain comprises a hemopexin domain comprising residues 287-336 Hemopexin 1; residues 337-383 Hemopexin 2; residues 385-433 Hemopexin 3; and residues 434-477 Hemopexin 4 and/or any fragments thereof.

19. A method for inhibiting cancer progression comprising contacting a cell with a compound that binds to a non-catalytic MMP14 domain and inhibits metastasis, cell migration, tumorgenesis, or tumor invasion.

20. The method of claim 19, wherein the non-catalytic MMP14 domain is the transmembrane/cytoplasmic domain.

21. The method of claim 19, wherein the compound comprises a siRNA, antibody, antisense oligonucleotide or aptamer sequence.

22. The method of claim 21, wherein the compound comprises a siRNA that binds a MMP3 domain.

23. The method of claim 21, wherein the compound comprises an antibody that specifically binds to a MMP14 domain.

24. The method of claim 23, wherein the antibody is a monoclonal antibody.

25. The method of claim 24, wherein the antibody is humanized.


SEQUENCE LISTING (59 sequences)

SEQ ID NO: 1 - DNA, 3610 bp, Homo sapiens
gagaggctgtgggagaagggagggaccagaggagagagcgagagagggaaccagaccccagttcgccgactaagcagaagaaagatcaaaaaccggaaaagaggagaagagcaaacaggcactttgaggaacaatcccctttaactccaagccgacagcggtctaggaattcaagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgttcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccattcaaaaaaaaaaaaaaaaaa

SEQ ID NO: 2 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCTQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 3 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 4 - protein, 532 aa, Mus musculus
RTHTQRSPQSLSAAIAAMQKFYGLQVTGKADLATMMAMRRPRCGVPDKFGTEIKANVRRKRYAIQGLKWQHNEITFCIQNYTPKVGEYATFEAIRKAFRVWESATPLRFREVPYAYIREGHEKQADIMILFAEGFHGDSTPFDGEGGFLAHAYFPGPNIGGDTHFDSAEPWTVQNEDLNGNDIFLVAVHELGHALGLEHSNDPSAIMSPFYQWMDTENFVLPDDDRRGIQQLYGSKSGSPTKMPPQPRTTSRPSVPDKPKNPAYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSGRRPDEGTEEETEVIIIEVDEEGSGAVSAAAVVLPVLLLLLVLAVGLAVFFFRRHGTPKRLLYCQRSLLDKV

SEQ ID NO: 5 - DNA, 1828 bp, Homo sapiens
ctacaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttattttatctt

SEQ ID NO: 6 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 7 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 8 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 9 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 10 - DNA, 20 bp, Artificial sequence
  MMP14 amplification primer sequence
gagatcaaggccaatgttcg

SEQ ID NO: 11 - DNA, 20 bp, Artificial sequence
  MMP14 amplification primer sequence
gtccagggctcggcagaatc

SEQ ID NO: 12 - DNA, 19 bp, Artificial sequence
  MMP14 amplification primer sequence
catcttcttggtggctgtg

SEQ ID NO: 13 - DNA, 18 bp, Artificial sequence
  MMP14 amplification primer sequence
tgaccctgacttgcttcc

SEQ ID NO: 14 - DNA, 20 bp, Artificial sequence
  ITGB1 amplification primer sequence
ggagatgggaaacttggtgg

SEQ ID NO: 15 - DNA, 20 bp, Artificial sequence
  ITGB1 amplification primer sequence
cccattcaccccattcttgc

SEQ ID NO: 16 - DNA, 20 bp, Artificial sequence
  Control for total RNA, RT-PCR primer sequence for 18S rRNA
tcggaactgaggccatgatt

SEQ ID NO: 17 - DNA, 22 bp, Artificial sequence
  Control for total RNA, RT-PCR primer sequence for 18S rRNA
cctccgactttcgttcttgatt

SEQ ID NO: 18 - protein, 24 aa, Artificial sequence
  FLAG epitope sequence for detecting MMP3 protein
ASPTYRLYSASPASPASPASPLYS

SEQ ID NO: 19 - DNA, 36 bp, Artificial sequence
  Synthetic same sense primer sequence
cgttacgaattcatgaagagtcttccaatcctactg

SEQ ID NO: 20 - DNA, 49 bp, Artificial sequence
  Antisense primer sequence for FL
cgaatcggatcccttgtcatcgtcgtccttgtagtcacaattaagccag

SEQ ID NO: 21 - DNA, 49 bp, Artificial sequence
  DPEX antisense primer sequence
cctgcaggatcccttgtcatcgtcgtccttgtagtcgttggctggcgtc

SEQ ID NO: 22 - DNA, 36 bp, Artificial sequence; identical to SEQ ID NO: 19
  Sense Primer sequence to generate the two PCR-fragments used to create the EA construct,

SEQ ID NO: 23 - DNA, 29 bp, Artificial sequence
  Antisense primer sequence to generate the two PCR-fragments used to create the EA construct,
agcaacaagaaataaattggtccctgttg

SEQ ID NO: 24 - DNA, 30 bp, Artificial sequence
  Sense Primer sequence to generate the two PCR-fragments used to create the EA construct
gttgctgctcatgccattggccactccctg

SEQ ID NO: 25 - DNA, 49 bp, Artificial sequence; identical to SEQ ID NO: 20
  Antisense Primer sequence to generate the two PCR-fragments used to create the EA construct,

SEQ ID NO: 26 - DNA, 36 bp, Artificial sequence; identical to SEQ ID NO: 19
  Sense primer sequence used to link the two PCR fragments to form the EA construct encompassing the point mutation E219A

SEQ ID NO: 27 - DNA, 49 bp, Artificial sequence; identical to SEQ ID NO: 20
  Antisense primer sequence used to link the two PCR fragments to form the EA construct encompassing the point mutation E219A

SEQ ID NO: 28 - DNA, 4304 bp, Homo sapiens
cttgggcgttggaccccgcatcttattagcaaccagggagatttctccattttcctcttgtctacagtgcggctacaaatctgggatttttttattacttctttttttttcgaactacacttgggctcctttttttgtgctcgacttttccaccctttttccctccctcctgtgctgctgctttttgatctcttcgactaaaatttttttatccggagtgtatttaatcggttctgttctgtcctctccaccacccccacccccctccctccggtgtgtgtgccgctgccgctgttgccgccgccgctgctgctgctcgccccgtcgttacaccaacccgaggctctttgtttcccctcttggatctgttgagtttctttgttgaagaagccagcatgggtgcccagttctccaagaccgcagcgaagggagaagccgccgcggagaggcctggggaggcggctgtggcctcgtcgccttccaaagcgaacggacaggagaatggccacgtgaaggtaaacggcgacgcttcgcccgcggccgccgagtcgggcgccaaggaggagctgcaggccaacggcagcgccccggccgccgacaaggaggagcccgcggccgccgggagcggggcggcgtcgccctccgcggccgagaaaggtgagccggccgccgccgctgcccccgaggccggggccagcccggtagagaaggaggcccccgcggaaggcgaggctgccgagcccggctcgcccacggccgcggagggagaggccgcgtcggccgcctcctcgacttcttcgcccaaggccgaggacggggccacgccctcgcccagcaacgagaccccgaaaaaaaaaaagaagcgcttttccttcaagaagtctttcaagctgagcggcttctccttcaagaagaacaagaaggaggctggagaaggcggtgaggctgaggcgcccgctgccgaaggcggcaaggacgaggccgccgggggcgcagctgcggccgccgccgaggcgggcgcggcctccggggagcaggcagcggcgccgggcgaggaggcggcagcgggcgaggagggggcggcgggtggcgacccgcaggaggccaagccccaggaggccgctgtcgcgccagagaagccgcccgccagcgacgagaccaaggccgccgaggagcccagcaaggtggaggagaaaaaggccgaggaggccggggccagcgccgccgcctgcgaggccccctccgccgccgggcccggcgcgcccccggagcaggaggcagcccccgcggaggagcccgcggccgccgcagcctcgtcagcctgcgcagccccctcacaggaggcccagcccgagtgcagtccagaagcccccccagcggaggcggcagagtaaaagagcaagcttttgtgagataatcgaagaacttttctcccccgtttgtttgttggagtggtgccaggtactggttttggagaacttgtctacaaccagggattgattttaaagatgtctttttttattttacttttttttaagcaccaaattttgttgttttttttttttctcccctccccacagatcccatctcaaatcattctgttaaccaccattccaacaggtcgaggagagcttaaacaccttcttcctctgccttgtttctcttttattttttattttttcgcatcagtattaatgtttttgcatactttgcatctttattcaaaagtgtaaactttctttgtcaatctatggacatgcccatatatgaaggagatgggtgggtcaaaaagggatatcaaatgaagtgataggggtcacaatggggaaattgaagtggtgcataacattgccaaaatagtgtgccactagaaatggtgtaaaggctgtcttttttttttttttttaaagaaaagttattaccatgtattttgtgaggcaggtttacaacactacaagtcttgagttaagaaggaaagaggaaaaaagaaaaaacaccaatacccagatttaaaaaaaaaaaaacgatcatagtcttaggagttcatttaaaccataggaacttttcacttatctcatgttagctgtaccagtcagtgattaagtagaactacaagttgtataggctttattgtttattgctggtttatgaccttaataaagtgtaattatgtattaccagcagggtgtttttaactgtgactattgtataaaaacaaatcttgatatccagaagcacatgaagtttgcaactttccaccctgcccatttttgtaaaactgcagtcatcttggaccttttaaaacacaaattttaaactcaaccaagctgtgataagtggaatggttactgtttatactgtggtatgtttttgattacagcagataatgctttcttttccagtcgtctttgagaataaaggaaaaaaaaatcttcagatgcaatggttttgtgtagcatcttgtctatcatgttttgtaaatactggagaagctttgaccaatttgacttagagatggaatgtaactttgcttacaaaaattgctattaaactcctgcttaaggtgttctaattttctgtgagcacactaaaagcgaaaaataaatgtgaataaaatgtacaaatttgttgtgtttttttatgttctaataatactgagacttctaggtcttaggttaatttttaggaagatcttgcatgccatcaggagtaaattttattgtggttcttaatctgaagttttcaagctctgaaattcataatccgcagtgtcagattacgtagaggaagatcttacaacattccatgtcaaatctgttaccatttattggcatttagttttcatttaagaattgaacataattatttttattgtagctatatagcatgtcagattaaatcatttacaacaaaaggggtgtgaacctaagactatttaaatgtcttatgagaaaatttcataaagccattctcttgtcattcaggtccagaaacaaattttaaactgagtgagagtctatagaatccatactgcagatgggtcatgaaatgtgaccaaatgtgtttcaaaaattgatggtgtattacctgctattgtaattgcttagtgcttggctaatttccaaattattgcataatatgttctaccttaagaaaacaggtttatgtaacaaagtaatggtgttgaatggatgatgtcagttcatgggcctttagcatagttttaagcatcatttttttttttttttttgaaagtgtgttagcatcttgttactcaaaggataagacagacaataatacttcactgaatattaataatctttactagtttacctcctctgctctttgccacccgataactggatatcttttccttcaaaggaccctaaactgattgaaatttaagatatgtatcaaaaacattatttcatttaatgcacatctgttttgctgtttttgagcagtgtgcagtttagggttcatgataaatcattgaaccacatgtgtaacaactgaatgccaaatcttaaactcattagaaaaataacaaattaggttttgacacgcattcttaattggaataatggatcaaaaatagtggttcatgaccttaccaaacacccttgctactaataaaatcaaataacacttagaagggtatgtatttttagttagggtttcttgatcttggaggatgtttgaaagttaaaaattgaatttggtaaccaaaggactgatttatgggtctttcctatcttaaccaacgttttcttagttacctagatggccaagtacagtgcctggtatgtagtaagactcagtaaaaaagtggatttttaaaaataactcccaaagtgaatagtcaaaaatcctgttagcaaactgttatatattgctaagtttgttcttttaacagctggaatttattaagatgcattattttgattttattcactgcctaaaacactttgggtggtattgatggagttggtggattttcctccaagtgattaaatgaaatttgacgtatcttttcatccaaagttttgtacatcatgttttctaacggaaaaaaatgttaatatggcttttttgtattactaaaaatagctttgagattaaggaaaaataaataactcttgtacagttcagtattgtctattaaatctgtattggcagtatgtataatggcatttgctgtggttacaaaatacttcctctgggttataataatcatttgatccaattcctattgcttgtaaaataaagttttaccagttgatataatcaaaaaaaaaaaaaaa

SEQ ID NO: 29 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 30 - DNA, 1632 bp, Homo sapiens
gctcagcatttggggacgctctcagctctcggcgcacggcccaggtaagcggggcgcgccctgcccgcccgcgatgggccgccagctagcggggtgtggagacgctgggaagaaggcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 31 - protein, 357 aa, Homo sapiens
MGRQLAGCGDAGKKASFKMSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 32 - DNA, 1644 bp, Homo sapiens
gctcagcatttggggacgctctcagctctcggcgcacggcccaggttatcttgtagcatagcaacttcggatttcactctacccggagagtttcccgcttggttgaacacattggcctcaggaagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaaaaa

SEQ ID NO: 33 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 34 - DNA, 2017 bp, Homo sapiens
atgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaagcccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgttttttt

SEQ ID NO: 35 - protein, 632 aa, Homo sapiens
MTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 36 - DNA, 2703 bp, Homo sapiens
tttttcggaccatgacgtcaaggtgggctggtggcggcaggtgcggggttgacaatcatactcctttaaggcggagggatctacaggagggcggctgtactgtgcttcgccttatatagggcgacttggggcacgcagtagctctctcgagtcactccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttatacagttct

SEQ ID NO: 37 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 38 - DNA, 2543 bp, Homo sapiens
ctccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcagaaagcacaacgatgatgaacagtatgcttgggagtcttctgctggaggttccttcactgtgcgtgctgaccatggtgagcccattggcatgggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaagcccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttatac

SEQ ID NO: 39 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVIRKHNDDEQYAWESSAGGSFTVRADHGEPIGMGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 40 - DNA, 57 bp, Artificial sequence
  HSP90 shRNA sequence #1
ccggcaggaggagtatggcgaattctcgagaattcgccatactcctcctgctttttg

SEQ ID NO: 41 - DNA, 58 bp, Artificial sequence
  HSP90 shRNA sequence #2
ccggcatggaagaggtggattaaagctcgagctttaatccacctcttccatgtttttg

SEQ ID NO: 42 - DNA, 57 bp, Artificial sequence
  HSP90 shRNA sequence #3
ccgggctgaacaagacaaagcctatctcgagataggctttgtcttgttcagcttttt

SEQ ID NO: 43 - DNA, 58 bp, Artificial sequence
  ANXA2 shRNA sequence #1
ccgggtatgatgcttcggaactaaactcgagtttagttccgaagcatcatactttttg

SEQ ID NO: 44 - DNA, 58 bp, Artificial sequence
  ANXA2 shRNA sequence #2
ccgggagcatcaagaaagaggtcaactcgagttgacctctttcttgatgctctttttg

SEQ ID NO: 45 - DNA, 58 bp, Artificial sequence
  ANXA2 shRNA sequence #3
ccggcgagacaaggtcctgattagactcgagtctaatcaggaccttgtctcgtttttg

SEQ ID NO: 46 - DNA, 58 bp, Artificial sequence
  MARCKS protein shRNA sequence #1
ccggcttctccttcaagaagagcaactcgagttgctcttcttgaaggagaagtttttg

SEQ ID NO: 47 - DNA, 58 bp, Artificial sequence
  MARCKS protein shRNA sequence #2
ccgggccaagataatatgccactaactcgagttagtggcatattatcttggctttttg

SEQ ID NO: 48 - DNA, 58 bp, Artificial sequence
  MARCKS protein shRNA sequence #3
ccggctcctccacgtcgtcgcccaactcgagttgggcgacgacgtggaggagtttttg

SEQ ID NO: 49 - DNA, 58 bp, Artificial sequence
  MMP3 shRNA sequence #1
ccggcaagatgatgtagatggtattctcgagaataccatctacatcatcttgtttttg

SEQ ID NO: 50 - DNA, 58 bp, Artificial sequence
  MMP3 shRNA sequence #2
ccggcccacatattgaagagcaatactcgagtattgctcttcaatatgtgggtttttg

SEQ ID NO: 51 - DNA, 57 bp, Artificial sequence
  Nontargeting shRNA sequence
ccggcaacaagatgaagagcaccaactcgagttggtgctcttcatcttgttgttttt

SEQ ID NO: 52 - DNA, 58 bp, Artificial sequence
  Synthetic MMP14 hairpin sequence #1
ccggccatcaatactgcctacgaaactcgagtttcgtaggcagtattgatggtttttg

SEQ ID NO: 53 - DNA, 58 bp, Artificial sequence
  Synthetic MMP14 hairpin sequence #2
ccgggcagtgatgaagtcttcacatctcgagatgtgaagacttcatcactgctttttg

SEQ ID NO: 54 - DNA, 58 bp, Artificial sequence
  Synthetic MMP14 hairpin sequence #3
ccggccagcaactttatggaagcaactcgagttgcttccataaagttgctggtttttg

SEQ ID NO: 55 - DNA, 58 bp, Artificial sequence
  Synthetic MMP14 hairpin sequence #4
ccggcggataagtttgggactgagactcgagtctcagtcccaaacttatccgtttttg

SEQ ID NO: 56 - DNA, 58 bp, Artificial sequence
  Synthetic ITGB1 hairpin sequence #1
ccggccaagtttcaagggccaacttctcgagaagttggcccttgaaacttggtttttg

SEQ ID NO: 57 - DNA, 58 bp, Artificial sequence
  Synthetic ITGB1 hairpin sequence #2
ccggcccgacatcatcccaattgtactcgagtacaattgggatgatgtcgggtttttg

SEQ ID NO: 58 - DNA, 58 bp, Artificial sequence
  Synthetic ITGB1 hairpin sequence #3
ccgggccattactatgattatccttctcgagaaggataatcatagtaatggctttttg

SEQ ID NO: 59 - DNA, 58 bp, Artificial sequence
  Synthetic ITGB1 hairpin sequence #4
ccgggcacgatgtgatgatttagaactcgagttctaaatcatcacatcgtgctttttg